(19) United States
(12) Patent Application Publication
(10) Pub. No.: US 2010/0119461 A1
(43) Pub. Date: May 13, 2010

(54) ANTIMICROBIAL COMPOSITION

(75) Inventors: Valerie Bicard-Benhamou, Darmstadt (DE); Thomas Rudolph, Darmstadt (DE); Marcus Brunner, Stockstadt (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Merck Patent GmbH, Darmstadt (DE)

(21) Appl. No.: 12/523,938

(22) PCT Filed: Dec. 10, 2007
(86) PCT No.: PCT/EP07/10733

§ 371 Date: Jul. 21, 2009

(30) Foreign Application Priority Data
Jan. 23, 2007 (EP) 07001366.9

(51) Int. Cl. A61K 8/27; A01N 59/16; A61Q 17/04; A61Q 11/00; A61Q 5/06

(52) U.S. Cl. 424/49; 424/618; 424/59; 424/70.1

(57) ABSTRACT

The present invention relates to compositions comprising zinc oxide, barium sulphate and bound silver ions and their use in various applications, such as cosmetics, inks, lacquers or plastics.

The present invention relates to compositions comprising zinc oxide, barium sulphate and bound silver ions and their use in various applications, such as cosmetics, inks, lacquers or plastics.

Microbial contamination is an essential concern in our daily life, whether it concerns cosmetic products, surface areas in bathrooms, surgical instruments or wall paints. The usage of preservatives is a common method for preventing microbial contamination. However, the current trends show that organic preservatives are not well seen as such in view of regulatory affairs. Therefore, there is a real need of new harmless and compatible anti-microbial substances.

Silver is a known antimicrobial metal and in the past various proposals have been put forward for incorporation of silver in a composition for application. EP 0 190 504 discloses an antimicrobial composition which includes silver as the antimicrobial agent and a hydratable or hydrated oxide as a promoter to enhance the antimicrobial effect. Such compositions may be used to coat appliances such as catheters or may be incorporated in bone cements. Exemplary the hydratable or hydrated oxide is formed from element selected from silicon, titanium, aluminium or zinc.

EP 0 251 783 describes an antimicrobial composition comprising an antimicrobial silver compound, such as silver chloride or silver phosphate, deposited on a physiologically inert synthetic support material, such as oxides of titanium, aluminium or silicon in particulate form. The surface area of suitable support materials should be extended. The resulting antimicrobial composition can be dispersed in a polymeric material to prevent an antimicrobial contamination.

The combination of antibacterial activity and electrical conductivity is described in EP 0 427 858. An inorganic fine particle such as mica, alumina or titanium oxide is coated with an antibacterial metal—such as silver, copper, zinc or lead—and/or antibacterial metal compound. The resulting particles can be introduced into synthetic polymers thus obtaining antibacterial and electrically conducting polymers.

EP 0 677 989 discloses an antimicrobial powder composition comprising inorganic particles, such as the oxides of titanium, aluminium or zinc, mica or silica, having a primary surface coating of a metal or metal compound, such as silver, copper, silver oxide, silver halides, copper oxide, zinc silicate, zinc oxide or mixtures thereof, and a secondary coating providing a protective function, such as silica and alumina. The secondary coating functions as a barrier between the antimicrobial particle and a polymer matrix in which it may be incorporated. Furthermore, the secondary coating layer is believed to influence the rate at which the antimicrobial component diffuses from a dispersed particle into the polymer.

EP 0 665 004 discloses antimicrobial cosmetic pigments comprising inorganic cosmetic pigments, an amorphous glassy coating layer of metal oxide formed over the surface of said inorganic cosmetic pigment and antimicrobial metals or antimicrobial metal ions intercalated inside the lattice of said coating layer of metal oxides. By forming an additional layer onto the cosmetic pigment, the colour of the pigment changes. This is undesirable for the manufacturer of applications or formulations because he is restricted to the colours that can be achieved with pigments having the additional layer.

In all citations described above, the antimicrobial activity is introduced into the application system via a material having only an antimicrobial effect or additional layers alter the properties of the pigments.

WO 2004/092283 discloses antimicrobial pigments wherein silver ions are bound to inorganic pigments.

It is an object of the present invention to provide alternative compositions with antimicrobial activity.

Surprisingly, it has been found that compositions according to the present invention can fulfil all the objectives cited above. Therefore, the present invention is directed to a composition comprising zinc oxide, barium sulphate and bound silver ions. In the present invention “bound silver ions” means that silver in ionic form is bound covalently or ionically, preferably ionically, and especially preferably to zinc oxide and/or barium sulphate prepared according to the given example 1 below. Another preferred form of “bound silver ions” is an additional compound as defined below coated with silver ions prepared in that the additional compound is suspended in water, silver acetate is dissolved in water and the dissolved silver acetate is transferred into the first suspension followed by heating at 20° to 45° C. and the precipitation that means the additional compound coated with silver ions, is filtered by suction and dried.

In the composition of the present invention the ratio of zinc oxide to barium sulphate to bound silver ions is in the range of 12000:2000:1 to 8:1:1, based on the weight. Preferably the ratio of zinc oxide to barium sulphate to bound silver ions is in the range of 8000:1000:1 to 12:2:1 and in particular the ratio is 2500:500:1, based on the weight on the components. In this context, the amount of bound silver ions is calculated as silver oxide.

Described compositions are for example in particular suitable to be used as radiopaque dental material with the additional benefit of being anti microbial. Said compositions can be used in the prevention and treatment of colds and respiratory upper tracts through their ability as antimicrobials to reinforce the immune system. A further application uses their anti dental plaque potential in combination with their antimicrobial functionality. In all these above-mentioned applications, the complementary effects of the single components of the composition according to the present invention are used, such as the antimicrobial activity of silver ions and the ability of zinc oxide to reinforce the immune system.

It has in particular been found that zinc oxide, barium sulphate and bound silver ions have even more a synergistic antimicrobial activity. Preferably the composition according to the present invention has also a synergistic anti irritation property, synergistic anti inflammation property, synergistic wounds healing properties, synergistic anti acne properties, synergistic anti hair loss properties, synergistic properties on the reduction of sebum excretion, synergistic UV protection properties and/or synergistic effects against skin diseases, especially atopic dermatitis, in which antimicrobial activity plays direct or indirect role. This synergistic effect is surprising since there is no known relation between the antimicrobial activity of silver ions and the substances there ions might be bound to.

In a further embodiment of the present invention, the composition further comprises additional compounds, preferably in an amount of 30 to 50 weight percent, based on the composition. As a result a preferred composition of the present invention comprises, based on the composition, 30 to 50 weight percent additional compound, 40 to 60 weight percent zinc oxide, 5 to 10 weight percent barium sulphate and 0.001 to 5 weight percent bound silver ions calculated as silver oxide. particularly preferred compositions comprises 0.005 to 0.06 weight percent bound silver ions calculated as silver oxide, especially particularly preferred 0.01 to 0.04 weight percent bound silver ions calculated as silver oxide.

These additional compounds can have any known regular or irregular shape, for example the shape of platelets, spheres or needles. Preferably the additional compounds are platelet-shaped, spherical or needle-shaped.

Preferably, the additional compounds are selected from the group of natural or synthetic mica, SiO₂, TiO₂, BiOCl, aluminium oxide, glass, micaceous iron oxide, graphite, oxidised graphite, aluminium oxide-coated graphite, basic lead carbonate, barium sulphate, chromium oxide, BN, MgO, magnesium fluoride, Si₃N₄ and/or metal. Examples for metals are aluminium, titanium, silver, copper, bronze, alloys or gold, preferably aluminium or titanium. The metals can be passivated by inorganic treatment.

In particular the additional compound is selected from the group of natural or synthetic mica, SiO₂, TiO₂, aluminium oxide, glass or micaceous iron oxide, most preferably the additional compound is mica. In the latter case, the composition according to the present invention preferably comprises 40 weight percent mica, 50 weight percent zinc oxide, 9.98 weight percent barium sulphate and 0.02 weight percent bound silver ions calculated as silver oxide. The zinc oxide and the barium sulphate are preferably deposited on the surface of the additional compound, in particular on the surface of mica.

In addition, the above-mentioned additional compounds can be coated with one or more layers or deposits of BiOCl and/or transparent, semitransparent or opaque, selectively or nonselectively absorbing or nonabsorbing metal oxides, metal suboxides, metal oxide hydrates, metals, metal sulphates, metal nitrides, metal oxynitrides, metal fluorides and/or mixtures of these materials.

For the one or more layers or deposits of transparent, semitransparent or opaque, selectively or nonselectively absorbing or nonabsorbing metal oxides, metal suboxides, metal oxide hydrates, metals, metal sulphates, metal nitrides, metal oxynitrides, metal fluorides and/or mixtures of these materials all known materials can be selected. The one or more layers or deposits of transparent, semitransparent or opaque, selectively or nonselectively absorbing or nonabsorbing metal oxides, metal suboxides, metal oxide hydrates, metals, metal nitrides, metal oxynitrides, metal fluorides and/or mixtures of these materials can have a high refractive index (n>1.8) or a low refractive index (n≦1.8). The metal oxides or metal oxide hydrates can be selected from any known metal oxide or metal oxide hydrate, such as for example SiO₂, Al₂O₃, TiO₂, ZnO, ZrO₂, Ce₂O₃, FeO, Fe₂O₃, Cr₂O₃, SnO₂, silicon oxide hydrate, aluminium oxide hydrate, titanium oxide hydrate and/or mixtures thereof, such as for example ilmenite or pseudobrookite. The metal can be selected from any known metal, such as for example chromium, molybdenum, aluminium, silver, platinum, nickel, copper, gold and/or alloys, preferably from aluminium and/or silver. An example for a metal fluoride is magnesium fluoride. An example for a metal sulphate is barium sulphate. As metal nitrides or metal oxynitrides for example the nitrides or oxynitrides of titanium, zirconium and/or tantalum can be used. Preferably the one or more layer or deposit consist of metal oxides, metal oxide hydrates, metals, metal sulphates and/or metal fluorides, in particular metal oxides, metal oxide hydrates and metal sulphates. Furthermore, the additional compounds can have multilayer compositions comprising materials with a high and a low refractive index. Compositions comprising multilayer effect compounds are characterised through an intensively lustrous appearance and an angle-dependent interference colour. Preferably the one or more layers of BiOCl and/or transparent, semitransparent or opaque, selectively or nonselectively absorbing or nonabsorbing metal oxides, metal suboxides, metal oxide hydrates, metals, metal nitrides, metal oxynitrides, metal fluorides and/or mixtures of these materials are arranged as alternating layers of transparent, semitransparent or opaque, selectively or nonselectively absorbing or nonabsorbing metal oxides, metal suboxides, metal oxide hydrates, metals, metal nitrides, metal oxynitrides, metal fluorides and/or mixtures of these materials or BiOCl with a refractive index n>1.8 and transparent, semitransparent or opaque, selectively or nonselectively absorbing or nonabsorbing metal oxides, metal suboxides, metal oxide hydrates, metals, metal nitrides, metal oxynitrides, metal fluorides and/or mixtures of these materials with a refractive index n≦1.8, in particular as stack of two layers comprising one layer of a material with a high refractive index and one layer of a material with a low refractive index, whereas one or more of these stacks can be applied to the additional compound. The sequence of the layers of the material with a high refractive and the material with the low refractive index can be adapted to the material of the additional compound thus incorporating the additional compound into the multilayer composition.

Preferred examples for materials with a refractive index n>1.8 are titanium oxide, iron oxide, iron titanate, iron, chromium, silver and/or nickel, preferably titanium oxide, iron oxide, iron titanate. Preferred examples for materials with a refractive index n≦1.8 are silicon oxide, silicon oxide hydrate, aluminium oxide, aluminium oxide hydrate, aluminium and/or magnesium fluoride. In another embodiment the transparent, semitransparent or opaque, selectively or nonselectively absorbing or nonabsorbing metal oxides, metal suboxides, metal oxide hydrates, metals, metal nitrides, metal oxynitrides, metal fluorides and/or mixtures of these materials additionally may contain organic and/or inorganic colorants or elements as dopant. The absorption colour of the organic or inorganic colorant is combined with interference effects of the one or more layers of metal oxides, metal suboxides, metal oxide hydrates, metals, metal nitrides, metal oxynitrides, metal fluorides and/or mixtures of these materials thus producing pigments with special colour effects. Examples of organic colorants are azopigments, anthrachinonepigments, indigo- or thioindigo derivatives, diketo-pyrrolo-pyrrol pigments, perylen pigments or phthalocyanin pigments. Carbon black, Prussian blue, Turnbulls blue, Rinnmanns green, Thenards Blue and coloured metal oxide are only few examples of inorganic colorants, which can be introduced into the one or more layers. Yttrium or antimony can be used as dopant. Usage of these compositions can result in the reduction of the content of preservatives added to formulations and applications, thus enabling the reduction of production costs and efforts necessary by the applicant to prevent the formulations and applications to be contaminated with microorganisms.

Examples and embodiments of the above-mentioned materials and additional compounds are for example described in Research Disclosure RD 471001 and RD 472005, whose specifications are herein incorporated by reference.

The mean diameter of platelet-shaped additional compounds can vary between 1 and 200 μm, preferably 10 and 150 μm. Depending on the desired application, the size of the additional compound can accordingly be optimised. The overall thickness of the additional compound is in the range between 0.05 and 6 μm, in particular between 0.1 and 4.5 μm.

The thickness of the one or more layers or deposits of transparent, semitransparent or opaque, selectively or nonselectively absorbing or nonabsorbing metal oxides, metal suboxides, metal oxide hydrates, metals, metal sulphates, metal nitrides, metal oxynitrides, metal fluorides and/or mixtures of these materials can vary between 3 and 300 nm, preferably between 20 and 200 nm. The thickness of the metal layers or deposits is preferably in the range of 4 to 50 nm. The thickness of the deposit comprising of metal oxide and metal sulphate is preferably in the range of 4 to 50 nm. By adjusting the layer thickness the intensity of the absorption colour or the interference colours and angles can be tuned.

Depending on the material of the additional compound and the thereon-coated layers or deposits, compositions with variable colour, hiding strength, lustre and angle-dependent colour impressions are obtainable.

The preparation of above described layers or deposits can result from wet chemical treatment, from sol gel processes or by chemical or physical vapour deposition (CVD/PVD). After deposition, the resulting pigments can be dried or calcined.

Examples of additional compounds described here comprise pigments like Iriodin®, Candurin®, Timiron®, Colorstream® and Xirallic® pigments from Merck KGaA, Mearlin® and Dynacolor® pigments from Engelhard Corp., Variochrom® and Paliochrom® pigments from BASF or Spectraflair® pigments from Flex Products.

In another preferred embodiment of the present invention the additional compounds comprise spherical particles of metal oxides, for example SiO₂, TiO₂, aluminium oxide, glass, MgO, iron oxide but also BiOCl, magnesium carbonate, graphite, oxidised graphite, aluminium oxide-coated graphite, basic lead carbonate, barium sulphate, chromium oxide, BN, magnesium fluoride, Si₃N₄ and/or metals. Preferably the spherical particles comprise SiO₂, TiO₂, Al₂O₃, ZnO, Fe₂O₃, FeO and/or mixtures thereof. Furthermore, the spherical particles can be coated with one or more layers or deposits of transparent, semitransparent or opaque, selectively or nonselectively absorbing or nonabsorbing metal oxides, metal suboxides, metal oxide hydrates, metals, metal sulphates, metal nitrides, metal oxynitrides, metal fluorides and/or mixtures of these materials. The materials for the one or more layers or deposits of transparent, semitransparent or opaque, selectively or nonselectively absorbing or nonabsorbing metal oxides, metal suboxides, metal oxide hydrates, metals, metal sulphates, metal nitrides, metal oxynitrides, metal fluorides and/or mixtures of these materials can be selected from the ones described for the effect pigments.

Spherical capsules of materials described above encapsulating organic and/or inorganic compounds or materials are also suited in the sense of the definition of additional compounds applied here. The encapsulated compound or material can for example be selected for example from UV-filters. Capsules, which are to be used particularly preferably, have walls that can be obtained by a process for example described in the applications WO 00/09652, WO 00/72806 and WO 00/71084. Preference is given here to capsules whose walls are made of silica gel.

In one embodiment of the present invention the spherical particles are coated with one or more layers of transparent, semitransparent or opaque, selectively or nonselectively absorbing or nonabsorbing metal oxides, metal suboxides, metal oxide hydrates, metals, metal sulphates, metal nitrides, metal oxynitrides, metal fluorides and/or mixtures of these materials. Layers of transparent, semitransparent or opaque, selectively or nonselectively absorbing or nonabsorbing metal oxides, metal suboxides, metal oxide hydrates as an outer layer, are preferred. Particles described above can be obtained commercially, e.g. as Ronaspheres® or Eusolex®UV-Pearls™ from Merck KGaA, Darmstadt. These additional compounds are advantageous in cosmetic or pharmaceutical formulations related to their spherical shape. Compositions based on these additional compounds show, depending on the material, good wrinkle hiding effects and a good skin feeling, and can be used as fillers or in the case of the capsules as well as an active ingredient with combined features such as antimicrobial activity and for example UV-filtering activity. Furthermore, compositions based on these additional compounds also reduce the gloss of the skin and give to the skin surface a smoother appearance. In addition, the skin feeling is improved, because of the glide and roll effect of the “antimicrobial spheres”. In oral care applications for example antimicrobial low abrasive spheres can advantageously be used. These particles combine the antimicrobial activity with the low abrasive properties of the spheres.

The mean diameter of the spherical particles or capsules can vary between 5 nm and 100 μm, preferably between 8 nm and 50 μm and most preferably from 8 nm to 5 μm. Spherical metal oxides, in particular metal oxides with UV-filtering activity, preferably have a mean diameter of 5 to 100 nm, especially of 8 to 50 nm and most preferably of 8 to 30 nm. A large surface area characterizes these particles, which therefore can advantageously be used as additional substrate for compositions according to the present invention. The antimicrobial activity is combined with for example the UV-filtering activity, thus providing multifunctional materials.

The zinc oxide and/or barium sulphate are preferably deposited in the form of particles onto the surface of the additional compound.

In more detail, the most preferred embodiment of the present invention comprises a flaky powder as additional compound having particle surfaces coated with particles of barium sulphate having an average diameter of 0.1 to 2.0 μm (microns), and with needle crystal particles of zinc oxide having an average major-axis diameter of 0.05 to 1.5 μm (microns), wherein the amount in parts by weight of said barium sulphate is smaller than that of said zinc oxide, relative to the amount of said flaky powder. The flaky powder which is used for the purpose of this invention may, for example, be mica, sericite, talc or kaolin having a particle diameter of 0.5 to 100 μm (microns), and is preferably mica (muscovite).

These basic materials are known, for example as Shadeleaf A® from Merck KGaA, Germany, and can be prepared by known methods, such as for example described in EP 889099 B1. In general, such materials are prepared by suspending a flaky powder as described above in water to form its suspension; dropping (a) a water-soluble barium compound, and (b) a solution containing a member of the group consisting of sulfuric acid, sodium sulphate and potassium sulphate and containing a higher chemical stoichiometric equivalent ratio of sulphate ions than barium ions in (a), into said suspension in such a way as either dropping (b) after adding an appropriate amount of (a), or dropping (a) and (b) simultaneously, whereby the particles of said flaky powder in said suspension are coated with particles of barium sulphate; dropping (c) a water-soluble zinc compound of the group consisting of zinc sulphate and zinc acetate, and (d) a basic solution into said suspension in such a way as either dropping (d) after adding an appropriate amount of (c), or dropping (c) and (d) simultaneously, whereby said particles of said flaky powder are coated with the hydroxide or carbonate of zinc; collecting said coated particles by filtration; washing them; drying them; and calcining them. More details of the preparation methods of these materials can be found on pages 2-5 of EP 889099 B1 which are herewith incorporated by reference. Onto these preferred materials silver ions are bound, resulting in compositions according to the present invention with the above-mentioned advantages. In this case the composition not only delivers antimicrobial activity, but due to the constitution of the complete composition, mica flakes coated with zinc oxide and barium sulphate, these obtained materials can be used as effective filler with UV-shielding properties.

The preferred composition according to the present invention therefore comprises, based on the composition, 30 to 50 weight percent additional compound, 40 to 60 weight percent zinc oxide, 5 to 10 weight percent barium sulphate and 0.005 to 5 weight percent bound silver ions calculated as silver oxide, preferably the composition comprises, based on the composition, 40 weight percent additional compound, 50 weight percent zinc oxide, 9.98 weight percent barium sulphate and 0.02 weight percent bound silver ions calculated as silver oxide.

Compositions according to the present invention can be obtained in a simple way. Accordingly, methods for the preparation of compositions are also part of the present invention. A preferred process for the production of the compositions according to the present invention includes the agitation of a suspension comprising zinc oxide, barium sulphate and a silver salt as antimicrobial component. In general any silver salt can be used in the method according to the present invention; preferably the silver salt is silver oxide or silver acetate.

The process is based on a process described by A. Goetz, E. C. Y. Inn in “Reversible Photolysis of Ag Sorbed on Collodial Metal Oxides” in Rev. Modem Phys. 1948, 20, 131-142.

Preferably the pigment described in EP 889099 B1 is used in this process. As result a preferred method of the present invention comprises the agitation of a suspension comprising mica flakes coated with zinc oxide and barium sulphate, as described above, and a silver salt as antimicrobial component.

The preparation can be performed in water, ethanol, methanol, 1-propanol, 2-propanol and/or mixtures thereof, preferably water is used. The preparation temperature can vary between 10 and 60° C., preferably between 20 and 45° C. and is most preferably held at 37° C.

The suspension is agitated from 4 up to 24 hours, preferably from 8 to 20 hours, and most preferably from 10 to 18 hours.

The progress of the reaction can be easily controlled. The initial dark colour of the reaction mixture, which depends on the concentration of silver oxide, turns to colourless at the end of the reaction.

The amount of the antimicrobial compound is in the range of 0.005% to 5% by weight, preferably 0.005% to 0.6% by weight, especially preferably 0.01% to 0.04% by weight, based on the composition.

The resulting composition can be separated using any method known for a person skilled in the art. Preferably the product is filtrated or filtrated with suction and washed with water. Additionally the silver treated composition can be further washed with organic solvents, such as acetone, to remove residual water. The composition according to the present invention can be dried. Preferably the compositions are dried in an oven, most preferably at a temperature below 50° C., or by using a vacuum pump or a continuous flash evaporator, most preferably by evaporation of the solvents in vacuum.

The production process described can be performed easily and adds an antimicrobial activity.

It is believed that compositions according to the present invention are formed via an ion exchange reaction between protons or ions on the surface of zinc oxide and/or barium sulphate and silver ions resulting in silver ions bonded to moieties of the compositions, for example Zn—O⁻ moieties. These oligodynamically active structures can approximately be described as silver zincate. The source of silver ions for the reaction is silver oxide, which is only slightly soluble in water. However, the few silver ions that are at any time present in solution are capable of replacing protons on the surface area of for example zinc oxide forming water as the only reaction product besides the antimicrobial compositions. During the course of investigation further analytical experiments revealed the absence of silver metal or silver oxide simply deposited on the surface encouraging silver zincate to be the most relevant structures.

Compositions according to the present invention can be used for the inhibition of the growth and/or progeny of microorganisms, for inhibition of irritation, of inflammation, of acne formation, of sebum excretion, of hair loss, for UV protection and/or for wound healing. Microorganisms in the sense of the present invention are for example bacteria (gram positive and gram-negative bacteria), yeasts, fungi and viruses. Examples of microorganisms described herein are microorganisms selected from for example Staphylococci, Micrococci, Escherichia, Pseudomonas, Bacilli, Salmonella, Shigella, Porphyromonas, Prevotella, Wolinella, Campylobacter, Propionibacterium, Streptococci, Corynebacterium, Treponema, Fusobacterium, Bifidobacterium, Lactobacillus, Actinomyces, Candida, Malazessia, Aspergillus, herpes simplex 1 and 2.

The compositions according to the present invention show a good microbicidal activity, that means the number of germs in a medium can be reproducibly decreased. In particular the number of bacteria can be decreased by at least a factor 10³ over a time period of 14 days (starting with an inokulum of 10⁵-10⁶ bacteria/g/ml). In particular, the number of yeasts and fungi can be decreased by at least a factor 10 over a time period of 14 days (starting with an inokulum of 10⁵-10⁶ fungi or yeasts/g/ml).

The antimicrobial activity of the compositions according to the present invention can be shown by tests known for a person skilled in the art, for example those based on DIN 58940 and 58944.

Therefore, in a preferred embodiment of the invention compositions according to the present invention can be used in formulations or applications, such as for example cosmetic formulations, paints, inks, food colouring, home care products, animal care products, products for personal and work hygiene, contact lenses, chromatography materials, medical equipment, protective topicals, pharmaceutical, especially dermatological formulations, lacquers, coatings and/or plastics.

In more detail formulations and applications can mean for example antimicrobial cleansers, soaps, disinfectants, anti-fouling and antimicrobial paints for inside and outside use, antimicrobial wallpapers, antimicrobial dressings and plasters, prostheses and bone cement with antimicrobial activity, dental fillings, dental prostheses, formulations against gastrointestinal infections, active coal, antimicrobial cat litter, antimicrobial diapers, tampons or sanitary towels, ambient fragrances for rooms or cars, formulations for oral or body care, absorbent pads, air conditioning (filters and ducts), air inflated construction (air halls), agricultural and mulch films, all purpose adhesives, appliances and equipment, appliance adhesives and sealants, aprons, artificial leather, artificial plants, artificial wood, and plastic lumber, astroturf, automobile parts, automotive and truck upholstery, awnings, bags, bandages, barrier fabrics, bathroom accessories, bathtubs, bedding, beverage dispensers, bibs, boats, boat covers, book covers, bottles, brush bristles, brush handles, brooms, building components (walls, wallboard, floors, concrete, siding, roofing, shingles, hardware, carpet cleaner, ceilings and commercial and industrial applications), cable sheathing, caps (hats), cardboard, carpet and carpet underlay, caster wheels, cat litter, clinical thermometers, coats, compact discs, convertible tops, cookware, coolers, cooling towers, counter and table tops, conveyor belts, countertops, credit cards, crates (food and non-food), cups, currency, curtains, cushion pads, cutting boards, decking, dishes, dish cloths, dishwasher components, diving equipment or snorkels, drainage sewer pipe, draperies, exercise equipment, equipment for slaughterhouses or creameries or diaries, equipment for gyms, saunas or massages, fan blades, fibrefill, filters, fittings, fences, floor coverings, floor and carpet baking, flooring, foam (cushion, mattress), food preparation appliances, food and beverage processing equipment, food and drink containers, storage and bags, food handling equipment, food packaging, food and meat crates, food trays and covers, food wrap, footwear (including boots, sports equipment, and tools), fruit and vegetable brushes, fruit crates, furniture, garbage bags, garbage cans, garment bags, gaskets, general purpose containers, gloves, gowns (medical and consumers), grease traps, rigid greenhouses, greenhouse films, grout and joint compound, heating, ventilation and air conditioning, hoses, ice-making equipment and trays, incontinence care products, indoor and outdoor furniture, industrial equipment, inflatable bed, insulation for wire and cable, insulators, intimate apparel, jacket liners, janitorial equipment, kitchen and bathroom hardware, kitchen sinks and fixtures, kitchen towels, laminate and tile adhesives, laying batteries, life vests, liners, mats, mattress cover pads and filing, mattress adhesives, medical and dental apparel, mobile homes, mobile toilets, mops, money, natural and synthetic fibres and fabrics, non-woven fabrics, outerwear, packaging, pallets, paper products (wipes, tissues, wall coverings, towels, book covers, mulch), pillow covers, pipes, pipe sealant and insulating materials, plaster, plastic films, plates and utensils, playground equipment, plumbing supplies and fixtures (including toilet bowl seats), plumbing adhesives and sealants, pool liners, process vessels, protective covers, refrigerator components, roofing sheets, membranes, shingles and flashing, ropes, rugs, sales counter, sails, sanitary pipes, sealing compounds for bathrooms, kitchens or glass, sheets and blankets, shoes, shoe insoles, shower curtains, shower tubs, siding for housing, silage wrap, silos, sinks, siphons, skylights, sleeping bags, sleepwear, socks and hosiery, sponges, sprinkler, sportswear and sports equipment, storage containers, stucco, sun roof, sun shades, napkins, tanks, tape, tarps, telephone boxes or public phones, tents and other outdoor equipment, ticking (mattress pillow), tiles, tile grout, toothbrush handle and bristles, toilet paper and handkerchiefs, toilet blocks and cleaners, towels, toothbrush tumbler, toys, trim for outerwear and garments, trunk liners, tubing, umbrellas, undergarments, uniforms, upholstery, vacuum cleaner bags, wall and floor covering, wallpaper, waste bags, water tanks, waste containers, water treatment, water and ice handling equipment and filters, wet suits, wipes, wire and cable, wood, wood filled plastics.

Preferably the formulation comprising compositions according to the present invention is a cosmetic formulation. Cosmetic formulations can be in the form of solutions, suspensions, emulsions, pasta, ointments, gels, creams, lotions, powders, oils, pencils, deodorant-cremes, gels, lotions, emulsions, deodorant sticks, Roll-ons, sprays and pump sprays or lacquers, especially nail lacquers. In the case of nail lacquers comprising compositions according to the present invention they can be used as well for cosmetic aspects as well as for the treatment or prevention of nail mycosis. The combination of the colour effect with the antimicrobial activity is therefore advantageous. In all these applications the antimicrobial activity of the compositions according to the present invention can advantageously be used. For example, pigment preparations or mixtures comprising compositions are stable and can be stored over a long period of time, thus facilitating the storage and consumption of these mixtures and preparations for the user. In particular in the case of water-based inks, paints and preparations, the antimicrobial activity is of great importance due to rapid fouling and contamination with bacteria of materials in these application areas. The amount of compositions in all these formulations and applications is not crucial per se and can be adapted in each case to obtain the most effective result. Depending on the formulation or application the content preferably lies in the range of 0.1% to 70% per weight, based on the formulation or application.

In all above-mentioned applications the compositions according to the present invention can advantageously be combined with all known preservatives or antimicrobial agents, such as for example phenoxyethanol, triclosan, 7-ethylbicyclooxazolidine, benzoic acid, bronopol, butylparaben, chlorphenesin, diazolidinyl urea, dichlorobenzyl alcohol, dimethyl oxazolidine, DMDM hydantoin, ethylparaben, hexamidine diisethionate, imidiazolidinyl urea, imidiazolidinyl urea NF, iodopropynyl butylcarbamate, isobutylparaben, methylparaben, potassium sorbate NF FCC, propylparaben, quaternium-15, sodium benzoate NF FCC, sodium caprylate, sodium dehydroacetate, sodium dehydroacetate FCC, sodium hydroymethylglycinate, sodium hydroxymethylglycinate, sodium methylparaben, sodium propylparaben, sorbic acid NF FCC, anisic acid, benzethonium chloride, caprylic/capric glycerides, caprylyl glycol, di-alpha-tocopherol, ethylhexylglycerin, glyceryl caprate, methyl isothiazolinone, polymethoxy bicyclic oxazolidine. Tocopheryl acetate, alcohol, benzalkonium chloride, benzethonium chloride, camellia sinensis leaf extract, candida bombicola/glucose/methyl rapeseedate, hydrogen peroxide, methylbenzethonium chloride phenol, pinus pinaster bark extract, Poloxamer 188, PVP-Iodine, Rosmarinus officinalis Leaf extract, Vitis vinifera seed extract, ammomium benzoate, ammonium propioante, 5-Bromo-5-nitro-1,3-dioxane, Chloroxylenol, Ethyl alcohol, Glutaral, Iodopropynyl butylcarabamate, Isothiazolinone, Parabens, Pircotone olamine, Selenium disulphine, Sorbic acid (mold), Zinc pyrithione, Benzalkonium chloride, Benzethonium chloride, Benzoic acid, Dehydroacetic acid, Dimethyl hydroxmethyl pyrazole, Formaldehyde, Hexetidine, Mthyldibromo glutaronitrile, Salicylic acid, Sodium hydroxymethylglycinate, Sodium iodate, Zinc oxide, Benzyl alcohol (mould), Boric acid (yeast), Chloroacetamide, Phenoxythanol, Ortholphenylphenol, Benzalkonium chloride, Benzethonium chloride, 5-Bromo-5-nitro-1,3-dioxane, Bronopol, Diazolidinyl urea, Dimethyl hydroxmethyl pyrazole, Dimethyl oxazolidine, DMDM hydantoin, Ethyl alcohol, 7-Ethyl bicycloxazolidine, Formaldehyde, Glutaral, Imidazolidinyl urea, Isothiazolinone, Methenammonium chloride, Methylbromo glutaronitrile, Parabens, Polymethoxy bicylooxazolidine, Quaternium-15, Sodium hydroxymethylglycinate, Thimersal, Benzoic acid, Benzyl alcohol, Chlorhexidine, Hexetidine, Phenethyl alcohol, Polyaminopropyl biguanide, Polyquarternium-42, Salicylic acid, Sodium iodate, Triclocarban, Triclosan, Zinc phenolsulphonate, Chloroacetamide, Chlorobutanol, Dehydroacetic acid, Neem seed oil, Parabens, Phenoxyethanol, Tee trea oil, Usnic acid, Ammonim Benzoate, Ammonium Propionate, Benziosthiazolinone, Benzoic Acid, Benzotriazole, Benzyl Alcohol, Benzylhemiformal, Benylparaben, 5-Bromo-5-Nitro-1,3-Dioxane, 2-Bromo-2-Notropropane-1,3-Diol, Butyl Benzoate, Butylparaben, Calcium Benzoate, Calcium Paraben, Calcium Propionate, Calcium Salicylate, Calcium Sorbate, Captan, Chloramine T, Chlorhexidine Diacetate, Chlorhexidine Digluconate, Chlorhexidine Dithydrochloride, Chloroacetamine, Chlorobutanol, p-Chloro-m-Cresol, Chlorophene, p-Chlorophenol, Chlorothymol, Chloroxylenol, Citrus Grandis (Grapefruit) Fruit Extract, Citrus Grandis (Grapefruit) Seed Extract, Copper Usnate, m-Cresol, o-Cresol, p-Cresol, DEDM Hydantoin, DEDM Hydantoin Dilaurate, Dehydroacetic Acid, Diazolidinyl Urea, Dibromopropamidine Diisethionate, Dimethyl Hydroxymethyl Pyrazole, Dimethylol Ethylene Thiourea, Dimethyl Oxazolidine, Dithiomethylbenzamide, DMDM Hydantoin, DMHF, Domiphen Bromide, Ethyl Ferulate, Ethylparaben, Ferulic Acid, Formaldehyde, Glutaral, Glycerol Formal, Glyoxal, Hexamidine, Hexamidine Diparaben, Hexamidine Paraben, 4-Hydroxybenzoic Acid, Hydroxymethyl Dioxazabicyclooctane, Imidazolidinyl Urea, Iodopropynyl Butylcarbamate, Isobutylparaben, Isodecylparaben, Isopropyl Cresols, Isopropylparaben, Isopropyl Sorbate, Magnesium Benzoate, Magnesium Propionate, Magnesium Salicylate, MDM Hydantoin, MEA-Benzoate, MEA o-Phenylphenate, MEA-Salicylate, Methylchloroisthiazolinone, Methyldibromo Glutaronitrile, Methylisothazolinone, Methylparaben, Mixed Cresols, Nisin, PEG-5 DEDM Hydantoin, PEG-15 DEDM Hydantoin, PEG-5 Hydantoin Oleate, PEG-15 DEDM Hydantoin Stearate, Phenethyl Alcohol, Phenol, Phenoxyethanol, Phenoxyethylparaben, Phenoxyisopropanol, Phenyl Benzoate, Phenyl Mercuric Acetate, Phenyl Mercuric Benzoate, Phenyl Mercuric Borate, Phenyl Mercuric Bromide, Phenyl Mercuric Chloride, Phenylparaben, o-Phenylphenol, Polyaminopropyl Biguanide, Polyaminopropyl Biguanide Stearate, Polymethoxy Bicyclic Oxazolidine, Polyquaternium-42; Potassium Benzoate, Potassium Ethylparaben, Potassium Methylparaben, Potassium Paraben, Potassium Phenoxide, Potassium o-Phenylphenate, Potassium Propionate, Potassium Propylparaben, Potassium Salicylate, Potassium Sorbate, Propionic Acid, Propyl Benzoate, Propylparaben, Quaternium-8, Quaternium-14, Quaternium-15, Silver Borosilicate, Silver Magnesium Aluminium Phosphate, Sodium Benzoate, Sodium Butylparaben, Sodium p-Chloro-m-Cresol, Sodium Dehydroacetate, Sodium Ethylparaben, Sodium Formate, Sodium Hydroxymethane Sulfonate, Sodium Hydroxymethylglycinate, Sodium lsobutylparaben, Sodium Methylparaben, Sodium Paraben, Sodium Phenolsulfonate, Sodium Phenoxide, Sodium o-Phenylphenate, Sodium Propionate, Sodium Propylparaben, Sodium Pyrithione, Sodium Salicylate, Sodium Sorbate, Sorbic Acid, TEA-Sorbate, Thimerosal, Triclocarban, Triclosan, Undecylenoyl PEG-5 Paraben, Zinc Pyrithione or combinations thereof, such as for example Benzyl alcohol/mehtylchloroisothiazolinone/methylisothiazolinone, Benzyl alcohol/PPG-2 methyl ether/bronopol/deceth-8/iodopropynyl/butylcarbamate, Chloroacetamide sodium benzoate, Dehydroacetic acid/benzyl alcohol, Diazolidinyl urea/iodopropynyl butylcarbamate, Diazolidinyl urea/methylparaben/ethylparaben/butylparaben/propylparaben/isobutylpara ben/2-phenoxyethanol, DMDM hydantoin/iodopropynyl butylcarbamate, Glycerin/water/ethoxdiglycol/caprylyl glycol/sodium polyacrylate, Glyceryl laurate/caprylyl/phenylpropanol/dipropylene glycol, Isopropylparaben/isobutylparaben/butylparaben, Methyl chloroisothiazolinone/methyl isothiazolinone, Methyldibromo glutaronitrile/methylchloroisothiazolinone/methylisothiazolinone/phenoxyeth anol, Methyldibromo glutaronitrile/phenoxyethanol, Methylchloroisothiazolinone/methylisothiazolinone, Methylparaben/ethylparaben/butylparaben/propylparaben/butylenes glycol, Methylparaben/ethylparaben/butylparaben/propylparaben/isobutylparaben, Methylparaben/ethylparaben/butylparaben/propylparaben/isobutylparaben/2-phenoxy-ethanol/bronopol, Methylparaben/ethylparaben/butylparaben/propylparaben/1,3-butylene glycol isomer, Methylparaben/propylparaben, Methylparaben/propylparaben/benzyl alcohol, Methylparaben/propylparaben/bronopol/phenoxyethanol, Methylparaben/propylparaben/bronopol/propylene glycol, Methylparaben/propylparaben/ethylparaben, Methylparaben/propylparaben/propylene glycol/diazolidinyl urea, Phenoxyethanol/benzoic acid/dehydroacetic acid, Phenoxyethanol/benzyl alcohol/potassium sorbate/tocopherol, Phenoxylethanol/chlorphenesin/glycerin/methylparaben/benzoic acid, Phenoxyethanol/DMDMhydantoin/lodopropynyl butyl carbamate, Phenoxyethanol/DMDM hydantoin/methylparaben/propylparaben, Phenoxyethanol/isopropylparaben/isobutylparaben/butylparaben, Phenoxyethanol/methyldibromo glutaronitrile/idopropynyl butylcarbamate, Phenoxyethanol/methylparaben/butylparaben/ethylparaben/propylparaben, Phenoxyethanol/methylparaben/butylparaben/ethylparaben/propylparaben/isobutyl-paraben, Phenoxyethanol/methylparaben/isobutylparaben/butylparaben, Phenoxythanol/triethylene glycol/dichlorobenzyl alcohol, Polyaminopropyl biguanide/parabens/phenoxyethanol, PPG-2 methyl ether/sodium benzoate/potassium sorbate/iodopropynyl butylcarbamate, Propylene glycol/benzyl alcohol/methylchloroisothiazolinone/methylisothaizolinone, Propylene glycol/diazolidinyl urea/iodopropynyl butylcarbamate, Propylene glycol/diazolidinyl urea/methylparaben/propylparaben, Propylene glycol/MDMD hydantoin/methylparaben, Propylene glycol/MDMD hydantoin/methylparaben/propylparaben, Propylene glycol/lichen extract, Propylene glycol/phenoxyethanol/chlorphenesin/methylparaben, Sodium levulinate/phenylpropanol combinations. The combination of compositions according to the present invention with preservatives or antimicrobial agents shown above helps to decrease the amount of the preservative or antimicrobial agent in formulations or applications, which is advantageous with respect to the regulatory status and the compatibility with the skin, especially in topical applications.

Furthermore, compositions in formulations according to the present invention can be advantageously combined with antibiotics. Antibiotics in this sense mean all known antibiotics, for example selected from the group of Beta-lactam, Vancomycin, Macrolides, Tetracyclines, Quinolones, Fluoroquinolones, Nitrated compounds (as for instance Nitroxoline, Tilboquinol or Nitrofurantoin), Aminoglycosides, Phenicols, Lincosamids, Synergistins, Fosfomycin, Fusidic acid, oxazolidinones, Rifamycins, Polymixynes, Gramicidins, Tyrocydine, Glycopeptides, Sulfonamides or Trimethoprims. Combinations of compositions and antibiotics are advantageous with respect to the resistance of several microorganisms against certain antibiotics. A combination of antibiotics with compositions according to the present invention helps to overcome the resistance by simply decreasing the number of microorganisms, which have not been affected by the antibiotics.

In several application areas the antimicrobial activity can be useful in several stages of the processing. For example plastics or polymers comprising compositions according to the present invention can be stored in the form of Masterbatches for a long period of time, without risking the contamination of the Masterbatch with microorganisms. The Masterbatch can be processed in the same way as all known Masterbatches. The therewith-obtained products are useful in building and construction, household, items and furnishing, electrical and electronics parts, apparel, textiles and fabrics, coatings and laminates, transportation and recreation, adhesives, sealants and grouts, food contact items and water contact items, such as for example plastic bottles, bottle caps, films, coextrusion films, exterior and interior automotive parts etc, having surfaces, which again show antimicrobial activity. In particular bottles and films comprising compositions according to the present invention are of interest with respect to the decrease of the number of microorganisms in therein-packaged products and consumer goods. Also plastics or polymers used in baths, swimming pools, kitchens, joints compounds, sealing compounds or other in general in humid surroundings can advantageously be provided with compositions according to the present invention. Suitable plastics and polymers from which the articles are fabricated include synthetic, natural and semisynthetic organic polymers. Example of polymers that can be used to practice this invention include, but are not limited to, aliphatic and aromatic polyesters, including polyethylene terephthalate, polybutylene terephthalate, polyethylene isophthalate, polyhexamethylene terephthalate, polylactic acid, polyglycolic acid, and liquid crystalline polymers for high performance resins and fibers; polyester block copolymers; aliphatic and aromatic polyamides including nylon 6, nylon 66, nylon 610, nylon 11, nylon 12, nylon 1212, poly-p-phenylene terephthalamide, poly-m-phenylene isophthalamide; copolymerised polyamides; polyolefins including polyethylene, polypropylene, and copolymers thereof; vinyl polymers; including polystyrene, polyacrylonitrile, polyvinylalcohol, polyvinyl acetate, polyvinylchloride, polyvinylidene chloride, ABS resins and acrylic resins; copolymers of ethylene and vinyl acetate; fluorocarbon polymers, including polytetrafluoroethylene. polyvinylidene fluoride and polyvinyl fluoride; polyurethanes; segmented polyurethane elastomers, spandex or elastane elastomers; polyethers, including polyacetals; polyketones, polyetherether ketone (PEEK), polyether ketoneketone (PEKK); polyether and polyester block polymers; polysulfides; polysulfones: polysiloxanes such as polydimethyl siloxane; polycarbonates; thermosetting synthetic polymers such as phenol-formaldehyde copolymer, polyurethane, polyesterurethane, polyetherurethane, polyetherurethaneurea, polyesterurethaneurea; natural polymers such as cellulosics, cotton and wool; and regenerated or semi-synthetic polymers such as rayon, cuprammonium rayon, acetate rayon, triacetate rayon, reconstituted silk and polysaccharides. This group includes reasonable copolymers, terpolymers and blends of many of the species listed. Spandex is defined herein to refer to a fiber or filament made from a long chain synthetic polymer that comprises at least 85% by weight of segmented polyurethane.

The polymer articles of this invention can be, for example, in the shape of films, fibers, powders, granules or articles made there from such as containers, pipes and monofilaments for brushes. When a high degree of antimicrobial effect is desired, the molded article preferably has a large surface area.

A polymer article of the present invention having antimicrobial properties is comprised of at least one of the aforementioned compositions and at least one organic polymer.

If the antimicrobial composition is incorporated in an amount less than about 0.1% by weight, the polymer article has insufficient antimicrobial activity for any useful applications. However, it will be appreciated by those skilled in the art that if extremely fine particles are incorporated into the polymer matrix, then less than about 0.1% may be acceptable. Above about 60% by weight there is no significant increase in the antimicrobial activity of the polymer article and the physical properties of the polymer article start to show some deterioration. This limits the usefulness of the article. Furthermore, the incorporation of high levels of the antimicrobial composition is undesirable from an economic point of view and because of undesirable effects on the properties of the composite. A preferred upper level for the antimicrobial component is about 15% weight below which level there is an optimum combination of antimicrobial activity, polymer article properties and cost efficiency.

The polymer articles according to the present invention may contain other additives as well as antimicrobial compositions. They may contain, for example, polymerization catalysts, stabilizers, delustering agents, optical whitening agents, organic or inorganic pigments, inorganic fillers, plasticizers and so on. Examples of plastics which can be used here as well as preparation and processing methods can be found in RD 472005 or R. Glausch, M. Kieser, R. Maisch, G. Pfaff, J. Weitzel, Perlglanzpigmente, Curt R. Vincentz Verlag, 1996, 83 ff.

Paints and lacquers comprising compositions according to the present invention can be waterborne or solvent-based. They can be on the basis of synthetic or chemically modified natural polymers, such as for example, acryl polymers, vinyl polymers, alkyd resins, phenol resins, urea resins, melamine resins, polyester resins, cellulose nitrate, epoxy resins polyurethane resins, bitumen, tar, shellac, natural rubber or resins, and can comprise all known additives and adjuvants, such as for example sikkatives, waxes, dispersing agents, anti-blocking agents or drying agents. Paints and lacquers provided with compositions can be used for example in the automotive area or in the industrial area, in powder coatings, architectural use, as coating of wood, steel, inner walls, floors, blankets, facades or in humid surroundings thus providing the surfaces antimicrobial activity. Furthermore the coating is stabilized against attacks of microorganisms thus enhancing the durability of the coatings.

Antimicrobial compositions according to the present invention can advantageously be applied to all kinds of printing inks, such as liquid inks, UV curable inks, paste inks and paper coatings. Known preparations for these appliation areas lack sufficient stability against antimicrobial contamination, especially in water based systems. The usage of compositions according to the present invention can help to minimize the contamination with microorganisms thus allowing to decrease the necessary content of preservatives. The therewith provided preparations are stable for a long period of time. The liquid inks can be water based, based on water/alcohol mixtures or solvent based. Suitable binders for aqueous inks are acrylates, methacrylates, polyesters and polyurethanes. Binders for solvent based inks are nitrocellulose, ethylcellulose, polyamide, PVC/PVA-copolymers, polyvinylbutyrale, clorinated rubber, rosin modified phenolic resins, maleinic resins, calcium/zinc-resinate-EHEC, acrylates and mixtures thereof. Solvents which can be used in solvent based inks are ethanol, isopropanol, n-propanol, aceton, ethylacetate, isopropylacetate, n-propylacetate, methoxypropanol, ethoxypropanol, toluene, aliphatic hydrocarbons and mixtures. UV-curable printing inks are basically composed of a binder and a liquid monomer, such as epoxy acrylates, polyurethane acrylates, polyester acrylates as reactive monomers hexanediol diacrylate, di/tripropyleneglycol diacrylate, trimethylpropane triacrylate, trimethylolpropaneethoxy triacrylate and mixtures thereof. Paste inks containing compositions according to the present invention can further contain rosin modified phenolic resins, maleinic acid modified resins, alkyd resins, linseed/soibean oil based resins, hydrocarbon based resins and mineral oils, linseed oil or soybean oil as solvents. Paper coatings containing antimicrobial compositions may further contain starch, protein/casein, polyvinyl alcohol, latexes, carboxymethyl cellulose or acrylate binders.The printing inks may further contain known fillers and rheology modifiers. More information on technology and compositions of printing inks is provided by R. L. Leach, R. J. Pierce, in The Printing Ink Manual, Fifth Edition, Blueprint, London, 1993.

Furthermore, deodorants can be pigmented with compositions according to the present invention. Different forms of deodorants are in mind: deodorant-cremes, gels, lotions, emulsions, deodorant sticks, Roll-ons, sprays and pump sprays. The compositions are combined with a suitable carrier material used in deodorants. Examples of suitable carrier materials are glyceryl stearate, aluminium chlorohydrate, propylene glycol, carbomer, glycerin, dicapryl ether, ethanol, glyceryl cocoate, cylomethicone, dimethicone, dipropylene glycol, stearyl alcohol, mineral oil, phenyltrimethicone or sodium stearate. The odour production of the skin is the result from the modifications of initially odourless secretions from the apocrine glands, such as for example lipids, proteins, ammonia, steroids and reducing sugars, by microorganisms, like for example Staphylococcus, Corynebacterium or malassezia. The antimicrobial compositions are effective against the Gram-positive cocci group, for example against the Micrococcaceae family (Staphylococcus aureus, staphylococcus epidermidis, staphylococcus hominis), against the Gram-positive rods, for example against the Coryneforms family (Brevibacterium and/or corynebacterium for example) causing malodour of the skin, which can be reduced by deodorants comprising these compositions. The deodorants may comprise various adjuvants used in this type of composition, such as scents or perfumes, preservatives, electrolytes, silicone derivatives, dyes and/or pigments which colour the composition itself, or other ingredients customarily used for deodorants. Further ingredients that can be incorporated into the formulations are described later in this application in more detail.

Compositions according to the present invention can also be used for oral care, for example for prophylaxis and/or treatment of dental plaque, caries or oral malodour. Oral malodour, caries and dental plaque are caused by microorganisms, for example by Streptococcus sobrinus, Streptococcus mutans, Streptococcus gordonii, Streptococcus salivaris, Streptococcus sanguis, Actinomyces, Lactobacilli, Fusobacterium, Veillonella, Treponema. denticola, Porphyromonas. gingivalis, Bacteroides or Peptostreptococcus.

The oral composition may be formulated for use in any form of interdental or periodontal treatment and may be in the form, for example, of a dentifrice, mouthwash, toothpowder, chewing gum, lozenge, mouth spray, floss, dental paint, or glass ionomer cement. Use of the antimicrobial material of the present invention in a glass ionomer cement has the advantage of providing X-ray opacity as well as antimicrobial action.

Such compositions may, as appropriate, contain conventional materials such as, for example, humectants, surfactants, gelling agents, abrasives or low abrasive spheres, fluoride sources, desensitizing agents, flavorings, colorings, sweeteners, preservatives, structuring agents, bactericides, anti-tartar agents and anti-plaque agents.

Suitable humectants for use in dentifrice compositions include polyhydric alcohols such as xylitol, sorbitol, glycerol, propylene glycol and poly-ethylene glycols. Mixtures of glycerol and sorbitol are particularly effective. A humectant helps to prevent dentifrice compositions from hardening on exposure to air, and may also provide a moist feel, smooth texture, flowability, and a desirable sweetness in the mouth. Suitably, such humectants may comprise from about 0%-85%, preferably from about 0%-60% by weight of the oral hygiene composition.

Suitable surfactants for use in dentifrices, mouthwashes etc. are usually water-soluble organic compounds, and may be anionic, nonionic, cationic or amphoteric species. The surfactant used should preferably be reasonably stable, able to form suds throughout a wide pH range, and able to produce a foam in use.

Anionic surfactants include the water-soluble salts of C₁₀₋₁₈ alkyl sulphates (e. g. sodium lauryl sulfates), water soluble salts of C₁₀₋₁₈ ethoxylated alkyl sulphates, water soluble salts of C₁₀₋₁₈ alkyl sarcosinates, the water-soluble salts of sulfonated monoglycerides of C₁₀₋₁₈ fattyacids (e. g. sodium coconut monoglyceride sulfonates), alkyl aryl sulfonates (e. g. sodium dodecyl benzene sulfonate)and sodium salts of the coconut fatty acid amide of N-methyltaurine.

Nonionic surfactants suitable for use in oral compositions include the products of the condensation of alkylene oxide groups with aliphatic ‘or alkylaromatic species, and may be for example, polyethylene oxide condensates of alkyl phenols, ethylene oxide/propylene oxide copolymers (available from BASF Wyandotte Chemical Corporation under the trade name ‘Pluronic’), ethylene oxide/ethylene diamine copolymers, ethylene oxide condensates of aliphatic alcohols, long chain tertiary amine oxides, long chain tertiary phosphine oxides, long chain dialkyl sulfoxides and mixtures thereof. Alternatives include ethoxylated sorbitan esters such as those available from ICI under the trade name “Tween”.

Cationic surfactants are generally quaternary ammonium compounds having one C₈₋₁₈ alkyl chain and include, for example, lauryl trimethyl-ammonium chloride, cetyl trimethylammonium bromide, cetyl pyridinium chloride, di-isobutylphenoxyethoxyethyldimethylbenzylammonium chloride, coconutalkyltrimethylammonium nitrite and cetyl pyridinium fluoride. Also useful are benzyl ammonium chloride, benzyl dimethyl stearylammonium chloride, and tertiary amines having one C₁₋₁₈ hydrocarbon group and two (poly)oxyethylene groups.

Amphoteric surfactants are generally aliphatic secondary and tertiary amines comprising aliphatic species that may be branched or unbranched, and in which one of the aliphatic species is a C₈₋₁₈ species and the other contains an anionic hydrophilic group, for example, sulfonate, carboxylate, sulfate, phosphonate or phosphate. Examples of quaternary ammonium compounds are the quaternized imidazole derivatives available under the trade name ‘Miranol’ from the Miranol Chemical Company.

Suitably, the surfactant is included in an amount of from 0%-20%, preferably 0%-10% by weight of the oral hygiene composition.

Structuring agents may be required in, for example, dentifrices and gums to provide desirable textural properties and “mouthfeel”. Suitable agents include natural gum binders such as gum tragacanth, xanthan gum, gum karaya and gum arabic, seaweed derivatives such as Irish moss and alginates, smectite clays such as bentonite or hectorite, carboxyvinyl polymers and watersoluble cellulose derivatives such as hydroxyethyl cellulose and sodium carboxymethyl cellulose. Improved texture may also be achieved, for example, by including colloidal magnesium aluminium silicate. Suitably, the structuring agent is included in an amount of from 0%-5%, preferably 0%-3% by weight of the oral hygiene composition.

Abrasives should preferably be capable of cleaning and/or polishing the teeth without causing harm to dental enamel or dentine. They are used most commonly in dentifrices and toothpowders, but may also be used in mouthwashes etc. Suitable abrasives include the silica abrasives, such as hydrated silicas and silica gels, particularly silica xerogels such as those available under the trade name ‘Syloid’ from W. R. Grace and Company. Also suitable are precipitated silica materials such as those available under the trade name ‘Zeodent’ from J. M. Huber Corporation, and diatomaceous earths such as those available under the trade name ‘Celite’ from Johns-Manville Corporation. Alternative abrasives include alumina, insoluble metaphosphates such as insoluble sodium metaphosphate, calcium carbonate, dicalcium phosphate (in dihydrate and anhydrous forms), calcium pyrophosphate (including β-phase calcium) polymethoxylates and particulate thermosetting polymerised resins such as, for example, melamine-ureas, melamine-formaldehydes, urea-formaldehydes, melamine-urea-formaldehydes, cross-linked epoxides, melamines, phenolics, highly purified celluloses such as those available under the trade name ‘Elcema’ from Degussa AG, and cross-linked polyesters. Suitably, abrasives are included in an amount of from 0%-80%, preferably 0%-60% by weight of the oral hygiene composition. As well as abrasives also low abrasive spheres can be added.

Fluoride sources suitable for use in all oral hygiene compositions of the present invention include sodium fluoride, zinc fluoride, potassium fluoride, aluminium fluoride, lithium fluoride, sodium monofluorophosphate, acidulated phosphate fluoride, stannous fluoride, ammonium fluoride, ammonium bifluoride and amine fluoride.

Preferably, the fluoride source is present in an amount sufficient to provide from about 50 ppm to about 4,000 ppm fluoride ions in use. Inclusion of a fluoride source is beneficial, since fluoride ions are known to become incorporated into the hydroxyapatite of tooth enamel, thereby increasing the resistance of the enamel to decay. Fluoride is also now thought to act locally on the tooth enamel, altering the remineralisation-demineralisation balance in favor of remineralisation. Inclusion of a fluoride source is also desirable when a polyphosphate anti-calculus agent is included, in order to inhibit the enzymic hydrolysis of such polyphosphates by salivary phosphatase enzymes.

Suitable desensitizing agents include, for example, formaldehyde, potassium nitrate, tripotassium citrate, potassium chloride and strontium chloride (suitably as hexahydrate), strontium acetate (suitably as hemihydrate) and sodium citrate/Pluronic gel.

Flavoring agents may be added to increase palatability and may include, for example, oils of peppermint, spearmint, wintergreen, sassafras and clove. Sweetening agents may also be used, and these include D-tryptophan, saccharin, dextrose, aspartame, levulose, acesulfam, dihydrochalcones and sodium cyclamate. Typically, such flavoring agents are included in amounts of from 0%-5%, preferably from 0%-2% by weight of the oral hygiene composition. Coloring agents and pigments may be added to improve the visual appeal of the composition. Suitable colorants include dyes, such as FD & C blue No. 1, D &C yellow No. 10 and D & C yellow No. 3. A suitable and commonly used pigment is pigment grade titanium dioxide, which provides a strong white color.

Suitably, as described above, the formulations of the invention may include a further antimicrobial agent as a preservative and/or anti-plaque agent in combination with compositions according to the present invention. Suitable antimicrobial agents include zinc salts (such as zinc citrate), cetyl pyridinium chloride, the bis-biguanides (such as chlorhexidine), aliphatic amines, bromochlorophene, hexachlorophene, salicylanilides, quaternary ammonium compounds and triclosan. Enzymic systems providing a source of a natural biocide may be used as alternatives to or in combination with the biocides listed. For example, a system comprising lactoperoxidase and glucose oxidase may be used to generate antimicrobial amounts of hydrogen peroxide in the presence of glucose, water and oxygen.

The formulation may also comprise an anti-calculus agent. Suitable anti-calculus agents include zinc salts such as zinc citrate and zinc chloride and polyphosphates. Suitable pyrophosphates include the sodium and potassium pyrophosphates, preferably disodium pyrophosphate, dipotassium pyrophosphate, tetrasodium pyrophosphate and tetrapotassium pyrophosphate. A preferred source of pyrophosphate is a mixture of tetrasodium pyrophosphate and tetrapotassium pyrophosphate. Suitably, the ratio of tetrasodium pyrophosphate to tetrapotassium pyrophosphate is 0:1 to 3:1, preferably 0:1 to 1:1. Preferably, tetrapotassium pyrophosphate is the predominant species.

The formulation may also comprise alcohol. This component is particularly useful in mouthwash formulations, where it may be used to solubilise components that have low solubility in water.

Particularly suitable oral compositions are those in the form of a mouthwash or toothpaste.

Compositions according to the present invention can also be used for prophylaxis and/or treatment of dandruff. Dandruff is a scalp disorder that is characterized by the formation of white or grey scales, accompanied by mild itching. The scales present diffusely and in patches. Dandruff occurs most frequently and most severely in young males, is rare in children and the elderly, and is otherwise common throughout the world's adult population. Dandruff has traditionally been linked to seborrhoea, an inflammatory skin disorder that is known for producing greasy scales superimposed upon reddened skin areas. However, seborrhoea can occur without dandruff, and dandruff can develop in the absence of apparent seborrhoea. Current knowledge suggests that the term “dandruff” is best used to describe the symptom complex of scalp flaking and itching, rather than as a synonym for seborrhoea, which is a specific disease entity. Although dandruff is a possible symptom of seborrhoea, it also can potentially result from scalp irritation caused by excessive sun exposure, airborne environmental substances, and cosmetic hair products. Dandruff reflects a fundamental abnormality in the dead outer layer of skin (”the scalp“) that covers the hairy top of the head. The involved skin cells lack the ability to properly adhere to one another. Consequently, clumps of cells separate from the scalp surface as scales. The shedding of these scales produces flakes of dandruff. A relationship between dandruff and a class of yeast called malassezia furfur and malassezia globose has long been recognized. Bacteria and yeast are ordinary occupants of the human scalp. However, in those individuals with dandruff, yeast is present in significantly greater numbers than would normally be expected. Many doctors and researchers believe that inflammation caused by an immune response to the yeast produces the dandruff condition. In this case, a suitable formulation is in the form of a shampoo or lotion for rinsing out, the formulation in question being applied before or after shampooing, before or after colouring or bleaching or before or after permanent waving. It is also possible to choose a formulation in the form of a lotion or gel for styling or treating the hair, in the form of a lotion or gel for brushing or blow-waving, in the form of a hair lacquer, permanent waving composition, colorant or bleach for the hair. The cosmetic formulation may comprise various adjuvants used in this type of composition, such as surface-active agents, thickeners, polymers, softeners, preservatives, foam stabilizers, electrolytes, organic solvents, silicone derivatives, antigrease agents, dyes and/or pigments which colour the composition itself or the hair, or other ingredients customarily used for hair care. Further ingredients that can be incorporated into the formulations are described later in this application in more detail.

Furthermore compositions according to the present invention can also be used for prophylaxis and/or treatment of irritation, inflammation, hair loss, skin diseases, especially atopic dermatitis, acne and/or high sebum excretion, or can be used as wound healing or can also be used for UV protection (in particular UV triggered herpes).

Hair loss is the thinning of hair on the scalp. Alopecia can be temporary or permanent. Hair loss may occur after illness or after having a major surgery because of hormonal changes, after taking certain medicines after having ceratin fungal infections. There are also some indications that Malassezia species proportions may play a role in hair shedding and alopecia [American journal of clinical dermatology, (2006) Vol. 7, No. 4, pp. 263-6, Dermatology (Basel) (1998), 196(4), 474-477), WO 02/07248, Nematian et al. Am. J. Clin. Dermatol., 2006, 7(4), 263-266, Pierard-Franchimont et al., Int J. Cosmet. Sci. 2006, 28, 311-318].

Furthermore, compositions according to the present invention can also be used for prophylaxis and/or treatment of herpes, for example herpes labialis or herpes genitalis. The quiet pandemic herpes simplex virus (HSV) infection cannot be cured, that means after primary or initial infection the virus persists for life in a latent form, periodically reactivating and often resulting in significant psychosocial distress for the patient. The most relevant subtypes of the Herpesviridae with a high incidence rate are HSV-1 and HSV-2. The viruses are the cause of mucocutanoeus infections such as oral-facial infections (e.g. herpes labialis, pharyngitis herpetica or herpetic gingivostomatitis predominantly caused by HSV-1), cutanous infections (e.g. herpetic whitlow and herpes gladiatorum), herpes genitalis or perianal herpes (in the majority of the cases caused by HSV-2). Several in vitro studies have shown that especially silver ions are effective against HSV (e.g. F. Shimizu, Y. Shimizu, K. Kumagai, Antimicrob. Agents Chemother. 1976, 57-63). Therefore antimicrobial compositions according to the present invention can be used for the treatment of herpes. The treatment can preferably be achieved by topical administration of formulations comprising compositions according to the present invention. The formulations can be for example in the form of creams, solutions, ointments, gels, balms or sticks. For the treatment of infections of the lips, creams, gels, balms, ointments or sticks are especially preferred. In all these formulations the compositions according to the present invention can advantageously be combined with all known substances suitable for the treatment of herpes infections, such as for example acyclovir, valacyclovir, famciclovir, peniciclovir, idoxuridine, vidarabine, trifluridine, foscarnet, ribonucleotide reductase inhibitors, protease inhibitors, docosanol, tin bifluoride, zinc oxide or benzocaine. The amount of the compositions according to the present invention can vary between 0.5% to 20%, based on the formulation, in particular between 1% to 10%. Further ingredients that can be incorporated into the formulations are described later in this application in more detail.

The present invention is also directed to formulations or applications comprising compositions according to the present invention. Preferably the formulation or application may furthermore comprise at least one compound selected from the group consisting of suitable substrates for microorganisms, such as for example organic compounds. The suitable substrates for microorganisms are for example selected from the group consisting of alkanes, alkenes, alkines, with or without functional groups, sugars, polyols, alcohols, saturated or unsaturated carboxylic acids, proteins, amino acids, water, fatty acids, waxes, fats, mineral oils, salts, hormones, steroids, vitamins and/or derivatives or salts thereof. The combination of compositions of the present invention with these substrates allows the broadening of the application area of these substrates, for example in cosmetic formulations. The contamination of formulations containing these substrates is no longer an obstacle for their use. Generally the use of compositions according to the present invention in formulations allows the reduction of the amount or number of preservatives, which have to be added further to the formulation. In particular, there is no need for adding any further preservatives to the formulation.

Formulations or preparations containing compositions according to the present invention usually comprise several ingredients. In the following examples of commonly used ingredients, especially for cosmetic formulations, are given.

Preferred formulations or applications additionally comprise at least one UV filter resulting in antimicrobial preparations having light protection properties. The UV filter can preferably be selected from the group of dibenzoylmethane derivatives. The dibenzoylmethane derivatives used within the scope of the present invention are products which are already well known per se and are described, in particular, in the specifications FR-A-2 326 405, FR-A-2 440 933 and EP-A-0 114 607.

The dibenzoylmethane derivatives which can be used in accordance with the invention may be selected, in particular, from the dibenzoylmethane derivatives of the following formula:

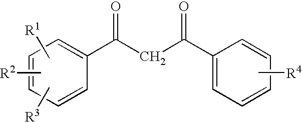

in which R¹, R², R³ and R⁴, which are identical to or different from one another, are hydrogen, a straight-chain or branched C₁₋₈-alkyl group or a straight-chain or branched C₁₋₈-alkoxy group. In accordance with the present invention, it is of course possible to use one dibenzoylmethane derivative or a plurality of dibenzoylmethane derivatives. Of the dibenzoylmethane derivatives to which the present invention more specifically relates, mention may be made, in particular, of: 2-methyldibenzoylmethane, 4-methyldibenzoylmethane, 4-isopropyldibenzoylmethane, 4-tert-butyldibenzoylmethane, 2,4-dimethyldibenzoylmethane, 2,5-dimethyldibenzoylmethane, 4,4′-diisopropyldibenzoylmethane, 4,4′-methoxy-tert-butyldibenzoylmethane, 2-methyl-5-isopropyl-4′-methoxydibenzoylmethane, 2-methyl-5-tert-butyl-4′-methoxydibenzoylmethane, 2,4-dimethyl-4′-methoxydibenzoylmethane and 2,6-dimethyl-4-tert-butyl-4′-methoxydibenzoylmethane, this list being non-restrictive.

Of the above-mentioned dibenzoylmethane derivatives, particular preference is given in accordance with the invention to 4,4′-methoxy-tert-butyldibenzoylmethane and especially 4,4′-methoxy-tert-butyldibenzoylmethane, which is commercially available under the trade name Eusolex® 9020 from Merck KGaA, where this filter conforms to the following structural formula:

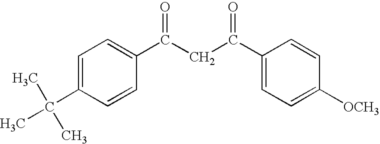

A further dibenzoylmethane derivative which is preferred in accordance with the invention is 4-isopropyldibenzoylmethane.

Additionally, in likewise preferred embodiments of the invention, the preparations according to the invention may also contain compounds of the formula I which have a UV absorption in the UV-A and/or UV-B region:

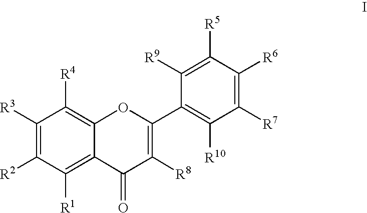

where R¹ to R¹⁰ may be identical or different and are selected from
   H
   OR¹¹
   straight-chain or branched C₁- to C₂₀-alkyl groups,
   straight-chain or branched C₃- to C₂₀-alkenyl groups,
straight-chain or branched C₁- to C₂₀-hydroxyalkyl groups, where the hydroxyl group may be bonded to a primary or secondary carbon atom of the chain and furthermore the alkyl chain may also be interrupted by oxygen, and/or
   C₃- to C₁₀-cycloalkyl groups and/or C₃- to C₁₂-cycloalkenyl groups, where the rings may each also be bridged by —(CH₂)ₙ— groups, where n=1 to 3,
   where all OR¹¹ are, independently of one another,
      OH
      straight-chain or branched C₁- to C₂₀-alkoxy groups,
      straight-chain or branched C₃- to C₂₀-alkenyloxy groups,
      straight-chain or branched C₁- to C₂₀-hydroxyalkoxy groups, where the hydroxyl group(s) may be bonded to a primary or secondary carbon atom of the chain and furthermore the alkyl chain may also be interrupted by oxygen, and/or
      C₃- to C₁₀cycloalkoxy groups and/or C₃- to C₁₂-cycloalkenyloxy groups, where the rings may each also be bridged by —(CH₂)ₙ— groups, where n=1 to 3, and/or
      mono- and/or oligoglycosyl radicals,
with the proviso that at least 3 radicals from R¹ to R⁷ are OH and that at least 2 pairs of adjacent -OH groups are present in the molecule,
   or R², R⁵ and R⁶ are OH and the radicals R¹, R³, R⁴ and R⁷⁻¹⁰ are H.

The flavonoids of the formula I to be employed in accordance with the invention include broad-band UV filters, which can be employed alone or in combination with further UV filters. Other, likewise preferred compounds of the formula I exhibit an absorption maximum in the transition region between UV-B and UV-A radiation. As UV-A-II filters, they therefore advantageously supplement the absorption spectrum of commercially available UV-B and UV-A-I filters. They are insoluble or have low solubility in the preparation matrix. In this case, the compounds are preferably dispersed in the cosmetic preparation in finely divided form. In addition, preferred compounds of this type have advantages on incorporation into the preparations:
mono- and/or oligoglycosyl radicals improve the water solubility of the compounds to be employed in accordance with the invention;
straight-chain or branched C₁- to C₂₀-alkoxy groups, in particular long-chain alkoxy functions, such as ethylhexyloxy groups, increase the oil solubility of the compounds;
   i.e. the hydrophilicity or lipophilicity of the compounds according to the invention can be controlled via a suitable choice of substituents.

Preferred mono- or oligosaccharide radicals are hexosyl radicals, in particular ramnosyl radicals and glucosyl radicals. However, other hexosyl radicals, for example allosyl, altrosyl, galactosyl, gulosyl, idosyl, mannosyl and talosyl, may also advantageously be used. It may also be advantageous to use pentosyl radicals. The glycosyl radicals may be linked to the basic structure by means of an α- or β-glycosidic link. A preferred disaccharide is, for example, 6-O-(6-deoxy-α-L-mannopyranosyl)-β-D-glucopyranoside.

On use of the dibenzoylmethane derivatives which are particularly preferred as UV-A filters in combination with the compounds of the formula I, an additional advantage arises: the UV-sensitive dibenzoylmethane derivatives are additionally stabilised by the presence of the compounds of the formula I. The present invention therefore furthermore relates to the use of the compounds of the formula I for the stabilisation of dibenzoylmethane derivatives in preparations.

In principle, all known UV filters are suitable for combination with dibenzoylmethane derivatives and with the compounds of the formula I according to the invention, for example one or more additional hydrophilic or lipophilic sun-protection filters which are effective in the UV-A region and/or UV-B region and/or IR and/or VIS region (absorbers). These additional filters can be selected, in particular, from cinnamic acid derivatives, salicylic acid derivatives, camphor derivatives, triazine derivatives, β,β-diphenyl acrylate derivatives, p-aminobenzoic acid derivatives and polymeric filters and silicone filters, which are described in the application WO 93/04665. Further examples of organic filters are indicated in Patent Application EP-A 0 487 404. Particular preference is given to UV filters whose physiological acceptability has already been demonstrated. Both for UVA and UVB filters, there are many proven substances which are known from the specialist literature, for example benzylidenecamphor derivatives, such as 3-(4′-methylbenzylidene)-dl-camphor (for example Eusolex® 6300), 3-benzylidenecamphor (for example Mexoryl® SD), polymers of N-{(2 and 4)-1(2-oxoborn-3-ylidene)methyl]-benzyl}acrylamide (for example Mexoryl® SW), N,N,N-trimethyl-4-(2-oxoborn-3-ylidenemethyl)anilinium methylsulfate (for example Mexoryl® SK) or (2-oxoborn-3-ylidene)toluene-4-sulfonic acid (for example Mexoryl® SL), benzoyl- or dibenzoylmethanes, such as 1-(4-tert-butylphenyl)-3-(4-methoxyphenyl)propane-1,3-dione (for example Eusolex® 9020) or 4-isopropyldibenzoylmethane (for example Eusolex® 8020), benzophenones, such as 2-hydroxy-4-methoxybenzophenone (for example Eusolex® 4360) or 2-hydroxy-4-methoxybenzophenone-5-sulfonic acid and its sodium salt (for example Uvinul® MS-40),

methoxycinnamic acid esters, such as octyl methoxycinnamate (for example Eusolex® 2292), isopentyl 4-methoxycinnamate, for example as a mix-ture of the isomers (for example Neo Heliopan® E 1000), salicylate derivatives, such as 2-ethylhexyl salicylate (for example Eusolex® OS), 4-isopropylbenzyl salicylate (for example Megasol®) or 3,3,5-trimethylcyclohexyl salicylate (for example Eusolex® HMS), 4-aminobenzoic acid and derivatives, such as 4-aminobenzoic acid, 2-ethylhexyl 4-(dimethylamino)benzoate (for example Eusolex® 6007) or ethoxylated ethyl 4-aminobenzoate (for example Uvinul® P25), phenylbenzimidazolesulfonic acids, such as 2-phenylbenzimidazole-5-sulfonic acid and potassium, sodium and triethanolamine salts thereof (for example Eusolex® 232), 2,2-(1,4-phenylene)bisbenzimidazole-4,6-disulfonic acid and salts thereof (for example Neoheliopan® AP) or 2,2-(1,4-phenylene)bisbenzimidazole-6-sulfonic acid;

and further substances, such as
2-ethylhexyl 2-cyano-3,3-diphenylacrylate (for example Eusolex® OCR),
3,3′-(1,4-phenylenedimethylene)bis(7,7-dimethyl-2-oxobicyclo[2.2.1]hept-1-ylmethanesulfonic acid and salts thereof (for example Mexoryl® SX),
2,4,6-trianilino-(p-carbo-2′-ethylhexyl-1′-oxy)-1,3,5-triazine (for example Uvinul® T 150) and
hexyl 2-(4-diethylamino-2-hydroxybenzoyl)benzoate (for example Uvinul® UVA Plus, BASF).

The compounds mentioned in the list should only be regarded as examples. It is of course also possible to use other UV filters. In particular organic particular UV filters, as described in WO 99/66896, can be advantageously used in formulations comprising compositions according to the present invention.

These organic UV filters are generally incorporated into cosmetic formulations in an amount of from 0.5 to 10 per cent by weight, preferably 1%-8%.

Further suitable organic UV filters are, for example,
2-(2H-benzotriazol-2-yl)-4-methyl-6-(2-methyl-3-(1,3,3,3-tetramethyl-1-(trimethylsilyloxy)disiloxanyl)propyl)phenol (for example Silatrizole®),
2-ethylhexyl 4,4′-[(6-[4-((1,1-dimethylethyl)aminocarbonyl)phenylamino]-1,3,5-triazine-2,4-diyl)diimino]bis(benzoate) (for example Uvasorb® HEB),
α-(trimethylsilyl)-ω-[trimethylsilyl)oxy]poly[oxy(dimethyl [and about 6% of methyl[2-[p-[2,2-bis(ethoxycarbonyl]vinyl]phenoxy]-1-methyleneethyl] and approximately 1.5% of methyl[3-[p-[2,2-bis(ethoxycarbonyl)vinyl]-phenoxy]propenyl] and from 0.1% to 0.4% of (methylhydrogen]silylene]] (n≈60) (CAS No. 207 574-74-1)
2,2′-methylenebis(6-(2H-benzotriazol-2-yl)-4-(1,1,3,3-tetramethylbutyl)-phenol) (CAS No. 103 597-45-1)
2,2′-(1,4-phenylene)bis(1H-benzimidazole-4,6-disulfonic acid, mono-sodium salt) (CAS N. 180 898-37-7) and
2,4-bis{[4-(2-ethylhexyloxy)-2-hydroxy]phenyl}-6-(4-methoxyphenyl)-1,3,5-triazine (CAS No. 103 597-45-, 187 393-00-6).

Further suitable UV filters are methoxyflavones corresponding to the earlier German patent application DE 10232595.2.

Organic UV filters are generally incorporated into cosmetic formulations in an amount of from 0.5 to 20 per cent by weight, preferably 1%-15% by weight.

It may furthermore be preferred in accordance with the invention for the preparations to comprise further inorganic UV filters. Preference is given here both to those from the group consisting of titanium dioxides, such as, for example, coated titanium dioxide (for example Eusolex® T-2000 or Eusolex®T-AQUA), zinc oxides (for example Sachtotec®), iron oxides and also cerium oxides. These inorganic UV filters are generally incorporated into cosmetic preparations in an amount of from 0.5 to 20 per cent by weight, preferably 2%-10% by weight. In particular, it may be preferred here for a UV-Filter to be incorporated into one phase of emulsions and a further inorganic UV filter to be incorporated into the other phase.

Preferred compounds having UV-filtering properties are 3-(4′-methylbenzyl-idene)-dl-camphor, 1-(4-tert-butylphenyl)-3-(4-methoxyphenyl)propane-1,3-dione, 4-isopropyldibenzoylmethane, 2-hydroxy-4-methoxybenzophenone, octyl methoxycinnamate, 3,3,5-trimethylcyclohexyl salicylate, 2-ethylhexyl 4-(dimethylamino)benzoate, 2-ethylhexyl 2-cyano-3,3-diphenylacrylate, 2-phenylbenzimidazole-5-sulfonic acid and its potassium, sodium and triethanolamine salts.

Combining one or more compounds of the above-mentioned UV filters can optimise the protective action against the damaging effects of UV radiation.

Optimised compositions may comprise, for example, the combination of the organic UV filters 4′-methoxy-6-hydroxyflavone with 1-(4-tert-butylphenyl)-3-(4-methoxyphenyl)propane-1,3-dione and 3-(4′-methylbenzylidene)-dl-camphor. This combination gives rise to broad-band protection, which can be supplemented by the addition of inorganic UV filters, such as titanium dioxide microparticles.

All the said UV filters can also be employed in encapsulated form. In particular, it is advantageous to employ organic UV filters in encapsulated form. In detail, the following advantages arise:
The hydrophilicity of the capsule wall can be set independently of the solubility of the UV filter. Thus, for example, it is also possible to incorporate hydrophobic UV filters into purely aqueous preparations. In addition, the oily impression on application of the preparation comprising hydrophobic UV filters, which is frequently regarded as unpleasant, is suppressed.
Certain UV filters, in particular dibenzoylmethane derivatives, exhibit only reduced photostability in cosmetic preparations. Encapsulation of these filters or compounds which impair the photostability of these filters, such as, for example, cinnamic acid derivatives, enables the photostability of the entire preparation to be increased.
Skin penetration by organic UV filters and the associated potential for irritation on direct application to the human skin is repeatedly being discussed in the literature. The encapsulation of the corresponding substances which is proposed here suppresses this effect.
In general, encapsulation of individual UV filters or other ingredients enables preparation problems caused by the interaction of individual preparation constituents with one another, such as crystallisation processes, precipitation and agglomerate formation, to be avoided since the interaction is suppressed.

It is therefore preferred in accordance with the invention for one or more of the UV filters to be in encapsulated form. It is advantageous here for the capsules to be so small that they cannot be viewed with the naked eye. In order to achieve the above-mentioned effects, it is furthermore necessary for the capsules to be sufficiently stable and the encapsulated active ingredient (UV filter) only to be released to the environment to a small extent, or not at all.

Suitable capsules can have walls of inorganic or organic polymers. For example, U.S. Pat. No. 6,242,099 B1 describes the production of suitable capsules with walls of chitin, chitin derivatives or polyhydroxylated polyamines. Capsules which can particularly preferably be employed in accordance with the invention have walls which can be obtained by sol-gel processes, as described in the applications WO 00/09652, WO 00/72806 and WO 00/71084. Preference is again given here to capsules whose walls are built up from silica gel (silica; undefined silicon oxide hydroxide). The production of corresponding capsules is known to the person skilled in the art, for example from the cited patent applications, whose contents expressly also belong to the subject-matter of the present application.

The capsules in preparations according to the invention are preferably present in amounts which ensure that the encapsulated UV filters are present in the preparation in the above-indicated amounts.

In accordance with the invention, the above-mentioned UV filters may also be provided with a surface treatment which reinforces the hydrophilic or hydrophobic properties. Suitable for hydrophobic modification is, for example, a silicone or silane coating.

As is known, the silicones are organosilicon polymers or oligomers having a straight-chain or cyclic, branched or crosslinked structure with various molecular weights which are obtained by polymerisation and/or poly-condensation with suitably functionalised silanes and are essentially formed from recurring principal units in which the silicon atoms are linked to one another via oxygen atoms (siloxane bonding), where optionally substituted hydrocarbon groups are bonded directly to the silicon atoms via a carbon atom. The most common hydrocarbon groups are alkyl groups and in particular methyl groups, fluoroalkyl groups, aryl groups and in particular phenyl groups, as well as alkenyl groups and in particular vinyl groups. Further types of group which can be bonded to the siloxane chain either directly or via a hydrocarbon group are, in particular, hydrogen, the halogens and in particular chlorine, bromine or fluorine, the thiols, alkoxy groups, polyoxyalkylene groups (or polyethers) and in particular polyoxyethylene and/or polyoxypropylene, hydroxyl groups or hydroxyalkyl groups, optionally substituted amino groups, amide groups, acyloxy groups or acyloxyalkyl groups, hydroxyalkylamino groups or aminoalkyl groups, quaternary ammonium groups, amphoteric groups or betaine groups, anionic groups, such as carboxylates, thioglycolates, sulfosuccinates, thiosulfates, phosphates and sulfates, this list of course in no way being restrictive (so-called ‘organo-modified’ silicones).

For the purposes of the present invention, the term ‘silicones’ is also intended to include and cover the silanes and in particular the alkylsilanes required for their preparation.

The silicones which are suitable for the present invention and which can be used for sheathing the UV-protection agents are preferably selected from alkylsilanes, polydialkylsiloxanes and polyalkylhydrogenosiloxanes. The silicones are more preferably selected from octyltrimethylsilane, polydimethylsiloxanes and polymethylhydrogenosiloxanes.

The UV-protection agents may be present in the compositions according to the invention in amounts which are generally in the range from 0.15% to 50% by weight and preferably in amounts which are in the range from 0.5% to 20% by weight, where these amounts are based on the total weight of the composition.

In a further, likewise preferred embodiment of the present invention, the preparation according to the invention comprises at least one self-tanning agent.

Advantageous self-tanning agents which can be employed are, inter alia:

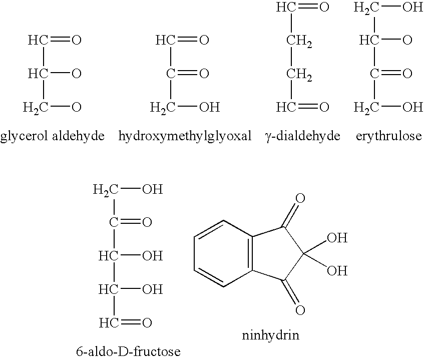

Mention should also be made of 5-hydroxy-1,4-naphthoquinone (juglone), which is extracted from the shells of fresh walnuts

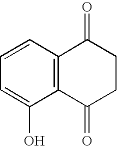

5-hydroxy-1,4-naphthoquinone (juglone)

and 2-hydroxy-1,4-naphthoquinone (lawsone), which occurs in henna leaves

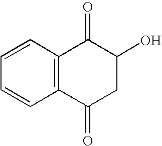

2-hydroxy-1,4-naphthoquinone (lawsone).

Very particular preference is given to 1,3-dihydroxyacetone (DHA), a trifunctional sugar which occurs in the human body, and derivatives thereof.

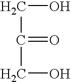

1,3-dihydroxyacetone (DHA).

The present invention thus furthermore relates to the use of antimicrobial compositions according to the invention in combination with self-tanning agents, in particular dihydroxyacetone or dihydroxyacetone derivatives.

Furthermore, the preparations according to the invention may also comprise dyes and coloured pigments that in general do not show any antimicrobial activity. The dyes and coloured pigments can for example be selected from the corresponding positive list in the German Cosmetics Regulation or the EU list of cosmetic colorants. In most cases, they are identical with the dyes approved for foods. Advantageous coloured pigments are, for example, titanium dioxide, mica, iron oxides (for example Fe₂O₃, Fe₃O₄, FeO(OH)) and/or tin oxide. Advantageous dyes are, for example, carmine, Berlin Blue, Chromium Oxide Green, Ultramarine Blue and/or Manganese Violet. It is particularly advantageous to select the dyes and/or coloured pigments from the following list. The Colour Index numbers (CINs) are taken from the Rowe Colour Index, 3rd Edition, Society of Dyers and Colourists, Bradford, England, 1971.

| Chemical or other name | CIN | Colour |
|---|---|---|
| Pigment Green | 10006 | green |
| Acid Green 1 | 10020 | green |
| 2,4-Dinitrohydroxynaphthalene-7-sulfonic acid | 10316 | yellow |
| Pigment Yellow 1 | 11680 | yellow |
| Pigment Yellow 3 | 11710 | yellow |
| Pigment Orange 1 | 11725 | orange |
| 2,4-Dihydroxyazobenzene | 11920 | orange |
| Solvent Red 3 | 12010 | red |
| 1-(2′-Chloro-4′-nitro-1′-phenylazo)-2- | 12085 | red |
| hydroxynaphthalene | | |
| Pigment Red 3 | 12120 | red |
| Ceres Red; Sudan Red; Fat Red G | 12150 | red |
| Pigment Red 112 | 12370 | red |
| Pigment Red 7 | 12420 | red |
| Pigment Brown 1 | 12480 | brown |
| N-(5-chloro-2,4-dimethoxyphenyl)-4-[[5- | 12490 | red |
| [(diethylamino)-sulfonyl]-2-methoxyphenyl]azo]-3- | | |
| hydroxynaphthalene-2-carboxamide | | |
| Disperse Yellow 16 | 12700 | yellow |
| 1-(4-Sulfo-1-phenylazo)-4-aminobenzene-5- | 13015 | yellow |
| sulfonic acid | | |
| 2,4-Dihydroxy-azobenzene-4′-sulfonic acid | 14270 | orange |
| 2-(2,4-Dimethylphenylazo-5-sulfonyl)-1-hydroxy- | 14700 | red |
| naphthalene-4-sulfonic acid | | |
| 2-(4-Sulfo-1-naphthylazo)-1-naphthol-4-sulfonic acid | 14720 | red |
| 2-(6-Sulfo-2,4-xylylazo)-1-naphthol-5-sulfonic acid | 14815 | red |
| 1-(4′-Sulfophenylazo)-2-hydroxynaphthalene | 15510 | orange |
| 1-(2-Sulfonic acid-4-chloro-5-carboxy-1-phenylazo)-2- | 15525 | red |
| hydroxynaphthalene | | |
| 1-(3-Methylphenylazo-4-sulfonyl)-2- | 15580 | red |
| hydroxynaphthalene | | |
| 1-(4′,(8′)-Sulfonyl)-2-hydroxynaphthalene | 15620 | red |
| 2-Hydroxy-1,2′-azonaphthalene-1′-sulfonic acid | 15630 | red |
| 3-Hydroxy-4-phenylazo-2-naphthylcarboxylic acid | 15800 | red |
| 1-(2-Sulfo-4-methyl-1-phenylazo)-2- | 15850 | red |
| naphthylcarboxylic acid | | |
| 1-(2-Sulfo-4-methyl-5-chloro-1-phenylazo)-2-hydroxy- | 15865 | red |
| naphthalene-3-carboxylic acid | | |
| 1-(2-Sulfo-1-naphthylazo)-2-hydroxynaphthalene-3- | 15880 | red |
| carboxylic acid | | |
| 1-(3-Sulfo-1-phenylazo)-2-naphthol-6-sulfonic acid | 15980 | orange |
| 1-(4-Sulfo-1-phenylazo)-2-naphthol-6-sulfonic acid | 15985 | yellow |
| Allura Red | 16035 | red |
| 1-(4-Sulfo-1-naphthylazo)-2-naphthol-3,6-disulfonic | 16185 | red |
| acid | | |
| Acid Orange 10 | 16230 | orange |
| 1-(4-Sulfo-1-naphthylazo)-2-naphthol-6,8-disulfonic | 16255 | red |
| acid | | |
| 1-(4-Sulfo-1-naphthylazo)-2-naphthol-3,6,8-trisulfonic | 16290 | red |
| acid | | |
| 8-Amino-2-phenylazo-1-naphthol-3,6-disulfonic acid | 17200 | red |
| Acid Red 1 | 18050 | red |
| Acid Red 155 | 18130 | red |
| Acid Yellow 121 | 18690 | yellow |
| Acid Red 180 | 18736 | red |
| Acid Yellow 11 | 18820 | yellow |
| Acid Yellow 17 | 18965 | yellow |
| 4-(4-Sulfo-1-phenylazo)-1-(4-sulfophenyl)-5-hydroxy- | 19140 | yellow |
| pyrazolone-3-carboxylic acid | | |
| Pigment Yellow 16 | 20040 | yellow |
| 2,6-(4′-Sulfo-2″,4″- | 20170 | orange |
| dimethyl)bisphenylazo)-1,3-dihydroxy-benzene | | |
| Acid Black 1 | 20470 | black |
| Pigment Yellow 13 | 21100 | yellow |
| Pigment Yellow 83 | 21108 | yellow |
| Solvent Yellow | 21230 | yellow |
| Acid Red 163 | 24790 | red |
| Acid Red 73 | 27290 | red |
| 2-[4′-(4″-Sulfo-1″-phenylazo)-7′-sulfo- | 27755 | black |
| 1′-naphthylazo]-1-hydroxy-7-aminonaphthalene- | | |
| 3,6-disulfonic acid | | |
| 4-[4″-Sulfo-1″-phenylazo)-7′-sulfo-1′-naphthylazo]-1- | 28440 | black |
| hydroxy-8-acetylaminonaphthalene-3,5-disulfonic acid | | |
| Direct Orange 34, 39, 44, 46, 60 | 40215 | orange |
| Food Yellow | 40800 | orange |
| trans-β-Apo-8′-carotene aldehyde (C₃₀) | 40820 | orange |
| trans-Apo-8′-carotinic acid (C₃₀) ethyl ester | 40850 | orange |
| Canthaxanthine | 40850 | orange |
| Acid Blue 1 | 42045 | blue |
| 2,4-Disulfo-5-hydroxy-4′-4″- | 42051 | blue |
| bis(diethylamino)triphenylcarbinol | | |
| 4-[(-4-N-Ethyl-p-sulfobenzylamino)- | 42053 | green |
| phenyl-(4-hydroxy-2-sulfophenyl)(methylene)- | | |
| 1-(N-ethyl-N-p-sulfobenzyl)-2,5- | | |
| cyclohexadienimine] | | |
| Acid Blue 7 | 42080 | blue |
| (N-Ethyl-p-sulfobenzylamino)phenyl-(2-sulfophenyl)- | 42090 | blue |
| methylene-(N-ethyl-N-p-sulfobenzyl)-Δ^{2,5}- | | |
| cyclohexadienimine | | |
| Acid Green 9 | 42100 | green |
| Diethyldisulfobenzyldi-4-amino-2-chlorodi-2-methyl- | 42170 | green |
| fuchsonimmonium | | |
| Basic Violet 14 | 42510 | violet |
| Basic Violet 2 | 42520 | violet |
| 2′-Methyl-4′-(N-ethyl-N-m-sulfobenzyl)amino-4″-(N- | 42735 | blue |
| diethyl)-amino-2-methyl-N-ethyl-N-m- | | |
| sulfobenzylfuchsonimmonium | | |
| 4′-(N-Dimethyl)amino-4″-(N-phenyl)aminonaphtho-N- | 44045 | blue |
| dimethylfuchsonimmonium | | |
| 2-Hydroxy-3,6-disulfo-4,4′-bisdimethylaminonaphtho- | 44090 | green |
| fuchsonimmonium | | |
| Acid Red 52 | 45100 | red |
| 3-(2′-Methylphenylamino)-6-(2′-methyl-4′- | 45190 | violet |
| sulfophenylamino)-9-(2″- | | |
| carboxyphenyl)xanthenium salt | | |
| Acid Red 50 | 45220 | red |
| Phenyl-2-oxyfluorone-2-carboxylic acid | 45350 | yellow |
| 4,5-Dibromofluorescein | 45370 | orange |
| 2,4,5,7-Tetrabromofluorescein | 45380 | red |
| Solvent Dye | 45396 | orange |
| Acid Red 98 | 45405 | red |
| 3′,4′,5′,6′-Tetrachloro-2,4,5,7-tetrabromofluorescein | 45410 | red |
| 4,5-Diiodofluorescein | 45425 | red |
| 2,4,5,7-Tetraiodofluorescein | 45430 | red |
| Quinophthalone | 47000 | yellow |
| Quinophthalonedisulfonic acid | 47005 | yellow |
| Acid Violet 50 | 50325 | violet |
| Acid Black 2 | 50420 | black |
| Pigment Violet 23 | 51319 | violet |
| 1,2-Dioxyanthraquinone, calcium aluminium complex | 58000 | red |
| 3-Oxypyrene-5,8,10-sulfonic acid | 59040 | green |
| 1-Hydroxy-4-N-phenylaminoanthraquinone | 60724 | violet |
| 1-Hydroxy-4-(4′-methylphenylamino)anthraquinone | 60725 | violet |
| Acid Violet 23 | 60730 | violet |
| 1,4-Di(4′-methylphenylamino)anthraquinone | 61565 | green |
| 1,4-Bis(o-sulfo-p-toluidino)anthraquinone | 61570 | green |
| Acid Blue 80 | 61585 | blue |
| Acid Blue 62 | 62045 | blue |
| N,N′-Dihydro-1,2,1′,2′-anthraquinonazine | 69800 | blue |
| Vat Blue 6; Pigment Blue 64 | 69825 | blue |
| Vat Orange 7 | 71105 | orange |
| Indigo | 73000 | blue |
| Indigodisulfonic acid | 73015 | blue |
| 4,4′-Dimethyl-6,6′-dichlorothioindigo | 73360 | red |
| 5,5′-Dichloro-7,7′-dimethylthioindigo | 73385 | violet |
| Quinacridone Violet 19 | 73900 | violet |
| Pigment Red 122 | 73915 | red |
| Pigment Blue 16 | 74100 | blue |
| Phthalocyanine | 74160 | blue |
| Direct Blue 86 | 74180 | blue |
| Chlorinated phthalocyanines | 74260 | green |
| Natural Yellow 6, 19; Natural Red 1 | 75100 | yellow |
| Bixin, Nor-Bixin | 75120 | orange |
| Lycopin | 75125 | yellow |
| trans-alpha-, beta- or gamma-carotene | 75130 | orange |
| Keto and/or hydroxy derivatives of carotene | 75135 | yellow |
| Guanine or pearlescent agent | 75170 | white |
| 1,7-Bis(4-hydroxy-3-methoxyphenyl)- | 75300 | yellow |
| 1,6-heptadiene-3,5-dione | | |
| Complex salt (Na, Al, Ca) of carminic acid | 75470 | red |
| Chlorophyll a and b; copper compounds | 75810 | green |
| of chlorophylls and chlorophyllines | | |
| Aluminium | 77000 | white |
| Aluminium hydroxide | 77002 | white |
| Water-containing aluminium silicates | 77004 | white |
| Ultramarine | 77007 | blue |
| Pigment Red 101 and 102 | 77015 | red |
| Barium sulfate | 77120 | white |
| Bismuth oxychloride and mixtures thereof with mica | 77163 | white |
| Calcium carbonate | 77220 | white |
| Calcium sulfate | 77231 | white |
| Carbon | 77266 | black |
| Pigment Black 9 | 77267 | black |
| Carbo medicinalis vegetabilis | 77268:1 | black |
| Chromium oxide | 77288 | green |
| Chromium oxide, water-containing | 77278 | green |
| Pigment Blue 28, Pigment Green 14 | 77346 | green |
| Pigment Metal 2 | 77400 | brown |
| Gold | 77480 | brown |
| Iron oxides and hydroxides | 77489 | orange |
| Iron oxide | 77491 | red |
| Iron oxide hydrate | 77492 | yellow |
| Iron oxide | 77499 | black |
| Mixtures of iron(II) and iron(III) hexacyanoferrate | 77510 | blue |
| Pigment White 18 | 77713 | white |
| Manganese ammonium diphosphate | 77742 | violet |
| Manganese phosphate; Mn₃(PO₄)₂•7H₂O | 77745 | red |
| Silver | 77820 | white |
| Titanium dioxide and mixtures thereof with mica | 77891 | white |
| Zinc oxide | 77947 | white |
| 6,7-Dimethyl-9-(1′-D-ribityl)isoalloxazine, lactoflavin | | yellow |
| Sugar dye | | brown |
| Capsanthin, capsorubin | | orange |
| Betanin | | red |
| Benzopyrylium salts, anthocyans | | red |
| Aluminium, zinc, magnesium and calcium stearate | | white |
| Bromothymol Blue | | blue |

It may furthermore be favourable to select, as dye, one or more substances from the following group:

2,4-dihydroxyazobenzene, 1-(2′-chloro-4′-nitro-1′-phenylazo)-2-hydroxy-naphthalene, Ceres Red, 2-(4-sulfo-1-naphthylazo)-1-naphthol-4-sulfonic acid, the calcium salt of 2-hydroxy-1,2′-azonaphthalene-1′-sulfonic acid, the calcium and barium salts of 1-(2-sulfo-4-methyl-1-phenylazo)-2-naphthyl-carboxylic acid, the calcium salt of 1-(2-sulfo-1-naphthylazo)-2-hydroxy-naphthalene-3-carboxylic acid, the aluminium salt of 1-(4-sulfo-1-phenylazo)-2-naphthol-6-sulfonic acid, the aluminium salt of 1-(4-sulfo-1-naphthylazo)-2-naphthol-3,6-disulfonic acid, 1-(4-sulfo-1-naphthylazo)-2-naphthol-6,8-disulfonic acid, the aluminium salt of 4-(4-sulfo-1-phenylazo)-2-(4-sulfophenyl)-5-hydroxypyrazolone-3-carboxylic acid, the aluminium and zirconium salts of 4,5-dibromofluorescein, the aluminium and zirconium salts of 2,4,5,7-tetrabromofluorescein, 3′,4′,5′,6′-tetrachloro-2,4,5,7-tetrabromofluorescein and its aluminium salt, the aluminium salt of 2,4,5,7-tetraiodofluorescein, the aluminium salt of quinophthalonedisulfonic acid, the aluminium salt of indigodisulfonic acid, red and black iron oxide (CIN: 77 491 (red) and 77 499 (black)), iron oxide hydrate (CIN: 77492), manganese ammonium diphosphate and titanium dioxide.

Also advantageous are oil-soluble natural dyes, such as, for example, paprika extract, β-carotene or cochineal.

Also advantageous for the purposes of the present invention are gel creams comprising effect pigments. Particular preference is given to the types of effect pigment listed below:
1. Natural effect pigments, such as, for example,
   a) “pearl essence” (guanine/hypoxanthine mixed crystals from fish scales) and
   b) “mother of pearl” (ground mussel shells)
2. Monocrystalline effect pigments, such as, for example, bismuth oxy-chloride (BiOCl)
3. Layered substrate pigments: for example mica/metal oxide

The basis for effect pigments is formed by, for example, pulverulent pigments or castor oil dispersions of bismuth oxychloride and/or titanium dioxide as well as bismuth oxychloride and/or titanium dioxide on mica. The lustre pigment listed under CIN 77163, for example, is particularly advantageous.

Also advantageous are, for example, the following effect pigment types based on mica/metal oxide:

| | Coating/layer | |
|---|---|---|
| Group | thickness | Colour |
| Silver-white effect pigments | TiO₂: 40-60 nm | silver |
| Interference pigments | TiO₂: 60-80 nm | yellow |
| | TiO₂: 80-100 nm | red |
| | TiO₂: 100-140 nm | blue |
| | TiO₂: 120-160 nm | green |
| Coloured lustre pigments | Fe₂O₃ | bronze |
| | Fe₂O₃ | copper |
| | Fe₂O₃ | red |
| | Fe₂O₃ | red-violet |
| | Fe₂O₃ | red-green |
| | Fe₂O₃ | black |
| Combination pigments | TiO₂/Fe₂O₃ | gold shades |
| | TiO₂/Cr₂O₃ | green |
| | TiO₂/Berlin Blue | dark blue |

Particular preference is given to, for example, the pearlescent pigments available from Merck KGaA under the trade names Timiron®, Colorona® or Dichrona®.

The list of the said effect pigments is of course not intended to be limiting. Effect pigments which are advantageous for the purposes of the present invention can be obtained by numerous routes known per se. In addition, other substrates apart from mica can also, for example, be coated with further metal oxides, such as, for example, silica and the like. For example, TiO₂- and Fe₂O₃-coated SiO₂ particles (“Ronasphere” grades), which are marketed by Merck KGaA and are particularly suitable for the optical reduction of fine wrinkles, are advantageous.

It may additionally be advantageous to completely omit a substrate such as mica. Particular preference is given to effect pigments prepared using SiO₂. Such pigments, which may additionally also have goniochromatic effects, are available, for example, from BASF under the trade name Sicopearl® Fantastico.

It may also be advantageous to employ Engelhard pigments based on calcium sodium borosilicate coated with titanium dioxide. These are available under the name Reflecks®. Due to their particle size of 40-80 μm, they have a glitter effect in addition to the colour.

Also particularly advantageous are effect pigments available from Flora Tech under the trade name Metasomes® Standard/Glitter in various colours (yellow, red, green and blue). The glitter particles here are in the form of mixtures with various assistants and dyes (such as, for example, the dyes with the colour index (CI) numbers 19140, 77007, 77289 and 77491).

The dyes and pigments can be in individual form or in the form of a mixture and mutually coated with one another, with different colour effects generally being caused by different coating thicknesses. The total amount of dyes and colouring pigments is advantageously selected from the range from, for example, 0.1% by weight to 30% by weight, preferably from 0.5% to 15% by weight, in particular from 1.0% to 10% by weight, in each case based on the total weight of the preparations.

Furthermore it is preferred to combine compositions according to the present invention with antioxidant properties of antioxidants. Another subject-matter of the present invention is therefore a preparation having antioxidant properties comprising at least one antioxidant, for example a compound of the formula I as described above. These compounds can be used as antioxidants as well as UV filters.

Preference is therefore also given to preparations comprising at least one compound of the formula I which is characterised in that at least two adjacent radicals of the radicals R¹ to R⁴ are OH and at least two adjacent radicals of the radicals R⁵ to R⁷ are OH.

Particularly preferred preparations comprise at least one compound of the formula I which is characterised in that at least three adjacent radicals of the radicals R¹ to R⁴ are OH, preferably with the radicals R¹ to R³ being OH.

In order that the compounds of the formula I are able to develop their positive action as free-radical scavengers on the skin particularly well, it may be preferred to allow the compounds of the formula I to penetrate into deeper skin layers. Several possibilities are available for this purpose. Firstly, the compounds of the formula I can have an adequate Iipophilicity in order to be able to penetrate through the outer skin layer into epidermal layers. As a further possibility, corresponding transport agents, for example liposomes, which enable transport of the compounds of the formula I through the outer skin layers may also be provided in the preparation. Finally, systemic transport of the compounds of the formula I is also conceivable. The preparation is then designed, for example, in such a way that it is suitable for oral administration.

In general, the substances of the formula I act as free-radical scavengers. Free radicals of this type are not generated only by sunlight, but instead are formed under various conditions. Examples are anoxia, which blocks the flow of electrons upstream of the cytochrome oxidases and causes the formation of superoxide free-radical anions; inflammation associated, inter alia, with the formation of superoxide anions by the membrane NADPH oxidase of the leucocytes, but also associated with the formation (through disproportionation in the presence of iron(II) ions) of the hydroxyl free radicals and other reactive species which are normally involved in the phenomenon of phagocytosis; and lipid autooxidation, which is generally initiated by a hydroxyl free radical and produces lipidic alkoxy free radicals and hydroperoxides.

It is assumed that the preferred compounds of the formula I also act as enzyme inhibitors. They presumably inhibit histidine decarboxylase, protein kinases, elastase, aldose reductase and hyaluronidase, and therefore enable the intactness of the basic substance of vascular sheaths to be maintained. Furthermore, they presumably inhibit non-specifically catechol O-methyl transferase, causing the amount of available catecholamine and thus the vascular strength to be increased. Furthermore, they inhibit AMP phosphodiesterase, giving the substances potential for inhibiting thrombocyte aggregation.

Owing to these properties, the preparations according to the invention are, in general, suitable for immune protection and for the protection of DNA and RNA. In particular, the preparations are suitable for the protection of DNA and RNA against oxidative attack, against free radicals and against damage due to radiation, in particular UV radiation. A further advantage of the preparations according to the invention is cell protection, in particular protection of Langerhans cells against damage due to the above-mentioned influences. All these uses and the use of the compounds of the formula I for the preparation of preparations which can be employed correspondingly are expressly also a subject-matter of the present invention.

Of the phenols having an antioxidative action, the polyphenols, some of which are naturally occurring, are of particular interest for applications in the pharmaceutical, cosmetic or nutrition sector. For example, the flavonoids or bioflavonoids, which are principally known as plant dyes, frequently have an antioxidant potential. K. Lemanska, H. Szymusiak, B. Tyrakowska, R. Zielinski, I. M. C. M. Rietjens; Current Topics in Biophysics 2000, 24(2), 101-108, are concerned with effects of the substitution pattern of mono- and dihydroxyflavones. It is observed therein that dihydroxyflavones containing an OH group adjacent to the keto function or OH groups in the 3′,4′- or 6,7- or 7,8-position have antioxidative properties, while other mono- and dihydroxyflavones in some cases do not have antioxidative properties.

Quercetin (cyanidanol, cyanidenolon 1522, meletin, sophoretin, ericin, 3,3′,4′,5,7-pentahydroxyflavone) is frequently mentioned as a particularly effective antioxidant (for example C. A. Rice-Evans, N. J. Miller, G. Paganga, Trends in Plant Science 1997, 2(4), 152-159). K. Lemanska, H. Szymusiak, B. Tyrakowska, R. Zielinski, A. E. M. F. Soffers, I. M. C. M. Rietjens; Free Radical Biology&Medicine 2001, 31(7), 869-881, have investigated the pH dependence of the antioxidant action of hydroxyflavones. Quercetin exhibits the greatest activity amongst the structures investigated over the entire pH range.

For the purposes of the invention, the term flavone derivatives is taken to mean flavonoids and coumaranones. For the purposes of the invention, the term flavonoids is taken to mean the glycosides of flavonones, flavones, 3-hydroxyflavones (=flavonols), aurones, isoflavones and rotenoids [Römpp Chemie Lexikon [Römpp's Lexicon of Chemistry], Volume 9, 1993]. For the purposes of the present invention, however, this is also taken to mean the aglycones, i.e. the sugar-free constituents, and flavonoid and aglycone derivatives. For the purposes of the present invention, the term flavonoid is furthermore also taken to mean anthocyanidine (cyanidine). For the purposes of the present invention, the term coumaranones is also taken to mean derivatives thereof.

Preferred flavonoids are derived from flavonones, flavones, 3-hydroxy-flavones, aurones and isoflavones, in particular from flavonones, flavones, 3-hydroxyflavones and aurones.

The flavonoids are preferably selected from the following compounds: 4,6,3′,4′-tetrahydroxyaurone, quercetin, rutin, isoquercetin, eriodictyol, taxifolin, luteolin, trishydroxyethylquercetin (troxequercetin), trishydroxyethylrutin (troxerutin), trishydroxyethylisoquercetin (troxeisoquercetin), trishydroxyethylluteolin (troxeluteolin), α-glycosylrutin, tiliroside and sulfates and phosphates thereof. Of the flavonoids, particular preference is given to rutin, tiliroside, α-glycosylrutin and troxerutin as active compounds according to the invention.

Of the coumaranones, 4,6,3′,4′-tetrahydroxybenzyl-3-coumaranone is preferred.

The term chromone derivatives is preferably taken to mean certain chromen-2-one derivatives which are suitable as active ingredients for the preventative treatment of human skin and human hair against ageing processes and harmful environmental influences. At the same time, they exhibit a low irritation potential for the skin, have a positive effect on water binding in the skin, maintain or increase the elasticity of the skin and thus promote smoothing of the skin. These compounds preferably conform to the formula II

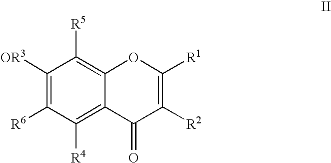

where

R¹ and R² may be identical or different and are selected from
H, —C(═O)—R⁷, —C(═O)—OR⁷,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group can be bonded to a primary or secondary carbon atom in the chain and furthermore the alkyl chain may also be interrupted by oxygen, and/or
C₃- to C₁₀-cycloalkyl groups and/or C₃- to C₁₂-cycloalkenyl groups, where the rings may each also be bridged by —(CH₂)ₙ— groups, where n=1 to 3,

R³ is H or straight-chain or branched C₁- to C₂₀-alkyl groups,

R⁴ is H or OR⁸,

R⁵ and R⁶ may be identical or different and are selected from
—H, —OH,
straight-chain or branched C₁- to C₂₀-alkyl groups,
straight-chain or branched C₃- to C₂₀-alkenyl groups,
straight-chain or branched C₁- to C₂₀-hydroxyalkyl groups, where the hydroxyl group can be bonded to a primary or secondary carbon atom in the chain and furthermore the alkyl chain may also be interrupted by oxygen, and

R⁷ is H, straight-chain or branched C₁- to C₂₀-alkyl groups, a polyhydroxy compound, such as preferably an ascorbic acid radical or glycosidic radicals, and

R⁸ is H or straight-chain or branched C₁- to C₂₀-alkyl groups, where at least 2 of the substituents R¹, R² and R⁴-R⁶ are not H or at least one substituent from R¹ and R² is —C(═O)—R⁷ or —C(═O)—OR⁷.

The proportion of one or more compounds selected from flavonoids, chromone derivatives and coumaranones in the preparation according to the invention is preferably from 0.001% to 5% by weight, particularly preferably from 0.01% to 2% by weight, based on the preparation as a whole.

As already described, preferred compositions according to the invention are also suitable for the treatment of skin diseases associated with a defect in keratinisation which affects differentiation and cell proliferation, in particular for the treatment of acne vulgaris, acne comedonica, polymorphic acne, acne rosaceae, nodular acne, acne conglobata, age-induced acne, acne which arises as a side effect, such as acne solaris, medicament-induced acne or acne professionalis, for the treatment of other defects in keratinisation, in particular ichthyosis, ichthyosiform states, Darier's disease, keratosis palmoplantaris, leucoplasia, leucoplasiform states, herpes of the skin and mucous membrane (buccal) (lichen), for the treatment of other skin diseases associated with a defect in keratinisation and which have an inflammatory and/or immunoallergic component and in particular all forms of psoriasis which affect the skin, mucous membranes and fingers and toenails, and psoriatic rheumatism and skin atopia, such as eczema or respiratory atopia, or hypertrophy of the gums, it furthermore being possible for the compounds to be used for some inflammations which are not associated with a defect in keratinisation, for the treatment of all benign or malignant excrescence of the dermis or epidermis, which may be of viral origin, such as verruca vulgaris, verruca plana, epidermodysplasia verruciformis, oral papillomatosis, papillomatosis florida, and excrescence which may be caused by UV radiation, in particular epithelioma baso-cellulare and epithelioma spinocellulare, for the treatment of other skin diseases, such as dermatitis bullosa and diseases affecting the collagen, for the treatment of certain eye diseases, in particular corneal diseases, for overcoming or combating light-induced skin ageing associated with ageing, for reducing pigmentation and keratosis actinica and for the treatment of all diseases associated with normal ageing or light-induced ageing, for the prevention or healing of wounds/scars of atrophia of the epidermis and/or dermis caused by locally or systemically applied corticosteroids and all other types of skin atrophia, for the prevention or treatment of defects in wound healing, for the prevention or elimination of stretch marks caused by pregnancy or for the promotion of wound healing, for combating defects in tallow production, such as hyperseborrhoea in acne or simple seborrhoea, for combating or preventing cancer-like states or pre-carcinogenic states, in particular promyelocytic leukaemia, for the treatment of inflammatory diseases, such as arthritis, for the treatment of all virus-induced diseases of the skin or other areas of the body, for the prevention or treatment of alopecia, for the treatment of skin diseases or diseases of other areas of the body with an immunological component, for the treatment of cardiovascular diseases, such as arteriosclerosis or hypertension, and of non-insulin-dependent diabetes, and for the treatment of skin problems caused by UV radiation.

The protective action against oxidative stress or against the effect of free radicals can thus be further improved if the preparations comprise one or more further antioxidants. The person skilled in the art being presented with absolutely no difficulties in selecting suitably fast-acting or time-delayed antioxidants.

In a preferred embodiment of the present invention, the preparation is therefore a preparation for the protection of body cells against oxidative stress, in particular for reducing skin ageing, characterised in that it preferably comprises one or more further antioxidants besides the one or more compounds of the formula I.

There are many proven substances known from the specialist literature which also can be used as antioxidants, for example amino acids (for example glycine, histidine, tyrosine, tryptophan) and derivatives thereof, imidazoles (for example urocanic acid) and derivatives thereof, peptides, such as D,L-carnosine, D-carnosine, L-carnosine and derivatives thereof (for example anserine), carotinoids, carotenes (for example α-carotene, β-carotene, lycopene) and derivatives thereof, chlorogenic acid and derivatives thereof, lipoic acid and derivatives thereof (for example dihydrolipoic acid), aurothioglucose, propylthiouracil and other thiols (for example thioredoxin, glutathione, cysteine, cystine, cystamine and the glycosyl, N-acetyl, methyl, ethyl, propyl, amyl, butyl and lauryl, palmitoyl, oleyl, γ-linoleyl, cholesteryl and glyceryl esters thereof) and salts thereof, dilauryl thiodipropionate, distearyl thiodipropionate, thiodipropionic acid and derivatives thereof (esters, ethers, peptides, lipids, nucleotides, nucleosides and salts), and sulfoximine compounds (for example buthionine sulfoximines, homocysteine sulfoximine, buthionine sulfones, penta-, hexa- and heptathionine sulfoximine) in very low tolerated doses (for example pmol to μmol/kg), and also (metal) chelating agents (for example α-hydroxy fatty acids, palmitic acid, phytic acid, lactoferrin), α-hydroxy acids (for example citric acid, lactic acid, malic acid), humic acid, bile acid, bile extracts, bilirubin, biliverdin, EDTA, EGTA and derivatives thereof, unsaturated fatty acids and derivatives thereof, vitamin C and derivatives (for example ascorbyl palmitate, magnesium ascorbyl phosphate, ascorbyl acetate), tocopherols and derivatives (for example vitamin E acetate), vitamin A and derivatives (for example vitamin A palmitate), and coniferyl benzoate of benzoin resin, rutinic acid and derivatives thereof, α-glycosyl rutin, ferulic acid, furfurylideneglucitol, carnosine, butylhydroxytoluene, butylhydroxyanisole, nordihydroguaiaretic acid, trihydroxybutyrophenone, quercetin, uric acid and derivatives thereof, mannose and derivatives thereof, zinc and derivatives thereof (for example ZnO, ZnSO₄), selenium and derivatives thereof (for example selenomethionine), stilbenes and derivatives thereof (for example stilbene oxide, trans-stilbene oxide).

Mixtures of antioxidants are likewise suitable for use in the cosmetic preparations according to the invention. Known and commercial mixtures are, for example, mixtures comprising, as active ingredients, lecithin, L-(+)-ascorbyl palmitate and citric acid (for example Oxynex® AP), natural tocopherols, L-(+)-ascorbyl palmitate, L-(+)-ascorbic acid and citric acid (for example Oxynex® K LIQUID), tocopherol extracts from natural sources, L-(+)-ascorbyl palmitate, L-(+)-ascorbic acid and citric acid (for example Oxynex® L LIQUID), DL-α-tocopherol, L-(+)-ascorbyl palmitate, citric acid and lecithin (for example Oxynex® LM) or butylhydroxytoluene (BHT), L-(+)-ascorbyl palmitate and citric acid (for example Oxynex® 2004). Antioxidants of this type are usually employed with compounds of the formula I in compositions of this type in ratios in the range from 1000:1 to 1:1000, preferably in amounts of from 100:1 to 1:100.

Further antioxidants are compounds as described in WO2006/111233, the disclosure of it being incorporated by reference. In particular, these antioxidants are those of general formula III

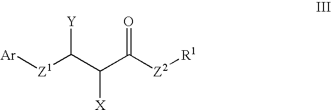

wherein

Ar stands for an unsubstituted or for an single or multiple substituted aromatic ring or condensed ring systems with 6 to 18 C-Atoms, of which at least on ring is aromatic, wherein pro ring also one or two CH-groups can be substituted by C═O, N, O, or S and in one condensed ring system also one or two CH₂-groups can be substituted by C═O or C═CH₂,

R¹ stands for H or a branched or unbranched C₁₋₃₀-alkyl- or C₁₋₃₀-hydroxyalkylgroup or an group R^{a} or R^{b}

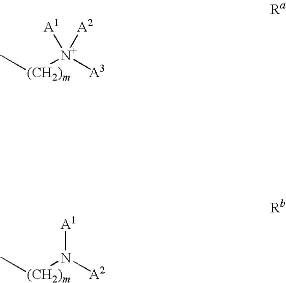

where m stands for an integer in the range of 1 to 30 and A¹-A³ are independently of each other an benzyl group or a —(CH₂O)ₙ(CH₂)ₒ(O)ₚH group, wherein m and o are each, independently of one another an integer in the range of 0 to 30 and p stands for 0 or 1,

X stands for a group selected of —H, —CN, —C(═O)—R¹ und —C(═O)—Z²—R¹,

Y means H or Ar,

Z¹ and Z² are each, independently of one another O, S, CR⁷R⁸, NR⁷ or a single bond,

R⁷ and R⁸ are each, independently of one another selected of H, OH, unbranched or branched C₁- to C₂₀-alkoxy groups, unbranched or branched C₁- to C₂₀-alkyl groups, unbranched or branched C₃- to C₂₀-alkenyl groups, unbranched or branched C₁- to C₂₀-hydroxyalkyl groups, where the hydroxy group is bound to an primary or secondary carbon atom of the chain and where the alkyl chain can be intermitted by oxygen, unbranched or branched C₁- to C₂₀-hydroxyalkoxy groups, where the hydroxyl group(s) can be bound to an primary or secondary carbon atom of the chain and the alkyl chain can be intermitted by oxygen, and salts of compounds of formula III respectively.

Preferred compounds of formula III are compounds of formula B,

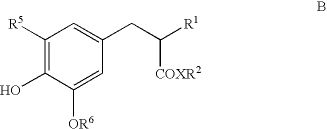

in which

R¹ is selected from the group comprising —C(O)CH₃, —CO₂R³, —C(O)NH₂ and —C(O)N(R⁴)₂,

X is O or NH,

R² is linear or branched alkyl with 1 to 30 C-atoms,

R³ is linear or branched alkyl with 1 to 20 C-atoms,

R⁴ is independently of each other H or linear or branched alkyl with 1 to 8 C-atoms,

R⁵ is linear or branched alkyl with 1 to 8 C-atoms or linear or branched alkoxy with 1 to 8 C-atoms and

R⁶ is linear or branched alkyl with 1 to 8 C-atoms, preferably derivatives of 2-(4-hydroxy-3,5-dimethoxybenzyl)-malonic acid, particularly preferably bis-(2-ethylhexyl) 2-(4-hydroxy-3,5-dimethoxybenzyl)-malonate (e.g. RonaCare® AP).

The preparations according to the invention may comprise vitamins as further ingredients. The preparations according to the invention preferably comprise vitamins and vitamin derivatives selected from vitamin A, vitamin A propionate, vitamin A palmitate, vitamin A acetate, retinol, vitamin B, thiamine chloride hydrochloride (vitamin B₁), riboflavin (vitamin B₂), nicotinamide, vitamin C (ascorbic acid), vitamin D, ergocalciferol (vitamin D₂), vitamin E, DL-α-tocopherol, tocopherol E acetate, tocopherol hydrogensuccinate, vitamin K₁, esculin (vitamin P active ingredient), thiamine (vitamin B₁), nicotinic acid (niacin), pyridoxine, pyridoxal, pyridoxamine, (vitamin B₆), pantothenic acid, biotin, folic acid and cobalamine (vitamin B₁₂), particularly preferably vitamin A palmitate, vitamin C and derivatives thereof, DL-α-tocopherol, tocopherol E acetate, nicotinic acid, pantothenic acid and biotin. Vitamins are usually employed here with compounds of the formula I in ratios in the range from 1000:1 to 1:1000, preferably in amounts of from 100:1 to 1:100.

The preparations according to the invention may in addition comprise further conventional skin-protecting or skin-care active ingredients. These may in principle be any active ingredients known to the person skilled in the art.

Particularly preferred active ingredients are pyrimidinecarboxylic acids and/or aryl oximes.

Pyrimidinecarboxylic acids occur in halophilic microorganisms and play a role in osmoregulation of these organisms (E. A. Galinski et al., Eur. J. Biochem., 149 (1985) pages 135-139). Of the pyrimidinecarboxylic acids, particular mention should be made here of ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid) and derivatives thereof. These compounds stabilise enzymes and other biomolecules in aqueous solutions and organic solvents. Furthermore, they stabilise, in particular, enzymes against denaturing conditions, such as salts, extreme pH values, surfactants, urea, guanidinium chloride and other compounds.

Ectoin and ectoin derivatives, such as hydroxyectoin, can advantageously be employed in medicaments. In particular, hydroxyectoin can be employed for the preparation of a medicament for the treatment of skin diseases. Other areas of application of hydroxyectoin and other ectoin derivatives are typically in areas in which, for example, trehalose is used as additive. Thus, ectoin derivatives, such as hydroxyectoin, can be used as protectant in dried yeast and bacteria cells. Pharmaceutical products, such as non-glycosylated, pharmaceutically active peptides and proteins, for example t-PA, can also be protected with ectoin or its derivatives.

Of the cosmetic applications, particular mention should be made of the use of ectoin and ectoin derivatives for the care of aged, dry or irritated skin. Thus, European Patent Application EP-A-0 671 161 describes, in particular, that ectoin and hydroxyectoin are employed in cosmetic preparations, such as powders, soaps, surfactant-containing cleansing products, lipsticks, rouge, make-ups, care creams and sunscreen preparations.

Preference is given here to the use of a pyrimidinecarboxylic acid of the following formula IV

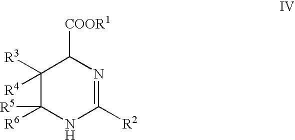

in which R¹ is a radical H or C1-8-alkyl, R² is a radical H or C1-4-alkyl, and R³, R⁴, R⁵ and R⁶ are each, independently of one another, a radical from the group consisting of H, OH, NH₂ and C1-4-alkyl. Preference is given to the use of pyrimidinecarboxylic acids in which R² is a methyl or ethyl group, and R¹ or R⁵ and R⁶ are H. Particular preference is given to the use of the pyrimidinecarboxylic acids ectoin ((S)-1,4,5,6-tetrahydro-2-methyl-4-pyrimidinecarboxylic acid) and hydroxyectoin ((S,S)-1,4,5,6-tetrahydro-5-hydroxy-2-methyl-4-pyrimidinecarboxylic acid). The preparations according to the invention preferably comprise pyrimidinecarboxylic acids of this type in amounts of up to 15% by weight. In combination with compounds of formula I, the pyrimidinecarboxylic acids are preferably employed in ratios of from 100:1 to 1:100 with respect to the compounds of the formula I, with ratios in the range from 1:10 to 10:1 being particularly preferred.

Of the aryl oximes, preference is given to the use of 2-hydroxy-5-methyl-laurophenone oxime, which is also known as HMLO, LPO or F5. Its suitability for use in cosmetic compositions is disclosed, for example, in DE-A-41 16 123. Preparations which comprise 2-hydroxy-5-methyllaurophenone oxime are accordingly suitable for the treatment of skin diseases which are accompanied by inflammation. It is known that preparations of this type can be used, for example, for the therapy of psoriasis, various forms of eczema, irritative and toxic dermatitis, UV dermatitis and further allergic and/or inflammatory diseases of the skin and integumentary appendages. Preparations according to the invention which, in addition to the compound of the formula I, additionally comprise an aryl oxime, preferably 2-hydroxy-5-methyllaurophenone oxime, exhibit surprising antiinflammatory suitability. The preparations here preferably comprise from 0.01% to 10% by weight of the aryl oxime, it being particularly preferred for the preparation to comprise from 0.05% to 5% by weight of aryl oxime.

All compounds or components which can be used in the preparations are either known or are commercially available or can be synthesised by known processes.

Besides the compounds described here, the preparations according to the invention may also comprise at least one photostabiliser, preferably conforming to the formula V

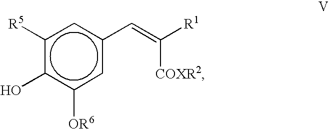

where
R¹ is selected from —C(O)CH₃, —CO₂R³, —C(O)NH₂ and —C(O)N(R⁴)₂;
X is O or NH;
R² is a linear or branched C₁₋₃₀-alkyl radical;
R³ is a linear or branched C₁₋₂₀-alkyl radical,
all R⁴, independently of one another, are H or linear or branched C₁₋₈-alkyl radicals,
R⁵ is H, a linear or branched C₁₋₈-alkyl radical or a linear or branched —O—C₁₋₈-alkyl radical, and

R⁶ is a C₁₋₈-alkyl radical,

where the photostabiliser is particularly preferably bis(2-ethylhexyl) 2-(4-hydroxy-3,5-dimethoxybenzylidene)malonate. Corresponding photostabilisers and their preparation and use are described in International Patent Application WO 03/007906, the disclosure content of which expressly also belongs to the subject-matter of the present application.

The formulations according to the invention can be prepared by processes that are well known to the person skilled in the art, in particular by the processes that serve for the preparation of oil-in-water emulsions or water-in-oil emulsions.

The present invention furthermore relates to preparations having antimicrobial properties comprising the compositions according to the invention and one or more cosmetically or dermatologically suitable vehicles, to a process for the production of a preparation which is characterised in that a composition according to the invention is mixed with a cosmetically or dermatologically suitable vehicle, and to the use of compositions according to the invention for the production of a preparation having antimicrobial properties.

These preparations can be, in particular, in the form of simple or complex emulsions (O/W, W/O, O/W/O or W/O/W), such as creams, milks, gels, or gel-creams, powders and solid sticks, and they may, if desired, be formulated as aerosols and be in the form of foams or sprays.

The cosmetic formulations according to the invention can be used as preparations for protection of the human epidermis or of the hair against UV radiation, as sunscreens or make-up products.

It should be pointed out that in the formulations according to the invention for sun protection which have a vehicle of the oil-in-water emulsion type, the aqueous phase (which comprises, in particular, the hydrophilic filters) generally makes up from 50% to 95% by weight and preferably from 70% to 90% by weight, based on the formulation as a whole, the oil phase (which comprises, in particular, the lipophilic filters) makes up from 5% to 50% by weight and preferably from 10% to 30% by weight, based on the formulation as a whole, and the (co)emulsifier or (co)emulsifiers make(s) up from 0.5% to 20% by weight and preferably from 2% to 10% by weight, based on the formulation as a whole.

For example, the one or more compounds of the formula I can be incorporated into cosmetic or dermatological preparations in the customary manner. Suitable preparations are those for external use, for example in the form of a cream, lotion or gel or as a solution that can be sprayed onto the skin. Suitable for internal use are administration forms such as capsules, coated tablets, powders, tablet solutions or solutions.

Examples which may be mentioned of application forms of the preparations according to the invention are: solutions, suspensions, emulsions, PIT emulsions, pastes, ointments, gels, creams, lotions, powders, soaps, surfactant-containing cleansing preparations, oils, aerosols and sprays. Examples of other application forms are sticks, shampoos and shower preparations. Any desired customary excipients, auxiliaries and, if desired, further active ingredients may be added to the preparation.

Preferred auxiliaries originate from the group consisting of preservatives, antioxidants, stabilisers, solubilisers, vitamins, colorants and odour improvers.

Ointments, pastes, creams and gels may comprise the customary excipients, for example animal and vegetable fats, waxes, paraffins, starch, tragacanth, cellulose derivatives, polyethylene glycols, silicones, bentonites, silica, talc and zinc oxide, or mixtures of these substances.

Powders and sprays may comprise the customary excipients, for example lactose, talc, silica, aluminium hydroxide, calcium silicate and polyamide powder, or mixtures of these substances. Sprays may additionally comprise the customary propellants, for example chlorofluorocarbons, propane/butane or dimethyl ether.

Solutions and emulsions may comprise the customary excipients, such as solvents, solubilisers and emulsifiers, for example water, ethanol, isopropanol, ethyl carbonate, ethyl acetate, benzyl alcohol, benzyl benzoate, propylene glycol, 1,3-butyl glycol, oils, in particular cottonseed oil, peanut oil, wheatgerm oil, olive oil, castor oil and sesame oil, glycerol fatty acid esters, polyethylene glycols and fatty acid esters of sorbitan, or mixtures of these substances.

Suspensions may comprise the customary excipients, such as liquid diluents, for example water, ethanol or propylene glycol, suspending agents, for example ethoxylated isostearyl alcohols, polyoxyethylene sorbitol esters and polyoxyethylene sorbitan esters, microcrystalline cellulose, aluminium metahydroxide, bentonite, agar-agar and tragacanth, or mixtures of these substances.

Soaps may comprise the customary excipients, such as alkali metal salts of fatty acids, salts of fatty acid monoesters, fatty acid protein hydrolysates, isethionates, lanolin, fatty alcohol, vegetable oils, plant extracts, glycerol, sugars, or mixtures of these substances.

Surfactant-containing cleansing products can comprise the conventional carriers, such as salts of fatty alcohol sulfates, fatty alcohol ether sulfates, sulfosuccinic acid monoesters, fatty acid albumen hydrolysates, isothionates, imidazolinium derivatives, methyl taurates, sarcosinates, fatty acid amide ether sulfates, alkylamidobetaines, fatty alcohols, fatty acid glycerides, fatty acid diethanolamides, vegetable and synthetic oils, lanolin derivatives, ethoxylated glycerol fatty acid esters or mixtures of these substances.

Face and body oils may comprise the customary excipients, such as synthetic oils, such as fatty acid esters, fatty alcohols, silicone oils, natural oils, such as vegetable oils and oily plant extracts, paraffin oils or lanolin oils, or mixtures of these substances.

Further typical cosmetic application forms are also lipsticks, lip-care sticks, mascara, eyeliner, eye-shadow, rouge, powder make-up, emulsion make-up and wax make-up, and sunscreen, pre-sun and after-sun preparations.

The preferred preparation forms according to the invention include, in particular, emulsions.

Emulsions according to the invention are advantageous and comprise, for example, the said fats, oils, waxes and other fatty substances, as well as water and an emulsifier, as usually used for a preparation of this type.

The lipid phase may advantageously be selected from the following group of substances:
mineral oils, mineral waxes;
oils, such as triglycerides of capric or caprylic acid, furthermore natural oils, such as, for example, castor oil;
fats, waxes and other natural and synthetic fatty substances, preferably esters of fatty acids with alcohols having a low carbon number, for example with isopropanol, propylene glycol or glycerol, or esters of fatty alcohols with alkanoic acids having a low carbon number or with fatty acids;
silicone oils, such as dimethylpolysiloxanes, diethylpolysiloxanes, diphenylpolysiloxanes and mixed forms thereof.

For the purposes of the present invention, the oil phase of the emulsions, oleogels or hydrodispersions or lipodispersions is advantageously selected from the group consisting of esters of saturated and/or unsaturated, branched and/or unbranched alkanecarboxylic acids having a chain length of from 3 to 30 carbon atoms and saturated and/or unsaturated, branched and/or unbranched alcohols having a chain length of from 3 to 30 carbon atoms, or from the group consisting of esters of aromatic carboxylic acids and saturated and/or unsaturated, branched and/or unbranched alcohols having a chain length of from 3 to 30 carbon atoms. Ester oils of this type can then advantageously be selected from the group consisting of isopropyl myristate, isopropyl palmitate, isopropyl stearate, isopropyl oleate, n-butyl stearate, n-hexyl laurate, n-decyl oleate, isooctyl stearate, isononyl stearate, isononyl isononanoate, 2-ethylhexyl palmitate, 2-ethylhexyl laurate, 2-hexyldecyl stearate, 2-octyldodecyl palmitate, oleyl oleate, oleyl erucate, erucyl oleate, erucyl erucate and synthetic, semi-synthetic and natural mixtures of esters of this type, for example jojoba oil.

The oil phase may furthermore advantageously be selected from the group consisting of branched and unbranched hydrocarbons and waxes, silicone oils, dialkyl ethers, or the group consisting of saturated and unsaturated, branched and unbranched alcohols, and fatty acid triglycerides, specifically the triglycerol esters of saturated and/or unsaturated, branched and/or unbranched alkanecarboxylic acids having a chain length of from 8 to 24, in particular 12-18, carbon atoms. The fatty acid triglycerides may advantageously be selected, for example, from the group consisting of synthetic, semi-synthetic and natural oils, for example olive oil, sunflower oil, soya oil, peanut oil, rapeseed oil, almond oil, palm oil, coconut oil, palm kernel oil and the like.

Any desired mixtures of oil and wax components of this type may also advantageously be employed for the purposes of the present invention. It may also be advantageous to employ waxes, for example cetyl palmitate, as the only lipid component of the oil phase.

The oil phase is advantageously selected from the group consisting of 2-ethylhexyl isostearate, octyldodecanol, isotridecyl isononanoate, iso-eicosane, 2-ethylhexyl cocoate, C₁₂₋₁₅-alkyl benzoate, caprylic/capric acid triglyceride and dicapryl ether.

Particularly advantageous are mixtures of C₁₂₋₁₅-alkyl benzoate and 2-ethyl-hexyl isostearate, mixtures of C₁₂₋₁₅-alkyl benzoate and isotridecyl isononanoate, as well as mixtures of C₁₂₋₁₅-alkyl benzoate, 2-ethylhexyl isostearate and isotridecyl isononanoate.

Of the hydrocarbons, paraffin oil, squalane and squalene may advantageously be used for the purposes of the present invention.

Furthermore, the oil phase may also advantageously have a content of cyclic or linear silicone oils or consist entirely of oils of this type, although it is preferred to use an additional content of other oil-phase components in addition to the silicone oil or the silicone oils.

The silicone oil to be used in accordance with the invention is advantageously cyclomethicone (octamethylcyclotetrasiloxane). However, it is also advantageous for the purposes of the present invention to use other silicone oils, for example hexamethylcyclotrisiloxane, polydimethylsiloxane or poly(methylphenylsiloxane).

Also particularly advantageous are mixtures of cyclomethicone and isotridecyl isononanoate and of cyclomethicone and 2-ethylhexyl isostearate.

The aqueous phase of the preparations according to the invention optionally advantageously comprises alcohols, diols or polyols having a low carbon number, and ethers thereof, preferably ethanol, isopropanol, propylene glycol, glycerol, ethylene glycol, ethylene glycol monoethyl or monobutyl ether, propylene glycol monomethyl, monoethyl or monobutyl ether, diethylene glycol monomethyl or monoethyl ether and analogous products, furthermore alcohols having a low carbon number, for example ethanol, isopropanol, 1,2-propanediol or glycerol, and, in particular, one or more thickeners, which may advantageously be selected from the group consisting of silicon dioxide, aluminium silicates, polysaccharides and derivatives thereof, for example hyaluronic acid, xanthan gum, hydroxypropylmethylcellulose, particularly advantageously from the group consisting of the polyacrylates, preferably a polyacrylate from the group consisting of the so-called Carbopols, for example Carbopol grades 980, 981, 1382, 2984 or 5984, in each case individually or in combination.

In particular, mixtures of the above-mentioned solvents are used. In the case of alcoholic solvents, water may be a further constituent.

Emulsions according to the invention are advantageous and comprise, for example, the said fats, oils, waxes and other fatty substances, as well as water and an emulsifier, as usually used for a formulation of this type.

In a preferred embodiment, the preparations according to the invention comprise hydrophilic surfactants.

The hydrophilic surfactants are preferably selected from the group consisting of the alkylglucosides, acyl lactylates, betaines and coconut amphoacetates.

The alkylglucosides are themselves advantageously selected from the group consisting of the alkylgluosides which are distinguished by the structural formula

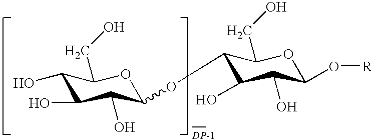

where R is a branched or unbranched alkyl radical having from 4 to 24 carbon atoms, and where D̅P̅ denotes a mean degree of glucosylation of up to 2.

The value D̅P̅ represents the degree of glucosidation of the alkylglucosides used in accordance with the invention and is defined as

$$\overline{DP} = \frac{{p}_{1}}{100} \cdot 1 + \frac{{p}_{2}}{100} \cdot 2 + \frac{{p}_{3}}{100} \cdot 3 + … = \sum \frac{{p}_{i}}{100} \cdot i$$

in which p₁, p₂, p₃ . . . pᵢ represent the proportion of mono-, di-, tri- . . . i-fold glucosylated products in per cent by weight. Advantageous according to the invention are products having degrees of glucosylation of 1-2, particularly advantageously of from 1.1 to 1.5, very particularly advantageously of 1.2-1.4, in particular of 1.3.

The value DP takes into account the fact that alkylglucosides are generally, as a consequence of their preparation, in the form of mixtures of mono- and oligoglucosides. A relatively high content of monoglucosides, typically in the order of 40%-70% by weight, is advantageous in accordance with the invention.

Alkylglycosides which are particularly advantageously used for the pur-poses of the invention are selected from the group consisting of octyl glucopyranoside, nonyl glucopyranoside, decyl glucopyranoside, undecyl glucopyranoside, dodecyl glucopyranoside, tetradecyl glucopyranoside and hexadecyl glucopyranoside.

It is likewise advantageous to employ natural or synthetic raw materials and auxiliaries or mixtures which are distinguished by an effective content of the active ingredients used in accordance with the invention, for example Plantaren® 1200 (Henkel KGaA), Oramix® NS 10 (Seppic).

The acyllactylates are themselves advantageously selected from the group consisting of the substances which are distinguished by the structural formula

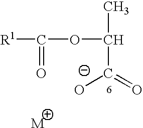

where R¹ is a branched or unbranched alkyl radical having from 1 to 30 carbon atoms, and M⁺ is selected from the group consisting of the alkali metal ions and the group consisting of ammonium ions which are substituted by one or more alkyl and/or one or more hydroxyalkyl radicals, or corresponds to half an equivalent of an alkaline earth metal ion.

For example, sodium isostearyl lactylate, for example the product Pathionic® ISL from the American Ingredients Company, is advantageous.

The betaines are advantageously selected from the group consisting of the substances which are distinguished by the structural formula

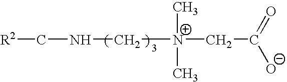

where R² is a branched or unbranched alkyl radical having from 1 to 30 carbon atoms.

R² is particularly advantageously a branched or unbranched alkyl radical having from 6 to 12 carbon atoms.

For example, capramidopropylbetaine, for example the product Tego® Betain 810 from Th. Goldschmidt AG, is advantageous.

A coconut amphoacetate which is advantageous for the purposes of the invention is, for example, sodium coconut amphoacetate, as available under the name Miranol® Ultra C32 from Miranol Chemical Corp.

The preparations according to the invention are advantageously characterised in that the hydrophilic surfactant(s) is (are) present in concentrations of 0.01%-20% by weight, preferably 0.05%-10% by weight, particularly preferably 0.1%-5% by weight, in each case based on the total weight of the composition.

For use, the cosmetic and dermatological preparations according to the invention are applied to the skin and/or the hair in an adequate amount in the usual manner for cosmetics.

Cosmetic and dermatological preparations according to the invention may exist in various forms. Thus, they may be, for example, a solution, a water-free preparation, an emulsion or microemulsion of the water-in-oil (W/O) or oil-in-water (O/W) type, a multiple emulsion, for example of the water-in-oil-in-water (W/O/W) type, a gel, a solid stick, an ointment or an aerosol. It is also advantageous to administer ectoins in encapsulated form, for example in collagen matrices and other conventional encapsulation materials, for example as cellulose encapsulations, in gelatine, wax matrices or liposomally encapsulated. In particular, wax matrices, as described in DE-A 43 08 282, have proven favourable. Preference is given to emulsions. O/W emulsions are particularly preferred. Emulsions, W/O emulsions and O/W emulsions are obtainable in a conventional manner.

Emulsifiers that can be used are, for example, the known W/O and O/W emulsifiers. It is advantageous to use further conventional co-emulsifiers in the preferred O/W emulsions according to the invention. The commercially available product Ceralution C (Sasol) has to be proven to be in particular advantageous as emulsifier.

Co-emulsifiers which are advantageous according to the invention are, for example, O/W emulsifiers, principally from the group consisting of the substances having HLB values of 11-16, very particularly advantageously having HLB values of 14.5-15.5, so long as the O/W emulsifiers have saturated radicals R and R′. If the O/W emulsifiers have unsaturated radicals R and/or R′ or in the case of isoalkyl derivatives, the preferred HLB value of such emulsifiers may also be lower or higher.

It is advantageous to select the fatty alcohol ethoxylates from the group consisting of ethoxylated stearyl alcohols, cetyl alcohols, cetylstearyl alcohols (cetearyl alcohols). Particular preference is given to the following: polyethylene glycol (13) stearyl ether (steareth-13), polyethylene glycol (14) stearyl ether (steareth-14), polyethylene glycol (15) stearyl ether (steareth-15), polyethylene glycol (16) stearyl ether (steareth-16), polyethylene glycol (17) stearyl ether (steareth-17), polyethylene glycol (18) stearyl ether (steareth-18), polyethylene glycol (19) stearyl ether (steareth-19), polyethylene glycol (20) stearyl ether (steareth-20), polyethylene glycol (12) isostearyl ether (isosteareth-12), polyethylene glycol (13) isostearyl ether (isosteareth-13), polyethylene glycol (14) isostearyl ether (isosteareth-14), polyethylene glycol (15) isostearyl ether (isosteareth-15), polyethylene glycol (16) isostearyl ether (isosteareth-16), polyethylene glycol (17) isostearyl ether (isosteareth-17), polyethylene glycol (18) isostearyl ether (isosteareth-18), polyethylene glycol (19) isostearyl ether (isosteareth-19), polyethylene glycol (20) isostearyl ether (isosteareth-20), polyethylene glycol (13) cetyl ether (ceteth-13), polyethylene glycol (14) cetyl ether (ceteth-14), polyethylene glycol (15) cetyl ether (ceteth-15), polyethylene glycol (16) cetyl ether (ceteth-16), polyethylene glycol (17) cetyl ether (ceteth-17), polyethylene glycol (18) cetyl ether (ceteth-18), polyethylene glycol (19) cetyl ether (ceteth-19), polyethylene glycol (20) cetyl ether (ceteth-20), polyethylene glycol (13) isocetyl ether (isoceteth-13), polyethylene glycol (14) isocetyl ether (isoceteth-14), polyethylene glycol (15) isocetyl ether (isoceteth-15), polyethylene glycol (16) isocetyl ether (isoceteth-16), polyethylene glycol (17) isocetyl ether (isoceteth-17), polyethylene glycol (18) isocetyl ether (isoceteth-18), polyethylene glycol (19) isocetyl ether (isoceteth-19), polyethylene glycol (20) isocetyl ether (isoceteth-20), polyethylene glycol (12) oleyl ether (oleth-12), polyethylene glycol (13) oleyl ether (oleth-13), polyethylene glycol (14) oleyl ether (oleth-14), polyethylene glycol (15) oleyl ether (oleth-15), polyethylene glycol (12) lauryl ether (laureth-12), polyethylene glycol (12) isolauryl ether (isolaureth-12), polyethylene glycol (13) cetylstearyl ether (ceteareth-13), polyethylene glycol (14) cetylstearyl ether (ceteareth-14), polyethylene glycol (15) cetylstearyl ether (ceteareth-15), polyethylene glycol (16) cetylstearyl ether (ceteareth-16), polyethylene glycol (17) cetylstearyl ether (ceteareth-17), polyethylene glycol (18) cetylstearyl ether (ceteareth-18), polyethylene glycol (19) cetylstearyl ether (ceteareth-19), polyethylene glycol (20) cetylstearyl ether (ceteareth-20).

It is furthermore advantageous to select the fatty acid ethoxylates from the following group:

polyethylene glycol (20) stearate, polyethylene glycol (21) stearate, polyethylene glycol (22) stearate, polyethylene glycol (23) stearate, polyethylene glycol (24) stearate, polyethylene glycol (25) stearate, polyethylene glycol (12) isostearate, polyethylene glycol (13) isostearate, polyethylene glycol (14) isostearate, polyethylene glycol (15) isostearate, polyethylene glycol (16) isostearate, polyethylene glycol (17) isostearate, polyethylene glycol (18) isostea rate, polyethylene glycol (19) isostearate, polyethylene glycol (20) isostearate, polyethylene glycol (21) isostearate, polyethylene glycol (22) isostearate, polyethylene glycol (23) isostearate, polyethylene glycol (24) isostearate, polyethylene glycol (25) isostearate, polyethylene glycol (12) oleate, polyethylene glycol (13) oleate, polyethylene glycol (14) oleate, polyethylene glycol (15) oleate, polyethylene glycol (16) oleate, polyethylene glycol (17) oleate, polyethylene glycol (18) oleate, polyethylene glycol (19) oleate, polyethylene glycol (20) oleate.

The ethoxylated alkyl ether carboxylic acid or salt thereof used can advantageously be sodium laureth-11 carboxylate. An alkyl ether sulfate which can advantageously be used is sodium laureth-14 sulfate. An ethoxylated cholesterol derivative which can advantageously be used is polyethylene glycol (30) cholesteryl ether. Polyethylene glycol (25) soyasterol has also proven successful. Ethoxylated triglycerides, which can advantageously be used, are the polyethylene glycol (60) evening primrose glycerides.

It is furthermore advantageous to select the polyethylene glycol glycerol fatty acid esters from the group consisting of polyethylene glycol (20) glyceryl laurate, polyethylene glycol (21) glyceryl laurate, polyethylene glycol (22) glyceryl laurate, polyethylene glycol (23) glyceryl laurate, polyethylene glycol (6) glyceryl caprate/caprinate, polyethylene glycol (20) glyceryl oleate, polyethylene glycol (20) glyceryl isostearate, polyethylene glycol (18) glyceryl oleate/cocoate.

It is likewise favourable to select the sorbitan esters from the group consisting of polyethylene glycol (20) sorbitan monolaurate, polyethylene glycol (20) sorbitan monostearate, polyethylene glycol (20) sorbitan monoisostearate, polyethylene glycol (20) sorbitan monopalmitate, polyethylene glycol (20) sorbitan monooleate.

Optional W/O emulsifiers, but ones which may nevertheless be advantageous for the purposes of the invention are the following:

fatty alcohols having from 8 to 30 carbon atoms, monoglycerol esters of saturated and/or unsaturated, branched and/or unbranched alkane-carboxylic acids having a chain length of from 8 to 24 carbon atoms, in particular 12-18 carbon atoms, diglycerol esters of saturated and/or unsaturated, branched and/or unbranched alkanecarboxylic acids having a chain length of from 8 to 24 carbon atoms, in particular 12-18 carbon atoms, monoglycerol ethers of saturated and/or unsaturated, branched and/or unbranched alcohols having a chain length of from 8 to 24 carbon atoms, in particular 12-18 carbon atoms, diglycerol ethers of saturated and/or unsaturated, branched and/or unbranched alcohols having a chain length of from 8 to 24 carbon atoms, in particular 12-18 carbon atoms, propylene glycol esters of saturated and/or unsaturated, branched and/or unbranched alkanecarboxylic acids having a chain length of from 8 to 24 carbon atoms, in particular 12-18 carbon atoms, and sorbitan esters of saturated and/or unsaturated, branched and/or unbranched alkanecarboxylic acids having a chain length of from 8 to 24 carbon atoms, in particular 12-18 carbon atoms.

Particularly advantageous W/O emulsifiers are glyceryl monostearate, glyceryl monoisostearate, glyceryl monomyristate, glyceryl monooleate, diglyceryl monostearate, diglyceryl monoisostearate, propylene glycol monostearate, propylene glycol monoisostearate, propylene glycol monocaprylate, propylene glycol monolaurate, sorbitan monoisostearate, sorbitan monolaurate, sorbitan monocaprylate, sorbitan monoisooleate, sucrose distearate, cetyl alcohol, stearyl alcohol, arachidyl alcohol, behenyl alcohol, isobehenyl alcohol, selachyl alcohol, chimyl alcohol, polyethylene glycol (2) stearyl ether (steareth-2), glyceryl monolaurate, glyceryl monocaprinate and glyceryl monocaprylate.

The preferred preparations according to the invention are particularly suitable for protecting human skin against ageing processes and against oxidative stress, i.e. against damage caused by free radicals, as are produced, for example, by solar irradiation, heat or other influences. In this connection, it is in the various administration forms usually used for this application. For example, it may, in particular, be in the form of a lotion or emulsion, such as in the form of a cream or milk (O/W, W/O, O/W/O, W/O/W), in the form of oily-alcoholic, oily-aqueous or aqueous-alcoholic gels or solutions, in the form of solid sticks or may be formulated as an aerosol.

The preparation may comprise cosmetic adjuvants which are usually used in this type of preparation, such as, for example, thickeners, softeners, moisturisers, surface-active agents, emulsifiers, preservatives, antifoams, perfumes, waxes, lanolin, propellants, dyes and/or pigments which colour the composition itself or the skin, and other ingredients usually used in cosmetics.

The dispersant or solubiliser used can be an oil, wax or other fatty substance, a lower monoalcohol or lower polyol or mixtures thereof. Particularly preferred monoalcohols or polyols include ethanol, isopropanol, propylene glycol, glycerol and sorbitol.

A preferred embodiment of the invention is an emulsion in the form of a protective cream or milk which, apart from the compound(s) of the formula I, comprises, for example, fatty alcohols, fatty acids, fatty acid esters, in particular triglycerides of fatty acids, lanolin, natural and synthetic oils or waxes and emulsifiers in the presence of water.

Further preferred embodiments are oily lotions based on natural or synthetic oils and waxes, lanolin, fatty acid esters, in particular triglycerides of fatty acids, or oily-alcoholic lotions based on a lower alcohol, such as ethanol, or a glycerol, such as propylene glycol, and/or a polyol, such as glycerol, and oils, waxes and fatty acid esters, such as triglycerides of fatty acids.

The preparation according to the invention may also be in the form of an alcoholic gel which comprises one or more lower alcohols or polyols, such as ethanol, propylene glycol or glycerol, and a thickener, such as siliceous earth. The oily-alcoholic gels also comprise natural or synthetic oil or wax.

The solid sticks consist of natural or synthetic waxes and oils, fatty alcohols, fatty acids, fatty acid esters, lanolin and other fatty substances.

If a preparation is formulated as an aerosol, the customary propellants, such as alkanes, fluoroalkanes and chlorofluoroalkanes, are generally used.

The cosmetic preparation may also be used to protect the hair against photochemical damage in order to prevent colour changes, bleaching or damage of a mechanical nature. In this case, a suitable formulation is in the form of a rinse-out shampoo, lotion, gel or emulsion, the preparation in question being applied before or after shampooing, before or after colouring or bleaching or before or after permanent waving. It is also possible to select a preparation in the form of a lotion or gel for styling or treating the hair, in the form of a lotion or gel for brushing or blow-waving, in the form of a hair lacquer, permanent waving composition, colorant or bleach for the hair. Besides the compounds of the formula I, the preparation having light-protection properties may comprise various adjuvants used in this type of composition, such as surfactants, thickeners, polymers, softeners, preservatives, foam stabilisers, electrolytes, organic solvents, silicone derivatives, oils, waxes, antigrease agents, dyes and/or pigments which colour the composition itself or the hair, or other ingredients usually used for hair care.

The entire disclosure of all applications, patents and publications, cited above are hereby incorporated by reference.

The compositions and their production process according to the present invention is more illustratively demonstrated but not limited by means of the following examples.

### Examples:

### Example 1

### Preparation of the Composition

### Mica/ZnO/Bariumsulfate/Ag+

50 g Shadeleaf A (Mica coated with zinc oxide and barium sulphate, Merck KGaA, Germany) are suspended in 200 ml water. 0.033 g Silver acetate are poured in 100 ml water and are dissolved using an ultrasonic bath. To the suspension of Shadeleaf A 100 ml water are added. Dissolved silver acetate is transferred into the Shadeleaf suspension after addition of 70 ml water. This is an overnight reaction at 38-39° C. (inside temperature in the round flask). Then the suspension is filtered by suction. The cake remaining on the filter is washed 3 times with 80 ml water, and then 3 times with 80 ml acetone. The product is dried during 3 days under vacuum (40° C.) in a drying oven. Final product was then sieved using a 100 micrometer sieve.

Antimicrobial efficacy was tested as following:

Testdesign “antimicrobial efficacy”:

The following aqueous dispersions are subjected to a preservation challenge test:

The challenge testing procedure consists in challenging a non-contaminated antimicrobial product according to the invention with a prescribed inoculum of suitable microorganisms and storing the inoculated product at a prescribed temperature, e.g. room temperature. The number of organisms surviving in the test products is determined at specified intervals of time.

According to the description given in the European pharmacopea (Ph.Eur.5 Ausgabe, Grundwerk 2005, §5.1.3), the challenge tests are done following the procedure given thereafter.

### Production of the Inoculum

A fresh stock culture of the specific micro-organsisms is inoculated on the surface of Agar medium B for bacteria and on the surface of Agar medium C for fungi. The bacteria culture will be incubated until sufficient sporulation (18-24 h at 30-35° C.) In order to harvest the bacteria, the surface of the Agar media is washed out with a sterile solution containing sodium chloride (9 g/l) and poured into an adequate vessel. The concentration of germs in the suspension will be adjusted with the same solution to a concentration closed to 10⁸ micro-organism/ml. Immediately after that action, a sample of this suspension is taken and the germ concentration in CFU/ml will be measured thanks to the method of membran filtration or counting on Agar plate. This value serves determining the value of the inoculum. The suspension must be used immediately.

### Method of Determination of Germ Count

In order to determine the number of micro-organisms in the inoculated preparation (containing also the antimicrobial product according to invention), the same Agar medium as for the preparation of the inoculum will be used.

The aqueous suspension/solution containing 1% of the antimicrobial product according to the invention and Example 1, or 0.5% of ZnO, or 0.1% BaSO₄ or 1% neat composition (without bound silver) is inoculated with a suspension of the tests micro-organisms (in our case Pseudomonas aeruginosa or Escherichia coli or Staphylococcus aureus or Candida albicans or Malassezia furfur or Staphylococcus epidermidis or Propionibacterium acnes or Corynebacterium xerosis) in such a way that a concentration from 10⁵ to 10⁶ microorganism/ml of the preparation is reached. The inoculated volume should not be above 1% v/v of the overall test solution. The suspension will be mixed in order to get a good homogenisation.

The inoculated preparation is stored away from the daylight at 20-25° C. In order to begin the test, 1 g or 1 ml samples from the tests preparation (containing the inoculated micro-organsisms and the antimicrobial product according to the invention) will be taken at different intervals of time (in our case , 0, 2 min, 5 min, 30 min, 1 h, 3 h, 6 h, 24 h, 48 h, 7 days, 14 days and 28 days) and the number of microorganisms will be measured using the Agar plate or the membrane filtration method. It is important to make sure that any remaining antimicrobial activity to be eliminated by dilution, filtration or specific inactivation.

### Single Compound Dispersion:

Zinc oxide (0.5 g/100 mL)

Barium sulphate (0.1 g/100 mL)

Triple Compound Dispersions:

Composition ([0.5 g Zinc oxide+0.1 g Barium sulphate+0.02 g Silver oxide]/100 mL) prepared according to example 1

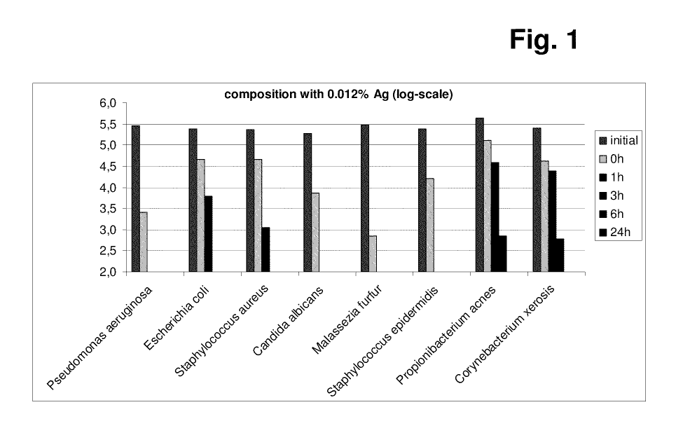

See results in Table 1, Table 2 and in FIGS. 1 to 6 for synergistic antimicrobial effects of a composition according to example 1 in comparison with a composition without bound silver ions or with single compounds as described in the figures. The advantageous antimicrobial effects are employed with such low per centages by weight of bound Ag ions, based on the composition.

TABLE 1
Challenge test results done on composition Mica/ZnO/Barium
sulphate and on composition Mica/ZnO/Barium Sulphate/Ag+ (both
samples tested at 1% suspension in water) in time intervals up to 28 d

| | 1% | 1% |
|---|---|---|
| | Mica/Bariumsulfate/ | Mica/Bariumsulfate/ZnO/Ag + |
| | ZnO in water | (0.01% as Ag₂O) in water |
| | Germs number in | Germs number in CFU/ml |
| | CFU/ml after × (time) | after × (time) |
| E. Coli | inoculum 190000 | inoculum 190000 |
| 0 | 148.000 | 102.000 |
| 24 h | 4.000 | 0 |
| 48 h | 100 | 0 |
| 7 d | 0 | 0 |
| 14 d | 0 | 0 |
| 28 d | 0 | 0 |
| P. aeruginosa | inoculum 180000 | inoculum 180000 |
| 0 | 176.000 | 111.000 |
| 24 h | 0 | 0 |
| 48 h | 0 | 0 |
| 7 d | 0 | 0 |
| 14 d | 0 | 0 |
| 28 d | 0 | 0 |
| C. albicans | inoculum 240000 | inoculum 240000 |
| 0 | 167.000 | 213.000 |
| 24 h | 130.000 | 0 |
| 48 h | 67.000 | 0 |
| 7 d | 87.000 | 0 |
| 14 d | 70.000 | 0 |
| 28 d | 28.000 | 0 |
| A. niger | inoculum 320000 | inoculum 320000 |
| 0 | 246.000 | 246.000 |
| 24 h | 321.000 | 284.000 |
| 48 h | 302.000 | 227.000 |
| 7 d | 170.000 | 151.000 |
| 14 d | 265.000 | 170.000 |
| 28 d | 302.000 | 321.000 |
| S. aureus | inoculum 230000 | inoculum 230000 |
| 0 | 148.000 | 167.000 |
| 24 h | 0 | 0 |
| 48 h | 0 | 0 |
| 7 d | 0 | 0 |
| 14 d | 0 | 0 |
| 28 d | 0 | 0 |

TABLE 2
Challenge test results done on composition Mica/ZnO/Barium
sulphate and on composition Mica/ZnO/Barium Sulphate/Ag+ (both
samples tested at 1% suspension in water) with different
microorganisms between 0 and 24 hours

| | 1% | 1% Mica/ |
|---|---|---|
| | Mica/Bariumsulphate/ | Bariumsulphate/ZnO/Ag + |
| | ZnO in water | (0.01% as Ag₂O) in water |
| | Germs number in | Germs number in |
| | CFU/ml after × (time) | CFU/ml after × (time) |
| C. xerosis | inoculum 240000 | inoculum 220000 |
| 0 | 259.000 | 102.000 |
| 2 min | 269.000 | 34.000 |
| 5 min | 185.000 | 21.000 |
| 30 min | 185.000 | 21.000 |
| 1 h | 176.000 | 11.000 |
| 6 h | 167.000 | 200 |
| 24 h | 100 | 0 |
| S. epidermidis | inoculum 230000 | inoculum 250000 |
| 0 | 248.000 | 148.000 |
| 2 min | 248.000 | 13.000 |
| 5 min | 222.000 | 6.800 |
| 30 min | 222.000 | 100 |
| 1 h | 194.000 | 100 |
| 6 h | 42.000 | 0 |
| 24 h | 100 | 0 |
| P. acnes | inoculum 250000 | inoculum 420000 |
| 0 | 333.000 | 287.000 |
| 6 h | 100 | 100 |
| 24 h | 0 | 0 |

FIG. 1 describes challenge test results according to the challenge test as described above done on composition ZnO [0.5%]/BaSO₄ [0.4%]+bound Ag (composition of example 1) tested at 1% suspension in water with different microorganism. The results that means the germs numbers are given in log scale.

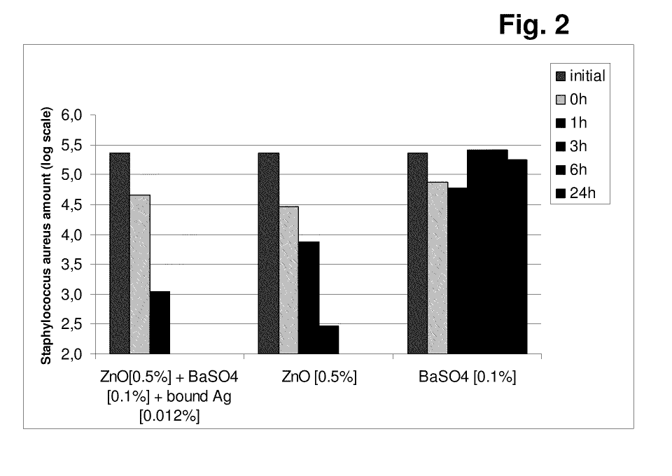

FIG. 2 describes challenge tests results in log scale done on pure BaSO₄, pure ZnO and on ZnO [0.5%]/BaSO₄ [0.4%]+bound Ag (composition of example 1), testing the synergistic antimicrobial effects on S. aureus. Results in log scale.

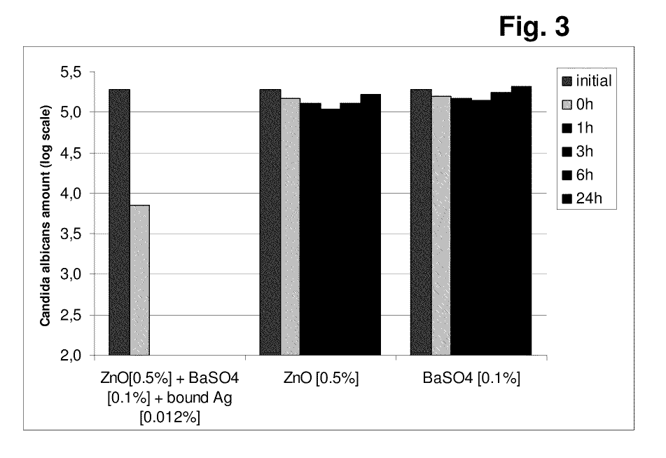

FIG. 3 describes challenge tests results in log scale done on pure BaSO₄, pure ZnO and on ZnO [0.5%]/BaSO₄ [0.4%]+bound Ag ion (composition of example 1), testing the synergistic antimicrobial effects on C. albicans. Results in log scale.

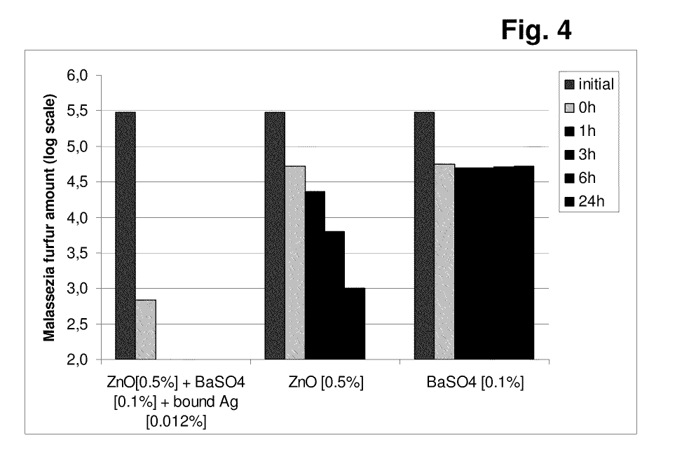

FIG. 4 describes challenge tests results in log scale done on pure BaSO₄, pure ZnO and on ZnO [0.5%]/BaSO₄ [0.4%]+bound Ag ion (composition of example 1), testing the synergistic antimicrobial effects on M. furfur. Results in log scale.

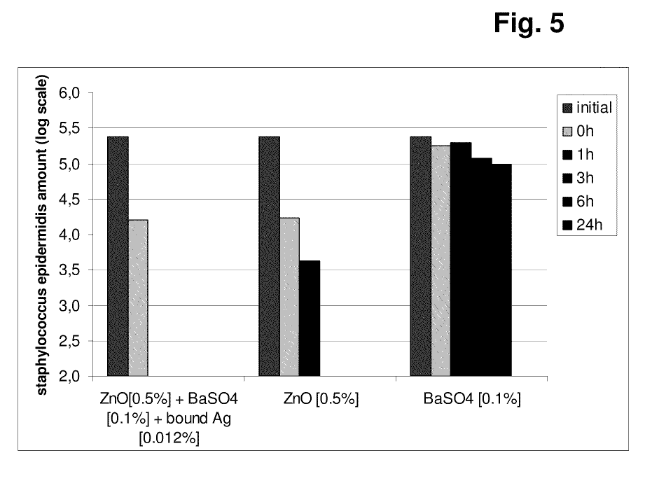

FIG. 5 describes challenge tests results in log scale done on pure BaSO₄, pure ZnO and on ZnO [0.5%]/BaSO₄ [0.4%]+bound Ag ion (composition of example 1), testing the synergistic antimicrobial effects on S. epidermidis. Results in log scale.

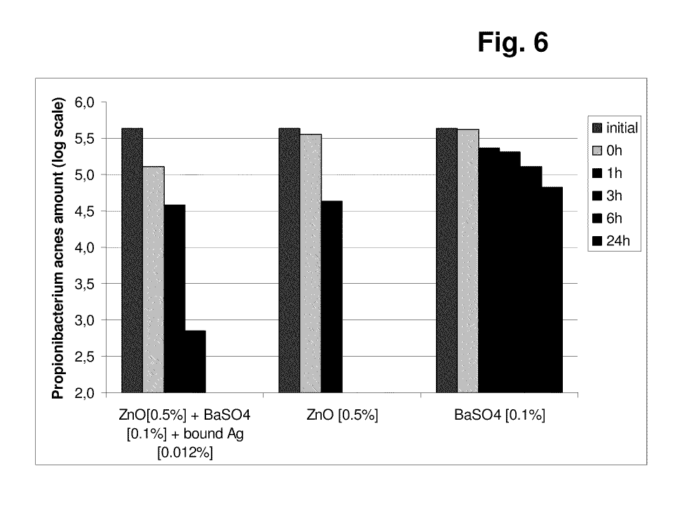

FIG. 6 describes challenge tests results in log scale done on pure BaSO₄, pure ZnO and on ZnO [0.5%]/BaSO₄ [0.4%]+bound Ag ion (composition of example 1), testing the synergistic antimicrobial effects on P. acnes. Results in log scale.

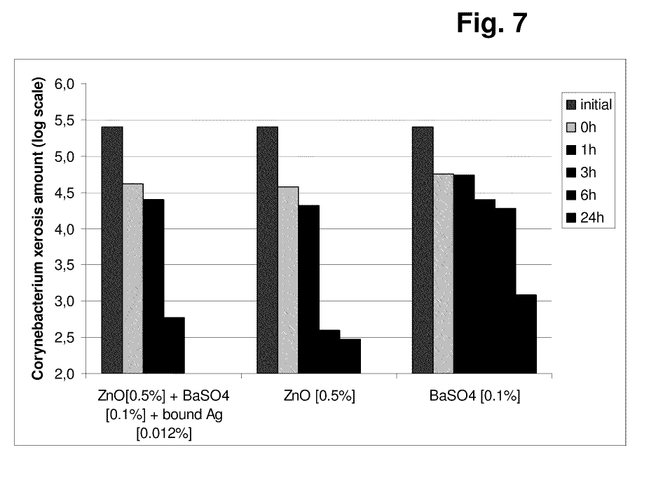

FIG. 7 describes challenge tests results in log scale done on pure BaSO₄, pure ZnO and on ZnO [0.5%]/BaSO₄ [0.4%]+bound Ag ion (composition of example 1), testing the synergistic antimicrobial effects on Cornebacterium xerosis. Results in log scale.

### APPLICATION EXAMPLES

A) Roll-On Formulation

| Ingredient | INCI | Weight-% |
|---|---|---|
| Polyethylenglycol | PEG-8 | 10 |
| 400 | | |
| Ethanol | Alcohol | 10 |
| Phenonip | Phenoxyethanol, Methylparaben, | 0.5 |
| | Butylparaben, Ethylparaben, | |
| | Propylparaben | |
| Natrosol HHR 250 | Hydroxyethylcellulose | 0.8 |
| Composition of | | 1 |
| example 1 | | |
| Water | Aqua | ad 100 |

B) Deodorant Formulation

| Ingredient | INCI | Weight-% |
|---|---|---|
| Paraffinum | Paraffinum Liquidum | 2 |
| Liquidum | | |
| Arlamol HD | Isohexadecane | 2 |
| IPP | Isopropyl Palmitate | 3 |
| Soy been oil | Glycine Soja (Soybean Oil) | 0.5 |
| Mirasil DM 350 | Dimethicone | 1 |
| Lanette O | Cetearyl Alcohol | 1 |
| Span 60 | Sorbitan Stearate | 1.5 |
| Montanov 68 | Cetearyl Alcohol, | 4 |
| | Cetearyl Glucoside | |
| Water | Aqua | ad 100 |
| Glycerol 87% | Glycerin | 5 |
| Rhodicare S | Xanthan Gum | 0.3 |
| Germaben II | Propylene Glycol, Diazolidinyl | 1 |
| | Urea, Methylparaben, | |
| | Propylparaben | |
| Composition of | | 1 |
| example 1 | | |
| Citric acid | Citric Acid | q.s. |

The above formulation A or B may of course contain one or more actives among the following list: Triethyl Citrate, Aluminium Chlorohydrate, Ethylhexylglycerol, Farnesol, Polyaminopropyl Biguanide, Aluminium Circonium ,Tetrachlorohydrex GLY, Pentetic Acid, Diisopropylamine Aminoethylpropanol, Zinc Ricinoleate, aluminium salts, Lactic Acid, Triclosan.

C) Waterproof Suncare Spray [Amounts are Given in % by Weight of the Formulation]

| A | | | |
|---|---|---|---|
| composition of example 1 | 1 | 1 | 2 |
| Diethylhexyl | | 0.5 | |
| Syringylidenemalonate, | | | |
| Caprylic/Capric | | | |
| Triglyceride | | | |
| (Oxynex ® ST Liquid) | | | |
| RonaCare ® AP | | 2 | |
| Ascorbyl Palmitate | | | 1 |
| Caprylic/capric Triglyceride | 7 | 7 | 7 |
| (Miglyol 812 N) | | | |
| Butylphthalimide | 10 | 10 | 10 |
| isopropylphthalimide | | | |
| (Pelemol ® BIP) | | | |
| C12-15 alkyl benzoate | 10 | 10 | 10 |
| (Tegosoft ® TN) | | | |
| Phenethyl benzoate | 5 | 5 | 5 |
| (X-Tend 226) | | | |
| RonaCare ® | 1 | 1 | 1 |
| Tocopherolacetat | | | |
| B | | | |
| Cyclopentasiloxane | 43.8 | 41.3 | 41.8 |
| (Dow Corning 245) | | | |
| Phenyltrimethicone | 2 | 2 | 2 |
| (Dow Corning 556) | | | |
| Cyclopentasiloxane, | 20 | 20 | 20 |
| dimethiconol | | | |
| Dow Corning 1501 Fluid | | | |
| Parfum (q.s.) | 0.2 | 0.2 | 0.2 |

Procedure: phase A will be mixed at room temperature. Phase B will be mixed at room temperature and will be given to phase A while stirring.

The above formulation may of course contain one or more actives among the following list: Myrtrimonium Bromid, Lactic Acid, Chlorohexidine Digluconate, Salicylic Acid, Phenoxyisopropanol, Isopropanol, Farnesol, Glycolic Acid, Tannic acid, Sulfur, Alcohol, Triclosan, Zinc Gluconate, Zinc PCA, Camphor, Aluminium salts, Sodium Lactate, Polyaminopropyl Biguanide, Zinc Acetat. Zinc Pyrithion, Piroctone Olamine, Ketocokanzole, Tropolone, Hinokitol, Selenium Sulfide, Climbazole, Sodium Salicylate, Ciclopiroxolamine, Neem, Basilic oil, Ichtammol, Melaleuca Alternifolia, Centaurea Cyanus, Melia Azadirachta, Sulfur, Clotrimazole, Crotamiton, Zinc Salicylate, Selenium Sulfide, Tussilago farfara, Arctium lappa, Piroctone Olamine, Salicylic Acid, Zinc Sulfate, Rosmarinus officinalis, Ketoconazole.

D) Pump Hairspray [Amounts are Given in % by Weight of the Formulation]:

| | | | |
|---|---|---|---|
| A | | | |
| composition of example 1 | 1 | 2 | 4 |
| Ethanol 96% pure | Ad 100 | Ad 100 | Ad 100 |
| PVP/VA copolymer | 6 | 6 | 6 |
| PVP/VA W 735 | | | |
| B | | | |
| Diethylhexyl | 0.06 | 0.25 | 0.5 |
| Syringylidenemalonate, | | | |
| Caprylic/Capric Triglyceride | | | |
| (Oxynex ® ST Liquid) | | | |
| PEG-75 Lanolin | 0.2 | 0.2 | 0.2 |
| BHT | | | |
| (Solan E-Low Dioxane) | | | |
| Parfum | 0.1 | 0.1 | 0.1 |
| (Frag 280853 Green Activating) | | | |
| C | | | |
| Aqua (Water) | 13 | 13 | 13 |
| Titriplex III (sodium EDTA) | 0.1 | 0.1 | 0.1 |
| PEG-12 dimethicone | 0.5 | 0.5 | 0.5 |
| Dow Corning 193 Fluid | | | |
| 0.1% D&C Red No 33 (CI 17200) | 0.2 | 0.2 | 0.2 |
| in water | | | |
| PEG-40 Hydrogenated Castor Oil | 1 | 1 | 1 |
| (Cremophor RH 410) | | | |

Procedure: Phase B is given to phase A while stirring. Phase C is mixed and added to the combined phases A and B. Stirring until homogeneity.

The above formulation may of course contain one or more actives among the following list:

Zinc Pyrithione, Piroctone Olamine, Ketoconazole, Tropolone, Hinokitol Selenium Sulfide, Salicylic Acid, Climbazole, Sodium Salicylate, Ciclopiroxolamine, Neem, Basilic oil, Ichtammol, Melaleuca Alternifolia, Centaurea Cyanus, Melia Azadirachta, Farnesol, Sulfur, Clotrimazole, Crotamiton, Zinc Salicylate, Tussilago farfara, Arctium lappa, Zinc Sulfate, Rosmarinus officinalis, Ketoconazole.

E) W/O Emulsions [Amounts are Given in % by Weight of the Formulation]

| Emulsion | A | B | C | D | E | F |
|---|---|---|---|---|---|---|
| Polyglyceryl-2- | 3 | 5 | 3 | | | |
| Dipolyhydroxystearate | | | | | | |
| PEG-30 | | | 2 | 3 | 4 | 5 |
| Dipolyhydroxystearate | | | | | | |
| Sodium starch | 0.5 | 0.4 | | 0.3 | | 1 |
| Octenylsuccinate | | | | | | |
| Glycine | 0.3 | 0.3 | 0.5 | 0.4 | | |
| Alcohol | | 5 | 2 | 5 | 4 | |
| Magnesium sulfate | 0.2 | 0.3 | 0.3 | 0.4 | 0.5 | 0.2 |
| C₁₂₋₁₅ Alkyl Benzoate | 5 | 3 | | | 5 | |
| C₁₂₋₁₃ Alkyl Tartrate | | 2 | | | | |
| Butylene glycol Dicaprylate/ | 5 | | | | 3 | 3 |
| Dicaprate | | | | | | |
| Dicaprylyl Ether | | | | | 2 | |
| Mineral oil | | 4 | | 6 | | 8 |
| Octyldodecanol | 2 | | | | | |
| Dicaprylcaprate | | 2 | | | 2 | 2 |
| Cyclomethicone | 5 | | 5 | 10 | | |
| Dimethicone | | | | 5 | | |
| Isohexadecane | | 1 | | | | |
| Butylene glycol | 5 | 8 | | | | 3 |
| Propylene glycol | | | 1 | | 5 | 3 |
| Glycerol | 3 | 5 | 7 | 10 | 3 | 3 |
| C18-38 Acid triglyceride | 0.5 | | 1 | | 1 | |
| Titanium dioxide | 5 | 6 | 4 | | | 4 |
| Zinc oxide | 5 | | | | | |
| Bis-Ethylhexyloxyphenol | | 3 | 3 | 2 | | |
| Methoxyphenyltriazin | | | | | | |
| Ethylhexyl triazone | | 4.5 | 3 | | 3 | |
| composition of example 1 | 2.0 | 0.5 | 1.0 | 1.0 | 3.0 | 1.5 |
| Diethylhexyl | | | 1.5 | 4 | | |
| butamidotriazone | | | | | | |
| Butyl | 2 | 3 | 4 | | 1 | 3 |
| Methoxydibenzoylmethane | | | | | | |
| Uvinul ® A Plus | | | | 4 | 2 | |
| Ethylhexyl | | | | | 7 | 5 |
| methoxycinnamate | | | | | | |
| Benzotriazole coppled to | 4 | | 6 | | | |
| gelatine | | | | | | |
| Taurine | 0.1 | | | 0.5 | 0.2 | |
| Vitamin E Acetate | 0.2 | 02 | | 0.3 | 0.1 | 0.5 |
| Na₂H₂EDTA | 0.1 | 0.1 | 0.2 | 0.2 | 0.2 | 0.5 |
| C8-C16 Alkylpolyglycoside | 1 | | | | | |
| Parfum, preservative | q.s. | q.s | q.s | q.s | qs. | qs. |
| Dye. | q.s. | q.s. | q.s. | q.s | q.s. | q.s. |
| Sodium hydroxid | q.s. | q.s. | q.s. | q.s | q.s. | q.s. |
| Water | ad 100 | ad 100 | ad 100 | ad 100 | ad 100 | ad 100 |

The above formulation may of course contain one or more actives among the following list:

Zinc Pyrithione, Piroctone Olamine, Ketoconazole, Tropolone, Hinokitol, Selenium Sulfide, Salicylic Acid, Climbazole, Sodium Salicylate, Ciclopiroxolamine, Neem, Basilic oil, Ichtammol, Melaleuca Alternifolia, Centaurea Gyanus, Melia Azadirachta, Farnesol, Sulfur , Clotrimazole, Zinc Salicylate, Tussilago farfara, Arctium lappa, Zinc Sulfate, Rosmarinus officinalis, Myrtrimonium Bromid, Lactic Acid, Chlorohexidine Digluconate, Phenoxyisopropanol, Isopropanol, Farnesol, Glycolic Acid, Tannic acid, Alcohol, Triclosan, Zinc Gluconate, Zinc PCA, Camphor, Aluminium salts, Sodium Lactate, Polyaminopropyl Biguanide, Zinc Acetat, Triethyl Citrate, Ethylhexylglycerol, Aluminium Circonium, Tetrachlorohydrex GLY, Pentetic Acid, Diisopropylamine Aminoethylpropanol, Zinc Ricinoleate, Aluminium Sesquichlorohydrate, Lactic Acid, Triclosan.

F) Hair Care Formulation [Amounts are Given in % by Weight of the Formulation)

| Ingredient | A | B | C | D | E | F |
|---|---|---|---|---|---|---|
| Disodium EDTA | 0.1 | 0.1 | 0.1 | 0.1 | 0.1 | 0.1 |
| Oxynex ® ST | 2 | 2 | 2 | 2 | 2 | 2 |
| composition of example 1 | 0.1 | 0.25 | 0.5 | 1.5 | 2 | 4 |
| Hexamidine diisethionate | 0.1 | 0 | 0 | 0 | 0 | 0 |
| Tetrahydrocurcumin | 0 | 0.5 | 0 | 0 | 0 | 0 |
| Glycyrrhetinic acid | 0 | 0 | 0.3 | 0 | 0 | 0 |
| Thiotaine ® | 0 | 0 | 0 | 5 | 0 | 0 |
| N-undecylenoyl-L-phenyl- | 0 | 0 | 0 | 0 | 1 | 0 |
| alanine | | | | | | |
| N-acetyl glucosamine | 0 | 0 | 0 | 0 | 0 | 2 |
| Niacinamide | 5 | 5 | 5 | 5 | 5 | 5 |
| Citric acid | 0.015 | 0 | 0 | 0 | 0 | 0 |
| Isohexadecane | 3 | 3 | 3 | 3 | 3 | 3 |
| Isopropyl isostearate | 1.33 | 1.33 | 1.33 | 1.33 | 1.33 | 1.33 |
| Isopropyl N-laurosyl- | 0 | 0 | 5 | 0 | 0 | 0 |
| sarcosinate | | | | | | |
| Sucrose polycottonseedate | 0.67 | 0.67 | 0.67 | 0.67 | 0.67 | 0.67 |
| Polymethylsilsesquioxane | 0.25 | 0.25 | 0.25 | 0.25 | 0.25 | 0.25 |
| Cetearyl glucoside + cetearyl | 0.2 | 0.2 | 0.2 | 0.2 | 0.2 | 0.2 |
| alcohol | | | | | | |
| Behenyl alcohol | 0.4 | 0.4 | 0.4 | 0.4 | 0.4 | 0.4 |
| Ethylparaben | 0.2 | 0.2 | 0.2 | 0.2 | 0.2 | 0.2 |
| Propylparaben | 0.1 | 0.1 | 0.1 | 0.1 | 0.1 | 0.1 |
| Cetyl alcohol | 0.32 | 0.32 | 0.32 | 0.32 | 0.32 | 0.32 |
| Stearyl alcohol | 0.48 | 0.48 | 0.48 | 0.48 | 0.48 | 0.48 |
| Tocopheryl acetate | 0.5 | 0.5 | 0.5 | 0.5 | 0.5 | 0.5 |
| PEG-100 stearate | 0.1 | 0.1 | 0.1 | 0.1 | 0.1 | 0.1 |
| Glycerol | 7 | 7 | 7 | 7 | 7 | 7 |
| Titanium dioxide | 0.6 | 0.6 | 0.6 | 0.6 | 0.6 | 0.6 |
| Polyacrylamide + C13-14 | 3 | 2 | 2 | 2 | 2 | 2 |
| isoparaffin + laureth-7 | | | | | | |
| Panthenol | 1 | 1 | 1 | 1 | 1 | 1 |
| Benzyl alcohol | 0.4 | 0.4 | 0.4 | 0.4 | 0.4 | 0.4 |
| Dimethicone + dimethiconol | 2 | 2 | 2 | 2 | 2 | 2 |
| Water | to 100 | to 100 | to 100 | to 100 | to 100 | to 100 |

Continuation: Hair care formulation [amounts are given in % by weight of the formulation]

| ingredient | G | H | I |
|---|---|---|---|
| Disodium EDTA | 0.1 | 0.1 | 0.1 |
| Oxynex ® ST | 2 | 2 | 2 |
| composition of example 1 | 0.5 | 3.5 | 1.5 |
| Cetyl pyridinium chloride | 0.2 | 0 | 0 |
| Pitera ® | 0 | 10 | 0 |
| Ascorbyl glycoside | 0 | 0 | 2 |
| Niacinamide | 5 | 5 | 5 |
| Polyquaternium 37 | 0 | 0 | 0 |
| Isohexadecane | 3 | 3 | 3 |
| Isopropyl isostearate | 1.3 | 1.3 | 1.3 |
| Sucrose polycottonseedate | 0.7 | 0.7 | 0.7 |
| Polymethylsilsesquioxane | 0.25 | 0.25 | 0.25 |
| Cetearyl glucoside + cetearyl alcohol | 0.2 | 0.2 | 0.2 |
| Behenyl alcohol | 0.4 | 0.4 | 0.4 |
| Ethylparaben | 0.2 | 0.2 | 0.2 |
| Propylparaben | 0.1 | 0.1 | 0.1 |
| Cetyl alcohol | 0.3 | 0.3 | 0.3 |
| Stearyl alcohol | 0.5 | 0.5 | 0.5 |
| Tocopheryl acetate | 0.5 | 0.5 | 0.5 |
| PEG-100 stearate | 0.1 | 0.1 | 0.1 |
| Glycerol | 7 | 7 | 7 |
| Titanium dioxide | 0.6 | 0.6 | 0.6 |
| Polyacrylamide + C13-14 isoparaffin + | 2 | 2 | 2 |
| laureth-7 | | | |
| Panthenol | 1 | 1 | 1 |
| Benzyl alcohol | 0.4 | 0.4 | 0.4 |
| Dimethicone + dimethiconol | 2 | 2 | 2 |
| Water (to 100 g) | to 100 | to 100 | to 100 |

The above formulation may of course contain one or more actives among the following list:

Zinc Pyrithione, Piroctone Olamine, Ketoconazole, Tropolone, Hinokitol, Selenium Sulfide, Salicylic Acid, Climbazole, Sodium Salicylate, Ciclopiroxolamine, Neem, Basilic oil, Ichtammol, Melaleuca Alternifolia, Centaurea Cyanus, Melia Azadirachta, Farnesol, Sulfur, Clotrimazole, Crotamiton, Zinc Salicylate, Tussilago farfara, Arctium lappa, Zinc Sulfate, Rosmarinus officinalis.

G) O/W Emulsion [Amounts are Given in % by Weight of the Formulation]

| Emulsion | A | B | C | D | E | F |
|---|---|---|---|---|---|---|
| Glyceryl Stearate Citrate | 2.5 | 2 | 3 | | | |
| Sorbitan stearate | 0.5 | | | 2 | 1.5 | 2 |
| Polyglyceryl-3 | | | | 2.5 | 3 | 3 |
| Methylglycose Distearate | | | | | | |
| Polyglyceryl-2 | | 0.8 | | | | 0.5 |
| Dipolyhydroxystearate | | | | | | |
| Cetearyl alcohol | | | | 1 | | |
| Stearyl alcohol | 2 | | | | | 2 |
| Cetyl alcohol | | 1 | | | 3 | |
| Acrylates/C₁₀₋₃₀ Alkyl | | 0.2 | | | 0.1 | |
| Acrylat Crosspolymer | | | | | | |
| Carbomer | | 0.2 | 0.3 | 0.2 | | |
| Xanthan Gum | 0.4 | | 0.2 | 0.2 | 0.3 | 0.4 |
| C₁₂₋₁₅ Alkyl Benzoat | 5 | 3 | | | 5 | |
| C₁₂₋₁₃ Alkyl Tartrate | | 2 | | | | |
| Butylenglycol Dicaprylate/ | 5 | | | | 3 | 3 |
| Dicaprate | | | | | | |
| Dicaprylyl Ether | | | | | 2 | |
| Octyldodecanol | 2 | | | | | |
| Dicaprylcaprate | | 2 | | | 2 | 2 |
| Cyclomethicone | 5 | | 5 | 10 | | |
| Dimethicone | | | | 5 | | |
| Isohexadecane | | 1 | | | | |
| Butylene glycol | 5 | 8 | | | | 3 |
| Propylene gycol | | | 1 | | 5 | 3 |
| Glycerol | 3 | 5 | 7 | 10 | 3 | 3 |
| C18-C38 Acid triglyceride | 0.5 | | 1 | | 1 | |
| Titanium dioxide | 5 | | | 2 | | |
| 2,2′-Methylen-bis-(6- | 2.5 | | | | | |
| (2H-benzotriazol-2-yl)-(1,1,3,3- | | | | | | |
| tetramethylbutyl)phenol) | | | | | | |
| 2,4,6-Tris-(biphenyl)- | | 2 | | | | |
| 1,3,5-triazin | | | | | | |
| C8-C16 Alkylpolyglycosid | 1 | 0.6 | | | | |
| UVASorb ® K2A | | | 2 | | | |
| Uvinul ® A Plus | 2 | | | | | 1 |
| Homosalate | | 5 | | 1 | | |
| Phenylbenzimidazole | | | 2 | | | 1 |
| sulfonic acid | | | | | | |
| Benzophenone-3 | 2 | | | | 2 | |
| Octylsalicylate | 5 | 5 | | 2 | | |
| Octocrylene | 2 | | | | 3 | 1 |
| composition of example 1 | 1 | 2 | 3 | 1 | 2 | 3 |
| Bis-Ethylhexyloxyphenol | | 3 | 2 | 1 | | |
| Methoxyphenyltriazin | | | | | | |
| Parsol ® SLX | | | 3 | | | |
| Dihydroxyacetate | | | | | 4 | |
| Taurine | 0.1 | | | 0.5 | 0.2 | |
| 8-Hexadecen-1,16- | | 0.2 | | | | |
| dicarbon acid | | | | | | |
| Vitamin E Acetate | 0.2 | 0.2 | | 0.3 | 0.1 | 0.5 |
| Na₂H₂EDTA | 0.1 | 0.1 | 0.2 | 0.2 | 0.2 | 0.5 |
| Parfum, preservative | q.s. | q.s. | q.s. | q.s. | q.s. | q.s. |
| Dye. | q.s. | q.s. | q.s. | q.s. | q.s. | q.s. |
| Sodium hydroxide | q.s. | q.s. | q.s. | q.s. | q.s. | q.s. |
| Water | ad 100 | ad 100 | ad 100 | ad 100 | ad 100 | ad 100 |

Continuation OM-Emulsions [amounts are given in % by weight of the formulation]

| Emulsion | G | H | I | K | L | M |
|---|---|---|---|---|---|---|
| Ceteareth-20 | 1 | 1.5 | 1 | | | |
| Sorbitan stearate | | | 0.5 | | 0.5 | |
| Glyceryl Stearate SE | | | | 1 | 1 | 1.5 |
| Emulgade F ® | | | | 2.5 | 2.5 | 3 |
| Cetearyl alcohol | | | | 1 | | |
| Stearyl alcohol | | | | | 1.5 | |
| Cetyl alcohol | | | 0.5 | | | 2 |
| Acrylates/C₁₀₋₃₀ Alkyl | 0.2 | 0.4 | 0.3 | 0.1 | | |
| Acrylate Crosspolymer | | | | | | |
| Carbomer | | | | | 0.3 | |
| Xanthan Gum | | | | 0.4 | | 0.4 |
| C₁₂₋₁₅ Alkyl Benzoate | 5 | 3 | | | 5 | |
| 2-Phenylbenzoate | | 2 | | | | |
| Butylene glycol | 5 | | | | 3 | 2 |
| Dicaprylate/Dicaprate | | | | | | |
| Dicaprylyl Ether | | | | | 2 | |
| Diethylhexylnaphthalate | 2 | | | | | |
| Dicaprylcaprate | | 2 | | | 2 | 2 |
| Cyclomethicone | 5 | | 5 | 10 | | |
| Isohexadecane | | | | 5 | | |
| Mineral oil | | 1 | | | | |
| Propylene glycol | | | 4 | | | |
| Glycerol | 5 | 7 | 3 | 5 | 6 | 8 |
| C18-38 Acid triglyceride | 0.5 | | 1 | | 1 | |
| Titanium dioxide | 5 | | 3 | 2 | | |
| NeoHeliopan ® AP | | 2 | | | 1 | 1 |
| Phenylbenzimidazol | 1 | | | 1 | 2 | 1 |
| Sulfonic acid | | | | | | |
| Ethylhexylmethoxycinnamate | 5 | | 4 | 4 | | |
| Ethylhexyltriazon | | 2 | | 1 | | |
| Diethylhexyl | 1 | | | | | |
| butamidotriazan | | | | | | |
| Butyl Methoxydibenzoylmethane | 2.5 | | 2 | 2 | | 1 |
| Bis-Ethylhexyloxyphenol | 2 | | | | | |
| Methoxyphenyltriazin | | | | | | |
| 4-Methylbenzyliden | 3 | | | | | |
| Camphor | | | | | | |
| Parsol ® SLX | | | | | 2 | |
| composition of example 1 | 1 | 2 | 4 | 0.5 | 1.5 | 3 |
| Creatinine | 0.1 | 0.01 | 0.05 | | | |
| Creatin | 0.5 | 0.2 | 0.1 | | | |
| Licorice | | | | 0.5 | | |
| Extract/Licochalcon | | | | | | |
| Vitamin E Acetat | 0.2 | | | 0.5 | 0.5 | 0.5 |
| Tapioka Starch | | 3 | | | 2 | |
| Na₂H₂EDTA | 0.1 | | 0.2 | | | 0.5 |
| Parfum, preservative | q.s. | q.s. | q.s. | q.s. | q.s. | q.s. |
| Dye | q.s. | q.s. | q.s. | q.s. | q.s. | q.s. |
| Sodium hydroxide | q.s. | q.s. | q.s. | q.s. | q.s. | q.s. |
| Water | ad 100 | ad 100 | ad 100 | ad 100 | ad 100 | ad 100 |

Continuation O/W Emulsions [amounts are given in % by weight of the formulation]

| Emulsion | N | O | P | Q | R | S |
|---|---|---|---|---|---|---|
| Glycerylstearate SE | | 2 | | 2 | | |
| Glycerylstearate | 2 | | 2 | | | |
| PEG-40 Stearate | | | 2 | | 1 | |
| PEG-10 Stearate | | | | 2.5 | 1 | |
| Ceteareth-20 | | | | | | 2.6 |
| Natrium Cetyl Phosphate | | | | | 2 | |
| Glyceryl Stearate, | | | | | | 5.4 |
| Ceteareth-12, Ceteareth- | | | | | | |
| 20, Cetearyl Alcohol, Cetyl | | | | | | |
| Palmitate | | | | | | |
| Stearic acid | 3 | 2 | | | 2 | |
| Stearyl alcohol | | 2 | 2 | | | |
| Stearyl alcohol | 0.5 | | 2 | | | |
| Cetyl alcohol | 3 | | | 2 | | |
| Acrylates/C₁₀₋₃₀ Alkyl | | | 0.2 | | 0.4 | |
| Acrylate Crosspolymer | | | | | | |
| Carbomer | | 0.3 | | 0.3 | 0.3 | |
| Xanthan Gum | | 0.3 | 0.4 | | | |
| C₁₂₋₁₅ Alkyl Benzoate | 5 | | | | 5 | 3 |
| 2-Phenylbenzoate | 5 | | | | | |
| Butylene glycol | | 5 | | 4 | | 3 |
| Dicaprylate/Dicaprate | | | | | | |
| Dicaprylyl Ether | | 2 | | | 3 | |
| Diethylhexylnaphthalate | 3 | | | | | |
| Cyclomethicone | 2 | | 10 | 2 | | |
| Isohexadecane | | | | 2 | 3 | |
| Mineral oil | | | | | 3 | |
| Propandiol | | 3 | | 5 | | |
| Glycerol | 3 | 5 | 10 | 7 | 4 | 5 |
| Titanium dioxide | 2 | 4 | | | | |
| Zinc oxide | | | | | 2 | |
| Drometrizole Trisiloxane | | | | | 3 | |
| Ethylhexylmethoxycinnamate | | 6 | 5 | | | |
| Phenylbenzimidazol | | 0.5 | 2 | | 1 | |
| Sulfonic acid | | | | | | |
| Homosalate | 5 | | | 7 | | |
| Butyl Methoxydibenzoylmethane | | 3 | | | | |
| Bis-Ethylhexyloxyphenol | | 2 | 3 | | | |
| Methoxyphenyltriazin | | | | | | |
| Octylsalicylate | | | | 5 | | |
| Octocrylene | | | | | 3 | |
| composition of example 1 | 0.25 | 1.5 | 0.5 | 2.5 | 1 | 5 |
| Parsol ® SLX | 4 | | | | | 5 |
| PVP Hexadecen | 0.5 | | 1 | | 0.8 | |
| Copolymer | | | | | | |
| Coenzym Q 10 | 0.2 | 0.02 | | 0.3 | | |
| Vitamin E Acetate | 0.2 | | 0.3 | | 0.8 | 0.5 |
| Na₂H₂EDTA | 0.1 | | | | | 0.5 |
| Parfum, preservative | q.s. | q.s. | q.s. | q.s. | q.s. | q.s. |
| Dye | q.s. | q.s. | q.s. | q.s. | q.s. | q.s. |
| Sodium hydroxide | q.s. | q.s. | q.s. | q.s. | q.s. | q.s. |
| Water | ad 100 | ad 100 | ad 100 | ad 100 | ad 100 | ad 100 |

The above formulation may of course contain one or more actives among the following list: Zinc Pyrithione, Piroctone Olamine, Ketoconazole, Tropolone, Hinokitol, Selenium Sulfide, Salicylic Acid, Climbazole, Sodium Salicylate, Ciclopiroxolamine, Neem, Basilic oil, Ichtammol, Melaleuca Alternifolia, Centaurea Cyanus, Melia Azadirachta, Farnesol, Sulfur, Clotrimazole, Crotamiton, Zinc Salicylate, Tussilago farfara, Arctium lappa, Zinc Sulfate, Rosmarinus officinalis, Myrtrimonium Bromid, Lactic Acid, Chlorohexidine Digluconate, Phenoxyisopropanol, Isopropanol, Farnesol, Glycolic Acid, Tannic acid, Alcohol, Triclosan, Zinc Gluconate, Zinc PCA, Camphor, Aluminium salts, Sodium Lactate, Polyaminopropyl Biguanide, Zinc Acetate, Triethyl Citrate, Ethylhexylglycerol, Aluminium Circonium, Tetrachlorohydrex GLY, Pentetic Acid, Diisopropylamine Aminoethylpropanol, Zinc Ricinoleate, Aluminium Sesquichlorohydrate, Lactic Acid, Triclosan.

H) Hydrodisperisions (Lotions and Sprays) [Amounts are Given in % by Weight of the Formulation]

| | A | B | C | D | E | F |
|---|---|---|---|---|---|---|
| Glyceryl Stearate Citrate | | 0.4 | | | | |
| Cetyl Alcohol | | | | | 2 | |
| Natrium Carbomer | | | | | 0.3 | |
| Acrylates/C₁₀₋₃₀ Alkyl | 0.3 | | 0.3 | 0.4 | 0.1 | 0.1 |
| Acrylate Crosspolymer | | | | | | |
| Ceteareth-20 | | | 1 | | | |
| Xanthan Gum | | | | 0.15 | | 0.5 |
| Dimethicone/Vinyl | | | | 5 | | 3 |
| Dimethicone | | | | | | |
| Crosspolymer | | | | | | |
| UVASorb ® K2A | | | | | 3.5 | |
| Uvinul ® A Plus | 0.25 | | | 0.5 | 2 | 1.5 |
| Butyl Methoxydibenzoylmethane | 1.2 | | 3.5 | | | |
| Bis-Ethylhexyloxyphenol | 2 | 2 | | 0.25 | | |
| Methoxyphenyl Triazin | | | | | | |
| Terephthalidene | | | | | | 0.5 |
| Dicampher Sulfonic acid | | | | | | |
| Dinatrium Phenyl | | | | | | 1 |
| Dibenzimidazol | | | | | | |
| Tetrasulfonate | | | | | | |
| Phenylbenzimidazol | | | 2 | | | |
| Sulfonic acid | | | | | | |
| Ethylhexyl | 5 | | 7 | | 5 | 8 |
| Methoxycinnamate | | | | | | |
| Diethylhexyl Butamido | | | 2 | 2 | | |
| Triazon | | | | | | |
| Ethylhexyl Triazon | 4 | 3 | | | 4 | |
| Octocrylene | | | | 10 | | 2.5 |
| Composition of example 1 | 0.25 | 1.5 | 0.5 | 2.5 | 1 | 5 |
| C₁₂₋₁₅ Alkyl Benzoate | 2 | | 2.5 | | | |
| Phenethyl Benzoate | 4 | | | 7.5 | | 5 |
| C₁₈₋₃₆ Triglycerid Fatty | | | 1 | | | |
| acid | | | | | | |
| Butylene glycol | | | | | 6 | |
| Dicaprylate/Dicaprate | | | | | | |
| Dicaprylyl Carbonate | | 3 | | | | |
| Dicaprylylether | | 2 | | | | |
| Cyclomethicone | | | | 1.5 | | |
| Lanoline | | | | | 0.35 | |
| PVP Hexadecen | 0.5 | | 0.5 | | 0.5 | 1 |
| Copolymer | | | | | | |
| Ethylhexyloxyglycerol | | 0.75 | | 1 | | 0.5 |
| Glycerol | 10 | 5 | 5 | | 5 | 15 |
| Butylene glycol | | 7 | | | | |
| Glycin Soja | | | | 1 | | |
| Vitamin E Acetate | 0.5 | 0.25 | 0 5 | 0.25 | 0.75 | 1 |
| α-Glycosylrutin | | | | | 0.25 | |
| Trinatrium EDTA | | 1 | 1 | 0.1 | 0.2 | |
| Idopropinylbutylcarbamate | 0.2 | 0.1 | | | | 0.15 |
| Methylparabene | 0.5 | | 0.2 | | 0.15 | |
| Phenoxyethanol | 0.5 | 0.4 | 0.4 | | 1 | 0.6 |
| Ethanol | 3 | 10 | 4 | 3.5 | | 1 |
| Parfum, Dye | q.s. | q.s. | q.s. | qs. | q.s. | q.s. |
| Water | ad 100 | ad 100 | ad 100 | ad 100 | ad 100 | ad 100 |
| Neutralization medium | qs | qs | qs | qs | qs | qs |
| (sodium hydroxide, | | | | | | |
| pottasium hydroxide) | | | | | | |

The above formulation may of course contain one or more actives among the following list: Zinc Pyrithione, Piroctone Olamine, Ketoconazole, Tropolone, Hinokitol, Selenium Sulfide, Salicylic Acid, Climbazole, Sodium Salicylate, Ciclopiroxolamine, Neem, Basilic oil, Ichtammol, Melaleuca Alternifolia, Centaurea Cyanus, Melia Azadirachta, Farnesol, Sulfur, Clotrimazole, Crotamiton, Zinc Salicylate, Tussilago farfara, Arctium lappa, Zinc Sulfate, Rosmarinus officinalis, Myrtrimonium Bromid, Lactic Acid, Chlorohexidine Digluconate, Phenoxyisopropanol, Isopropanol, Farnesol, Glycolic Acid, Tannic acid, Alcohol, Triclosan, Zinc Gluconate, Zinc PCA, Camphor, Aluminium salts, Sodium Lactate, Polyaminopropyl Biguanide, Zinc Acetate, Triethyl Citrate, Ethylhexylglycerol, Aluminium Circonium, Tetrachlorohydrex GLY, Pentetic Acid, Diisopropylamine Aminoethylpropanol, Zinc Ricinoleate, Aluminium Sesquichlorohydrate, Lactic Acid, Triclosan.

### I) Aqueous and Hydroalcoholic Formulations [Amounts are Given in % by Weight of the Formulation]

| | A | E | C | D | E | F |
|---|---|---|---|---|---|---|
| Ethanol | 50 | 5 | 2 | 40 | 15 | |
| Hydroxyethylcellulose | 0.5 | | | | | |
| Acrylates/C10-30 Alkyl | | | | 0.3 | 0.6 | |
| Acrylate Crosspolymer | | | | | | |
| Cocoatnidopropylbetaine | | | 0.3 | | | |
| UVASorb ® K2A | | | | | 2 | |
| Uvinul ® APlus | 5 | | | | | |
| Butyl | 0.5 | | | 3 | | |
| Methoxydibenzoylmethane | | | | | | |
| Disodium Phenyl | | 2 | 1 | | | |
| Dibenzimidazol | | | | | | |
| Tetrasulfonate | | | | | | |
| Phenylbenzimidazol Sulfonic | | 5 | 3 | | 2 | 4 |
| acid | | | | | | |
| Ethylhexyl | 10 | | | | 3 | |
| Methoxycinnamate | | | | | | |
| Diethylhexyl Butamido | | | | 3 | | |
| Triazon | | | | | | |
| Ethylhexyl Triazon | | | | | 2 | |
| Octocrylene | | | | 5 | | |
| Composition of example 1 | 2.5 | 0.75 | 1.5 | 3.0 | 3.5 | 4.0 |
| C₁₂₋₁₅ Alkyl Benzoate | | | | 3 | | |
| C18-36 Triglycerid Fatty acid | | | | 1 | | |
| Butylene glycol | 2 | | | | | |
| Dicaprylate/Dicaprate | | | | | | |
| C12-13 Alkyl Tartrate | | | | | 5 | |
| Cyclomethicone | 4 | | | 2 | | |
| Insect Repellent ® 3535 | | | | 5 | | |
| Dimethicone | | | | | 3 | |
| PVP Hexadecen Copolymer | | 0.5 | | 1 | | 0.5 |
| Ethylhexyloxyglycerol | | 0.5 | | | | |
| Glycerol | 5 | 7 | 3 | 8 | | S |
| Butylene glycol | | | 5 | | 5 | |
| Metylpropandiol | | | | 4 | | |
| Vitamin E Acetate | | 0.3 | 0.2 | 0.5 | | |
| Panthenol | 0.5 | | 0.2 | | | 0.3 |
| Creatinine | | | 0.01 | | 0.02 | |
| Creatin | | | 0.1 | | 0.2 | |
| PEG-40 Hydrated Ricinus oil | | 0.5 | 0.3 | | | 0.5 |
| Trisodium EDTA | 0.3 | 0.2 | 0.2 | 0.2 | 0.2 | 0.5 |
| Preservative | q.s. | q.s. | q.s. | q.s. | q.s. | q.s. |
| Sodium hydroxide | q.s. | q.s. | q.s. | q.s. | q.s. | q.s. |
| Parfum, Dye | q.s. | q.s. | q.s. | q.s. | q.s. | q.s. |
| Water | ad 100 | ad 100 | ad 100 | ad 100 | ad 100 | ad 100 |

The above formulation may of course contain one or more actives among the following list: Zinc Pyrithione, Piroctone Olamine, Ketoconazole, Tropolone, Hinokitol, Selenium Sulfide, Salicylic Acid, Climbazole, Sodium Salicylate, Ciclopiroxolamine, Neem, Basilic oil, Ichtammol, Melaleuca Alternifolia, Centaurea Cyanus, Melia Azadirachta, Farnesol, Sulfur, Clotrimazole, Crotamiton, Zinc Salicylate, Tussilago farfara, Arctium lappa, Zinc Sulfate, Rosmarinus officinalis, Myrtrimonium Bromid, Lactic Acid, Chlorohexidine Digluconate, Phenoxyisopropanol, Isopropanol, Farnesol, Glycolic Acid, Tannic acid, Alcohol, Triclosan, Zinc Gluconate, Zinc PCA, Camphor, Aluminium salts, Sodium Lactate, Polyaminopropyl Biguanide, Zinc Acetate, Triethyl Citrate, Ethylhexylglycerol, Aluminium Circonium, Tetrachlorohydrex GLY, Pentetic Acid, Diisopropylamine Aminoethylpropanol, Zinc Ricinoleate, Aluminium Sesquichlorohydrate, Lactic Acid, Triclosan.

J) Cosmetic Foam [Amounts are Given in % by Weight of the Formulation]

| Emulsion | A | B | C |
|---|---|---|---|
| Stearic acid | 2 | 2 | |
| Palmitic acid | | | 1.5 |
| Cetyl alcohol | 2.5 | 2 | |
| Stearyl alcohol | | | 3 |
| PEG-100 Stearate | | | 3.5 |
| PEG-40 Stearate | | 2 | |
| PEG-20 Stearate | 3 | | |
| Sorbitanstearate | | 0.8 | |
| C₁₂₋₁₅ Alkyl Benzoate | 5 | | |
| C₁₂₋₁₃ Alkyl Tartrate | | | 7 |
| Butylene glycol | | 6 | |
| Dicaprylate/Dicaprate | | | |
| Dicaprylyl Ether | | | 2 |
| Cyclomethicone | | 2 | 3 |
| Butylene glycol | 1 | | |
| Isohexadecane | 2 | | |
| Methylpropandiol | | | |
| Propylene glycol | | | 5 |
| Glycerol | 5 | 7 | |
| UVASorb ® K2A | | | 2 |
| Uvinul ® A Plus | 2 | 3 | |
| Composition of example 1 | 0.5 | 1.0 | 1.5 |
| Parsol SLX ® | | 3 | |
| Homosalate | | 5 | |
| Phenylbenzimidazol Sulfonic | | 2 | 2 |
| acid | | | |
| Benzophenone-3 | 2 | | |
| Octylsalicylate | | 5 | |
| Octocrylene | 2 | | |
| Bis-Ethylhexyloxyphenol | | 3 | |
| Methoxyphenyltriazin | | | |
| 2,2′-Methylen-bis-(6-(2H- | | | 8 |
| benzotriazol-2-yl)-4-(1,1,3,3- | | | |
| tetramethylbutyl)-phenol) | | | |
| 2,4,6-Tris-(biphenyl)-1,3 5- | 5 | | 4 |
| triazin | | | |
| C8-C16 Alkylpolyglycoside | 1 | | |
| Vitamin E Acetate | 0.6 | 0.5 | 0.2 |
| Creatin/Creatinine | | | 0.5 |
| BHT | | | 0.1 |
| Na₂H₂EDTA | 0.50 | | |
| Parfum, preservative | q.s. | q.s. | q.s. |
| Dye | q.s. | q.s. | q.s. |
| Sodium hydroxide | q.s. | | q.s. |
| Potassium ydroxide | | q.s. | |
| Water | ad 100 | ad 100 | ad 100 |

Continuation cosmetic foam [amounts are given in % by weight of the formulation]

| Emulsion | D | E | F | G |
|---|---|---|---|---|
| Stearic acid | 2 | | | |
| Palmitic acid | | | 3 | 3 |
| Cetyl alcohol | 2 | 2 | | |
| Cetylstearylalcohol | | | 2 | 2 |
| Stearyl alcohol | | | | |
| PEG-100 Stearate | | 4 | | |
| PEG-40 Stearate | 2 | | | |
| PEG-20 Stearate | | | 3 | 3 |
| Sorbitanstearate | 0.8 | | | |
| Tridecyl Trimellitate | | 5 | | |
| C₁₂₋₁₅ Alkyl Benzoate | | | 3 | 3 |
| Butylene glycol | 8 | | | |
| Dicaprylate/Dicaprate | | | | |
| Octyldodecanol | | 2 | | |
| Cocoglyceride | | | | 2 |
| Dicaprylyl Ether | | | 2 | 2 |
| Cyclomethicone | | | | |
| Dimethicone | 1 | | 2 | 2 |
| Isohexadecane | | 3 | | |
| Methylpropandiol | | 4 | | |
| Propylene glycol | | | | |
| Glycerol | 5 | | 6 | 6 |
| NeoHeliopan ® AP | | 2 | | |
| Phenylbenzimidazol | 1 | | | 1 |
| Sulfonic acic | | | | |
| Composition of example 1 | 0.75 | 1.5 | 3 | 6 |
| Ethylhexylmethoxycinnamate | 5 | | 4 | 4 |
| Ethylhexyltriazon | | 2 | | 1 |
| Eusolex ® T-AVO | 2 | | | |
| Diethylhexylbutamidotriazon | 1 | | | |
| Butyl Methoxydibenzoylmethane | 2.5 | | 2 | 2 |
| Bis-Ethylhexyloxyphenol | 2 | | | |
| Methoxy-phenyltriazin | | | | |
| Vitamin E Acetate | 0.2 | | 0.3 | 0.3 |
| Na₂H₂EDTA | | | | |
| Parfum, preservative | | | | |
| Dye | | | | |
| sodium hydroxide | | q-s. | q.s. | |
| Triethanolamine | q.s. | | | q.s. |
| Water | ad 100 | ad 100 | ad 100 | ad 100 |

The above formulation may of course contain one or more actives among the following list: Zinc Pyrithione, Piroctone Olamine, Ketoconazole, Tropolone, Hinokitol, Selenium Sulfide, Salicylic Acid, Climbazole, Sodium Salicylate, Ciclopiroxolamine, Neem, Basilic oil, Ichtammol, Melaleuca Alternifolia, Centaurea Cyanus, Melia Azadirachta, Farnesol, Sulfur, Clotrimazole, Crotamiton, Zinc Salicylate, Tussilago farfara, Arctium lappa, Zinc Sulfate, Rosmarinus officinalis, Myrtrimonium Bromid, Lactic Acid, Chlorohexidine Digluconate, Phenoxyisopropanol, Isopropanol, Farnesol, Glycolic Acid, Tannic acid, Alcohol, Triclosan, Zinc Gluconate, Zinc PCA, Camphor, Aluminium salts, Sodium Lactate, Polyaminopropyl Biguanide, Zinc Acetate, Triethyl Citrate, Ethylhexylglycerol, Aluminium Circonium, Tetrachlorohydrex GLY, Pentetic Acid, Diisopropylamine Aminoethylpropanol, Zinc Ricinoleate, Aluminium Sesquichlorohydrate, Lactic Acid, Triclosan.

Testdesign “Antiinflammatory Efficacy”:

Comparison of two emulsions: emulsion A contains 1% ZnO, emulsion B contains 1% compositions according to the invention. In case that emulsion B attenuates erythema to at least the same extent as emulsion A, but preferably better, we conclude that additional emulsion B in the emulsion A may boost the anti-inflammatory activity of ZnO.

Remarks: ZnO is well known as being anti-inflammatory. The erythema is artificially induced by UV light at the inner human forearm. Erythema is redness of the skin caused by capillary congestion. It can be caused by infection, massage, electrical treatments, acne medication, allergies, exercise or solar radiation (sunburn), and waxing and plucking of the hairs any of which can cause the capillaries to dilate, resulting in redness.

Test Design “Reduction of Sebum Excretion”

1. Using a Sebumeter. The lipid (oil) probe or Sebumeter, is a device that measures the amount of oil in the skin by shining a diode light source through a membrane that is soaked with the skin lipids when it is placed on the skin surface. The more lipid on the membrane, the less light passes through to a photo sensor.

The Sebumeter is a highly accurate method of measuring the oil content of the skin.

Method: Simply press the lipid probe cassette to the skin surface in the area you wish to test for an automatically timed period, then insert the cassette back in to the analyser for reading of the lipid level of the skin.

Test Design “Anti Acne (Bacterial Test)”

Identical to test design antimicrobial efficacy but testing different microbes (Propionibacterium acnes, Staphylococcus epidermidis, Staphylococcus aureus—results in FIG. 1 and FIG. 6 for the microorganism Propionibacterium acnes).

Test Design “Anti Viral”

The method described in the publication of Fumio Shimizu, Yoshinobu Shimizu, Katsuo Kumagai, Antimicrobial Agents and Chemotherapy, 1976, 10(1), 57-63 could be used for that purpose.

Test Design “Anti Dental Plaque”

1. identical to test design antimicrobial efficacy but testing different microbes: Actinomyces viscosus, Bacteroides intermedia, Fusobacterium nucleatum, Porphyromonas gingivalis, Streptococcus mutans, Streptococcus salivarius, Streptococcus sanguinis

2. Evaluation of the amount of dental plaque

Estimating the amount of plaque

The Quigley Hein plaque index:

This index evaluates the plaque revealed on the check (buccal) side and the tongue (lingual) side of the teeth on a scale from 0-5 where:

0=no plaque

1=isolated flecks of plaque near the gingival (gum) margin

2=a 1 mm band of plaque at the gingival margin

3=up to ⅓ of the surface covered with plaque

4=disclosed plaque from ⅓ to ⅔ of the surface

5=disclosed plaque on more than ⅔ of the surface

References: Quigley, G A and Hein, J W, Comparative cleaning efficacy of manual and power brushing, J. Am. Dent. Assoc. 1962, 65,26-29 Addy, M, M. A. Slayne, and W. G. Wade, 1993, Methods for the Study of Dental Plaque formation and control, IN Denyer, S. P., S. P. Gormann and M. Sussmann, Microbial Biofilms: Formation and Control, Blackwell Scientific Publications, Oxford

Test Design “Wound Healing”

Wound Site Measurement Techniques

The purpose of any wound measurement is to monitor the progress of healing through changes in the length, width, area or volume of a wound. This can be done using the following techniques:

Simple measurement: The simplest and cheapest method is to calculate the wound surface area by measuring its linear dimensions with a tape measure or ruler. However, this two-dimensional method assumes that the wound has a geometric surface shape, for example a rectangle (length×width), a circle (diameter×diameter) or an oval (maximum diameter×maximum diameter perpendicular to the first measurement). An alternative method of calculating wound surface area is based on the formula for an ellipse (length×width×0.785).

Wound tracing: Another two-dimensional wound measurement tool is wound tracing, in which a pen is used to trace the outline of the wound directly onto sterile transparent film. Wound tracing can be performed at the bedside using a minimum of equipment and requires no special skills or training on the part of the clinician. Each tracing in a sequence is easy to compare with the others and tracing is relatively unobtrusive for the patient. Tracings can be immediately stored in the patient's records and could be entered into a data processing system using a simple scanner. The most significant limitation of wound tracing is deciding where the boundary of the wound lies, which affects the reliability and accuracy of the technique. A Kundin gauge: This is a commercially available three-dimensional ruler used to calculate wound area and volume.

Moulds: A three-dimensional mould of the wound can be created by taking a cast of the wound cavity using a saline or alginate filling.

Scaled photographs: This two-dimensional method of assessment uses a photograph that has been processed by a special scanner so that a scaled ruler is incorporated at the edge of the photograph. The ruler is used to calculate length and width, which are expressed in simple measurements. Scaled photographs are useful for comparison but there is the potential for magnification errors.

Planimetrics: This method measures volume by creating a two-dimensional or planar image from a photograph or wound tracing. A transparent sheet of graph paper is laid over the photograph or wound tracing, either manually or using a computer, and the number of complete graph squares within the boundaries of the wound are added up to produce a scale area calculation.This can then be stored in the patient's records or be entered into a data processing system.

Computerised stereophotogrammetry: Originally developed for land surveying, computerised stereophotogrammetry uses two pictures of the same area taken from different known positions to produce a three-dimensional image. A computerised matching algorithm searches for corresponding points in the two images and then computes the height of each point, based on the distance between corresponding points in the pair of pictures.

Some of the above techniques can be used in conjunction with others. For example, Yenidunya and Demirseren used a technique in which the contour of a wound was traced on a transparent sheet overlaid on a digital photograph of the wound displayed on a computer screen. The resulting trace, which was relatively unobtrusive for the patient and could be stored digitally, was used for both preoperative planning and wound assessment.

Non-invasive assessment methods such as measurement of the transepidermal water loss (TEWL) allow a continuous follow-up of cutaneous processes with impairment of the epidermal barrier function. LEVY J. J.; VON ROSEN J.; GASSMÜLLER J.; KUHLMANN R. K. ; LANGE L. ; Dermatology, 1995, vol. 190, n^{o} 2, pp. 136-141

Other methods can be used like for instance Laser Doppler, scanning, Laser Doppler Flowmetry, Transcutaneous Oxygen measurement.

Testdesign “Hair Loss”:

Trichogramm (Semi-Invasive Process)

The trichrogramm is a methode, which is often used in order to measure hair loss, or to quantify the effect of pharmaceuticals designed for hair loss.Thanks to microscopic differenciation and the different phases of hair growth on epilated hair, it is possible to use the trichrogramm in order to
Objectivate and standardize hair loss
Measure the activity of hair loss
Therapy control

With classical trichrogramms, it is necessary to do painful epilations. With androgen alopecia the epilation should occur in a thinning out head area (back of the head). The microscopic counting of the skin roots allows the calculation of the ratio Angen-/Telogenrate, which also means a conclusion of the intensity of the hair loss. Hair is first parted and a hair row is then prepared. In order to have a trichrogramm, which can be exploited, it is necessary to:

To have more than 50 hairs epilated. Hair is first parted and a row is prepatated. Hair is epilated in the direction of hair growth. The epilated hair is then put on a glass slide (fixed with scotch tape) and then cutted off. The hair can either be moisturized with a drop of water and then microscopically analyzed. It is also possible to use a fixation liquid for microscopy (Eukitt). The three hair growth phases are observed on the trichrogramm according to their corresponding duration in the phases.

1. Composition comprising zinc oxide, barium sulphate and bound silver ions.

2. Composition according to claim 1, characterized in that the silver ions are ionically bound to zinc oxide and/or barium sulphate.

3. Composition according to claim 1, characterized in that the ratio of zinc oxide to barium sulphate to bound silver ions is in the range of 12000:2000:1 to 8:1:1, based on the weight.

4. Composition according to claim 1, characterized in that the ratio of zinc oxide to barium sulphate to bound silver ions is 2500:500:1, based on the weight.

5. Composition according to claim 1, characterized in that zinc oxide, barium sulphate and bound silver ions have a synergistic antimicrobial activity, synergistic anti irritation property, synergistic anti inflammation property, synergistic wounds healing properties, synergistic anti acne properties, synergistic anti hair loss properties, synergistic properties on the reduction of sebum excretion, synergistic UV protection properties and/or synergistic effects against skin diseases.

6. Composition according to claim 1, further comprising an additional compound which is platelet-shaped, spherical or needle-shaped.

7. Composition according to claim 1, further comprising an additional compound which is selected from the group of natural or synthetic mica, SiO₂, TiO₂, BiOCl, Aluminium oxide, glass, micaceous iron oxide, graphite, oxidised graphite, aluminium oxide coated graphite, basic lead carbonate, barium sulphate, chromium oxide, BN, MgO, magnesium fluoride, Si₃N₄, and/or metals.

8. Composition according to claim 1, further comprising an additional compound which is coated with one or more layers or deposits of BiOCl and/or transparent, semitransparent or opaque, selectively or nonselectively absorbing or nonabsorbing metal oxides, metal suboxides, metal oxide hydrates, metals, metal sulphates, metal nitrides, metal oxynitrides, metal fluorides and/or mixtures of these materials.

9. Composition according to claim 8, characterized in that the one or more layers of BiOCl and/or transparent, semitransparent or opaque, selectively or nonselectively absorbing or nonabsorbing metal oxides, metal suboxides, metal oxide hydrates, metals, metal sulphates, metal nitrides, metal oxynitrides, metal fluorides and/or mixtures of these materials are arranged as alternating layers of transparent, semitransparent or opaque, selectively or nonselectively absorbing or nonabsorbing metal oxides, metal suboxides, metal oxide hydrates, metals, metal sulphates, metal nitrides, metal oxynitrides, metal fluorides and/or mixtures of these materials or BiOCl with a refractive index n>1.8 and transparent, semitransparent or opaque, selectively or nonselectively absorbing or nonabsorbing metal oxides, metal suboxides, metal oxide hydrates, metals, metal sulphates, metal nitrides, metal oxynitrides, metal fluorides and/or mixtures of these materials with a refractive index n<1.8.

10. Composition according to claim 8, characterized in that the transparent, semitransparent or opaque, selectively or nonselectively absorbing or nonabsorbing metal oxides, metal suboxides, metal oxide hydrates, metals, metal sulphates, metal nitrides, metal oxynitrides, metal fluorides and/or mixtures of these materials additionally contain organic and/or inorganic colorants or elements as dopant.

11. Composition according to claim 1, characterized in that zinc oxide and barium sulphate are deposited on the surface of the additional compound.

12. Composition according to claim 11, characterized in that the additional compound is coated with particles of barium sulphate having an average diameter of 0.1 to 2.0 μm and with needle crystal particles of zinc oxide having an average major-axis diameter of 0.05 to 1.5 μm, wherein the amount in parts by weight of said barium sulphate is smaller than that of said zinc oxide, relative to the amount of said additional compound.

13. Composition according to claim 1, characterized in that the composition comprises, based on the composition, 30 to 50 weight percent an additional compound, 40 to 60 weight percent zinc oxide, 5 to 10 weight percent barium sulphate and 0.005 to 5 weight percent bound silver ions calculated as silver oxide.

14. Composition according to claim 1, characterized in that the composition comprises, based on the composition, 40 weight percent an additional compound, 50 weight percent zinc oxide, 9.98 weight percent barium sulphate and 0.02 weight percent bound silver ions calculated as silver oxide.

15. Composition according to claim 1, characterized in that it comprises an additional compound which is mica.

16. Method for the preparation of Compositions according to claim 1 comprising the agitation of a suspension comprising zinc oxide, barium sulphate and a silver salt.

17. Method according to claim 16, characterized in that the silver salt is silver oxide or silver acetate.

18. Method according to claim 16, characterized in that zinc oxide and barium sulphate are deposited on the surface of an additional compound.

19. Method according to claim 18, characterized in that the additional compound is coated with particles of barium sulphate having an average diameter of 0.1 to 2.0 μm and with needle crystal particles of zinc oxide having an average major-axis diameter of 0.05 to 1.5μ wherein the amount in parts by weight of said barium sulphate is smaller than that of said zinc oxide, relative to the amount of said additional compound.

20. Method according to claim 18, characterized in that the additional compound is mica.

21. Method according to claim 16, characterized in that the preparation is performed in water, ethanol, methanol, 1-propanol, 2-propanol and/or mixtures thereof.

22. Method according to claim 16, characterized in that the preparation temperature is between 10 and 60° C.

23. Method according to claim 16, characterized in that the amount of the silver salt is in the range of 0.005% to 5% by weight, based on the composition.

24. A method for the inhibition of the growth and/or progeny of microorganisms, for inhibition of irritation, of inflammation, of acne formation, of sebum excretion, of hair loss, for UV protection and/or for wound healing, comprising administering a composition according to claim 1 to a subject in need thereof.

25. A formulation and/or application selected from the group of cosmetic formulations, paints, inks, food colouring, home care products, animal care products, products for personal and work hygiene, contact lenses, chromatography materials, medical equipment, protective topicals, pharmaceutical, especially dermatological formulations, lacquers, coatings and/or plastics, comprising a composition according to claim

26. Formulation according to claim 25, characterized in that the formulation is in the form of solutions, suspensions, emulsions, pasta, ointments, gels, creams, lotions, powders, oils, pencils, deodorants-cremes, gels, lotions, emulsions, deodorant sticks, Roll-ons, sprays, pump sprays or lacquers.

27. Formulation according to claim 25, characterized in that they comprise at least one compound selected from the group consisting of suitable substrates for microorganisms, characterized in that the suitable substrate for microorganisms is preferably selected from the group consisting of alkanes, alkenes, alkines, with or without functional groups, sugars, polyols, alcohols, saturated or unsaturated carboxylic acids, proteins, amino acids, water, fatty acids, waxes, fats, mineral oils, salts, hormones, steroids, vitamins and/or derivatives or salts thereof, or characterized in that they additionally comprise preservatives and antimicrobial agents, or characterized in that they additionally comprise antibiotics, characterized in that the antibiotics are preferably selected from the group of Beta-lactam, Vancomycin, Macrolides, Tetracyclines, Quinolones, Fluoroquinolones, Nitrated compounds, Aminoglycosides, Phenicols, Lincosamids, Synergistins, Fosfomycin, Fusidic acid, oxazolidinones, Rifamycins, Polymixynes, Gramicidins, Tyrocydine, Glycopeptides, Sulfonamides or Trimethoprims, or characterized in that they additionally comprise one or more UV filters, or characterized in that they additionally comprise at least one self-tanning agent, or characterized in that they additionally comprise dyes and coloured pigments, or characterized in that they additionally comprise at least one antioxidant, or characterized in that they additionally comprise vitamins, or characterized in that they additionally comprise skin-protecting or skin-care active ingredients, or characterized in that they additionally comprise at least one photostabiliser.

28-44. (canceled)
